(19) United States
(12) Patent Application Publication
(10) Pub. No.: US 2023/0245725 A1
(43) Pub. Date: Aug. 3, 2023

(54) Means and Methods for the Prediction of Amyloid Core Sequences

(71) Applicants: VIB VZW, Gent (BE); KATHOLIEKE UNIVERSITEIT LEUVEN, K.U.LEUVEN R&D, Leuven (BE)

(72) Inventors: Frederic Rousseau, Sint-Martens-Bodegem (BE); Joost Schymkowitz, Meensel-Kiezegem (BE); Gabriele Orlando, Leuven (BE); Nikolaos Louros, Kessel-Lo (BE)

(21) Appl. No.: 17/927,527

(22) PCT Filed: May 21, 2021
(86) PCT No.: PCT/EP2021/063691

§ 371 Date: Nov. 23, 2022

(30) Foreign Application Priority Data
May 26, 2020 (EP) 20176563.3

(51) Int. Cl. G16B 40/20; G16B 15/20

(52) U.S. Cl. CPC G16B 40/20; G16B 15/20

(57) ABSTRACT

The present methods and systems generally relate to the biomedical field and relate to subfields of computational biology and bioinformatics. More, specifically the invention provides an artificial intelligence algorithm which can identify aggregation prone regions, particularly amyloid sequences in a protein.

### CROSS-REFERENCE TO RELATED APPLICATIONS

This application is a national phase entry under 35 U.S.C. § 371 of International Patent Application PCT/EP2021/063691, filed May 21, 2021, designating the United States of America and published in English as International Patent Publication WO 2021/239629 on Dec. 2, 2021, which claims the benefit under Article 8 of the Patent Cooperation Treaty to European Patent Application Serial No. 20176563.3, filed May 26, 2020, the entireties of which are hereby incorporated by reference.

### FIELD OF THE INVENTION

The present methods and systems generally relate to the biomedical field and relate to subfields of computational biology and bioinformatics. More, specifically the invention provides an artificial intelligence algorithm which can identify aggregation prone regions, particularly amyloid sequences in a protein.

### Introduction to the Invention

The amyloid cross-beta state is a polypeptide conformation that is adopted by 36 proteins or peptides associated to human protein deposition pathologies¹. It also constitutes the structural core of a growing number of functional amyloids in both bacteria and eukaryotes^{2,3}. Beyond these bona fide functional and pathological amyloids it has been demonstrated that many if not most proteins can adopt an amyloid-like conformation upon unfolding/misfolding⁴. This has led to the notion that just like the alfa-helix or beta-sheet, the amyloid state is a generic polypeptide backbone conformation but also that amino acids have different propensities to adopt the amyloid conformation⁵. Initially, it was observed that amyloid-like aggregation correlates with hydrophobicity, beta-strand propensity, and (lack of) net charge⁶. This triggered the development of aggregation prediction algorithms that essentially evaluate the above biophysical propensities^{7,8}. Others extended to scaling residue propensities between protein folding and aggregation^{9,10}. These algorithms confirmed the ubiquity of amyloid-like propensity in natural protein sequences and particularly in globular proteins as it was estimated that 15-20% of residues in a typical globular domain are within aggregation-prone regions (APRs)^{11,12}. These APRs are sequence segments of six to seven amino acids in length on average and are mostly buried within the protein structure where they constitute the hydrophobic core stabilizing tertiary protein structure^{13,15}. On the other hand, the increasing identification of both yeast prions and functional amyloids clearly indicated that amyloid sequence space is not monolithic and that more polar/less aliphatic sequences represent important alternative populations of amyloid sequence space³. The limited sensitivity of the above cited algorithms to specifically identify these other subpopulations confirmed the underestimated sequence versatility of the amyloid conformation. Indeed, more recently the role of amyloid-like sequences in proteins mediating liquid-liquid phase transitions again demonstrates the ubiquity of the amyloid in biological function and further withers the image of the amyloid state as a predominantly disease and/or toxicity associated protein conformation¹⁶⁻¹⁸. Rather, this suggests that like globular protein folding, amyloid assembly is a matter of kinetic and thermodynamic control that can be evolutionary tuned by sequence variation and selection. Efforts to develop aggregation predictors that can identify a broader spectrum of amyloid sequences have increased over the years¹⁹. Such approaches focused on identifying position-specific patterns by reference to accumulated experimental data of APRs′, or by using energy functions of cross-beta pairings²³. Recently developed meta-predictors produce consensus outputs by combining previous methods, in an attempt to boost performance^{24,25}. Indirect structured-based methods were initially developed by considering secondary structure propensities^{26,27}. Complementary studies extended this notion by suggesting that disease-related amyloids form β-strand-loop-β-strand motifs²⁸. There remains however still a need to develop reliable algorithms to detect amyloid sequences beyond their current know boundaries.

### SUMMARY OF THE INVENTION

In the present invention, we have used a machine learning approach to identify amyloid sequences in proteins. Specifically, the invention provides an algorithm, which is herein further designed as Cordax, which is an exhaustively trained regression model that leverages a substantial library of curated template structures combined with machine learning. Cordax not only detects APRs in proteins, but also predicts the structural topology, orientation and overall architecture of the resulting putative fibril core. To validate the accuracy of our predictions, we designed a screen of 96 newly predicted APRs and experimentally determined their aggregation properties. Using this approach, we identified less hydrophobic polar and charged aggregation prone sequences that increasingly uncouple solubility and amyloid propensity, closely resembling characteristics of phase-separation inducers. Clustering by t-Distributed Stochastic Neighbour Embedding reveals the heterogeneous substructure of amyloid sequence space consisting in varying clusters corresponding to sequences compatible with globular structure, functional scaffolding amyloids, N/Q/Y rich prions, helical peptides and intrinsically disordered sequences. Together, the structural exploration performed here demonstrates that the field now gathered sufficient structural and sequence information to start classifying amyloids according to different structural and functional niches. Just like for globular proteins in the 1980s, this will allow to fine-tune both general and context-dependent structural rule learning allowing to manipulate and design amyloid structure and function.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A-1F: Development of the regression model pipeline. (FIG. 1A) Processing steps of the peptide fragment library. Crystal contact information was used to generate fibril cores from isolated PDB structures. Structures containing multiple packing interfaces were split into individual templates (1), which were in turn split into hexapeptide core fragments (2). (FIGS. 1B and 1C) Correlation plot of interface energies calculated using FoldX. Top half shows correlation values with scatter plots indicated at the bottom half. Rejected fragments sharing low shape complementarity (shown in yellow) have correlating weak van der Waals interfaces, as well as poor solvation energies for hydrophobic side chains compared to the remaining library (indicated in purple). (FIGS. 1D and 1E) Promiscuity sorting of the structural library performed as a two-step cross-threading process. Circular histograms highlight 3 major promiscuous structures (n>5) which were removed during the primary (PDB ID: 1YJO, 3FR1 and 6CFH_3) and secondary step (PDB ID: 3FOD, 4XFN and 4W67_2). (FIG. 1F) Schematic representation of Cordax training and the derived pipeline.

FIGS. 2A-2D: Benchmarking of CORDAX. (FIG. 2A) ROC curve analysis for Cordax and six other state-of-art methods against WALTZ-DB 2.0. For WALTZ, TANGO and MetAmyl, FPR stops at earlier rates due to minimal scoring variations. (FIGS. 2B and 2C) Cordax score distribution compared to other tools. The regression model achieves better scoring separation for predictions between amyloid-forming (shown in blue) and non-amyloid sequences (shown in red). Density plots for WALTZ, TANGO, MetAmyl and GAP are scaled due to the overrepresentation of unscored values or false positives, respectively. (FIG. 2D) Performance metrics comparison indicating Cordax superiority to other sequence predictors (MCC=0.57, F1=0.73 and AUC=0.87).

FIGS. 3A-3F: Amyloid-forming properties of the peptide screen designed by employing Cordax. (FIGS. 3A and 3B) Measured pFTAA and ((FIGS. 3C and 3D) Th-T fluorescence of synthetic peptides following rotation at 200 μM for 5 days. Data are presented as mean values with standard deviation (SD) of independent replicates (n=6). Significant differences were computed using unpaired t-test by comparing to vehicle controls, shown in black bars (Denoted level of significance: n.s., not significant, *p-value<0.05, **p-value<0.01, ***p-value<0.001, ****p-value<0.0001). ((FIG. 3E) Electron micrographs of amyloid fibrils formed by Th-T or pFTAA binding peptides. ((FIG. 3F) Suspensions of amyloid fibrils bind Congo red as displayed under bright field illumination (BF) and exhibit typical for amyloids apple-green birefringence under crossed-polarised light (CP). Scale bars: 500 urn.

FIGS. 4A-4M: Cordax identifies surface-exposed aggregation nucleators spanning residues that are typically considered unconventional for amyloid fibril formation. (FIG. 4A) Schematic representation of Cordax-predicted topological models for APRs charted against the cognate native crystal structure of the amyloidogenic protein Ure2p. (FIGS. 4B-4H) Surface representation of folded structures for (FIG. 4B) Ure2p, (FIG. 4C) RepA, (FIG. 4D) Acylphosphatase-2, (FIG. 4E) Sup35, (FIG. 4F) Prolactin, (FIG. 4G) Lactoferrin and (FIG. 4H) Kerato-epithelin reveals that aggregation nucleators uniquely identified by Cordax (highlighted in red) are primarily exposed to the surface of proteins, compared to segments of joint prediction (shown in blue) which are predominantly buried within the hydrophobic core of the native fold. Cordax-specific predicted APRs produced lower volumetric burial values, calculated using FoldX, for (FIG. 4I) side chain and (FIG. 4J) main chain groups indicating that they are considerably exposed compared to jointly identified nucleators. (FIG. 4K) Partition coefficients indicate that Cordax-specific APRs are significantly more soluble compared to typically predicted sequences that are primarily hydrophobic and therefore insoluble. Solubility regions (vi, very insoluble; i, insoluble; n, neutral; s, soluble; vs, very soluble) are shown as coloured backgrounds⁷². Significant differences were computed using unpaired t-test statistical analysis (****p-value<0.0001). (FIG. 4I) Surface-exposed Cordax-specific APRs are composed of residues with a 20% increase in polar and charged side chains, in expense of hydrophobic residues. (FIG. 4M) Secondary structure analysis, using FoldX, indicates that Cordax identifies several APRs that reside in α-helical or unstructured regions within the native fold, suggesting that amyloidogenic proteins may harbour a plethora of exposed conformation switches that can act as potential nucleators of amyloid fibril formation, under suitable misfolding conditions.

FIGS. 5A-5I: t-SNE 2D-representation of the known experimentally determined amyloidogenic sequence space. (FIG. 5A) State-of-the-art sequence-based methods predict amyloid sequences, with (shown in cyan) or without Cordax (shown in yellow), that are grouped together in a major landing cluster and two islands. Cordax predictions (shown in purple) transgress towards areas of amyloid-forming sequences that remain undetected by most methods (shown in black). (FIG. 5B) Clustering of the t-SNE map using basic physicochemical properties and amino acid composition of the amyloid peptides. Each data point is colour-coded based on the sorting scheme shown in the legend and background areas are used to pinpoint the major clusters of each defined category. The clustering scheme was defined by characterising the t-SNE map using peptide (FIG. 5C) hydrophobicity, (FIG. 5D) net charge, (FIG. 5E) aliphatic index, (FIG. 5F) secondary structure propensity and percentage content of (FIG. 5G) aromatic or (FIG. 5H) short residue side chains. (FIG. 5I) Highly soluble, yet amyloid-forming, sequences are the largest portion of new amyloid sequences identified by Cordax. Partition coefficient analysis reveals that APRs identified by Cordax are primarily soluble sequences compared to easy to identify sequences of joint prediction. On the other hand, APRs that remain hard to detect are characterised by higher solubilities. Solubility regions (vi, very insoluble; i, insoluble; n, neutral; s, soluble; vs, very soluble) are shown as coloured backgrounds. Significant differences were computed using unpaired t-testing (Denoted level of significance: n.s., not significant, **p-value<0.01, ****p-value<0.0001).

FIGS. 6A-6F: High-precision recognition of amyloid fibril structural architectures using Cordax. (FIG. 6A) Prediction accuracy comparison of Cordax to the only publicly available structural predictors, Fibpredictor and 3D-profile. For comparison, methods were run against a non-redundant sequence set extracted from amyloid-forming peptide interfaces. (FIG. 6B) Model topologies, predicted by applying Cordax (shown in orange), strongly superimpose to matching solved structural layouts of amyloidogenic nucleators (shown in magenta), as indicated by the reported minor RMSD values. (FIG. 6C) Sequence identity contribution for template selection during cross-threading analysis of the Cordax structural library. Alignment scores for selected models matching the template sequences (shown in Table 1) compared to mismatching template selections of similar or different topological layouts (shown in Table 2). (FIG. 6D) Alignment scores of the APRs newly identified by Cordax to the sequence of the selected templates, plotted against their corresponding model ranks. (FIG. 6E) Structural alignment of Cordax outputs to experimentally determined 3D-structures. Models were calculated for three aggregation prone sequences derived from CsgA curli forming protein (PDB IDs: 6G8C, 6G8D and 6G8E, respectively) and a peptide mutant sequence derived from Aβ amyloid peptide (PDB ID: 5TXH). Predicted topologies are overlapping representations of the experimentally determined amyloid fibril cores, (FIG. 6F) as displayed by a direct comparison to other software.

FIGS. 7A-7I: Amyloidogenic profiles of 34 amyloid-forming proteins generated using Cordax. The tool identifies most protein segments that were characterized as amyloidogenic during the initial collection1 of the dataset (shown in red bars) and further improves once considering recent annotations of higher accuracy (shown in magenta) (lconomidou V A et al (2013) FEBS letters 587, 569-574; Tsiolaki P et al (2015) J. of structural biology 191, 272-280; Saelices L et al (2015) The J. of Biol. Chemistry 290, 28932; Baxa U et al (2007 Biochemistry 46, 13149; Gross M et al (1999) Protein science: a publication of the protein society 8, 1350; Louros N N et al (2015) Int. J. of biological micromolecules 79, 711 and Van Melckebeke H et al (2010) J. of the American Chemical Society 132, 13765). Experimentally verified aggregation prone regions strongly predicted by Cordax are highlighted by overlaid green bars.

FIG. 8: Amyloid formation by peptides that fail to bind Thioflavin-T or pFTAA. Fibrils exhibit typical amyloid-like characteristics but appear shorter in length.

FIGS. 9A-9D: UMAP and PCA analysis of the known experimentally determined amyloidogenic sequence space. (FIG. 9A) UMAP color-coded based on predictor performances, as in FIG. 5a. (FIG. 9B) Clustering using the same basic physicochemical properties and amino acid composition scheme as in FIG. 5b. Three-dimensional principle component analysis of the amyloid sequence space color-coded based on predictor performances (FIG. 9C) and (FIG. 9D) sequence clustering indicates that Cordax infiltrates the sequence space of higher solubilities with the exception of the high disorder propensity cluster contains many false negatives.

FIGS. 10A and 10B: interaction energies of candidate capping peptides for the APR isolated from ApoA-I (SEQ ID NO: 172). The X-axis represents the cross-interaction energy and the Y-axis represents the elongation energy. Suitable Apo-AI candidate capping peptides are situated in the left-upper corner and suitable Apo-AI aggregation inducing peptides are situated in the left-lower corner.

FIG. 11: Endpoint fluorescence analysis. WT=SEQ ID NO: 172, next positions in the X-axis are SEQ ID NO: 178, 179, 180, 181, 182 and 183 which are the candidate aggregation inducing peptide variants, followed by SEQ ID NO: 173, 174, 175, 176 and 177 which are the candidate capping peptide variants.

FIGS. 12A and 12B: Th-T kinetics (performed in triplicate) for the candidate capping peptides

FIGS. 13A and 13B: Th-T kinetics (performed in triplicate) for the candidate aggregation inducing variants

### DETAILED DESCRIPTION OF THE INVENTION

The present disclosure relates generally to a machine learning engine, herein referred to as the Cordax algorithm (or in short Cordax), for the identification of amyloid core sequences present in a protein. The present disclosure also relates to a system (or apparatus) implementing the artificial intelligence (AI) platform.

Example embodiments will be described more fully hereinafter, which example embodiments are described. It should be understood that such systems, computer readable media, and methods may be embodied in many different forms and should not be construed as limited to the example embodiments set forth herein. Rather, these example embodiments are provided so that this disclosure will be thorough and complete, and will fully convey the scope of the claims to those of ordinary skill in the art.

The term “machine learning” as used herein generally refers to a type of artificial intelligence (AI) that provides computers with the ability to learn without being explicitly programmed. Machine learning is a branch of AI focusing on systems that can learn from data, identify patterns, and make decisions with minimal human intervention.

As used herein, the term “full length native protein” refers to a protein that is in its native or natural state and unaltered by any denaturing agent such as heat, chemical mutation or enzymatic reactions. A wild-type protein would be considered a full-length native protein. The term full-length native protein sequence, as used herein, refers to the amino acid sequence found in the full-length native protein.

As used herein “mutation” refers to a change in the amino acid sequence of a native protein. Mutations can be described by using the native sequence and then identifying the specific acid that have been changed. A “mutant” refers to the protein that contains the mutation. A full-length mutant sequence refers to the full amino acid sequence of the mutant protein, instead of describing the mutant as the amino acids that are different from the native protein.

Terms such as “first”, “second”, and “within” are used merely to distinguish one component (or part of a component or state of a component) from another. Such terms are not meant to denote a preference or a particular orientation and are not meant to limit embodiments of the disclosure. In the following detailed description of the example embodiments, numerous specific details are set forth in order to provide a more thorough understanding of the invention. However, it will be apparent to one of ordinary skill in the art that embodiments of the disclosure may be practiced without these specific details. In other instances, well-known features have not been described in detail to avoid unnecessarily complicating the description.

A user may be any person or entity that interacts with the database, the AI platform, or both. Examples of a user may include, but are not limited to, a principal investigator, a scientist, a post-doctoral candidate, a graduate student, or a pharmaceutical company, for example. There can be one or multiple users.

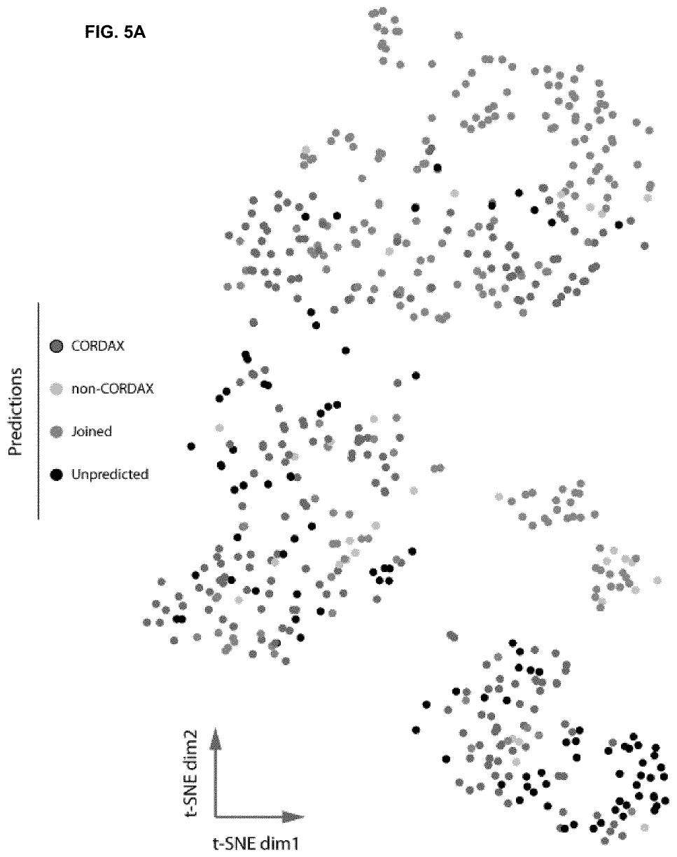

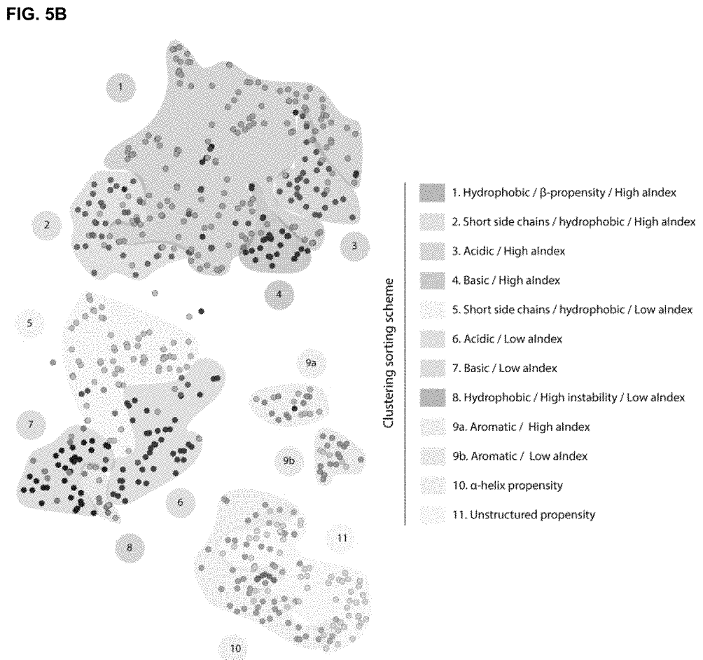

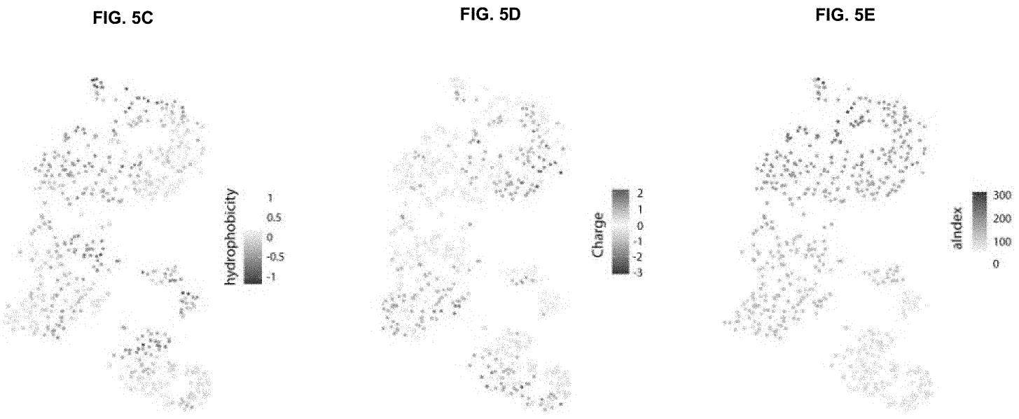

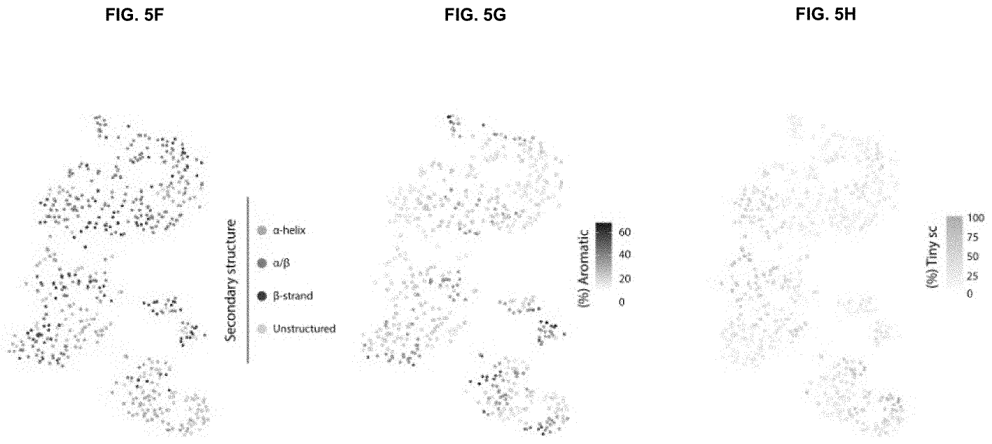

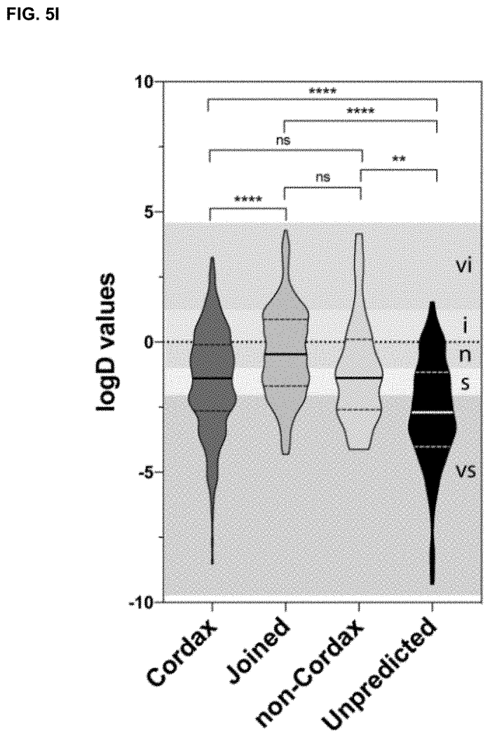

The number of amyloid structures in the protein databank has been steadily increasing over the last two decades. It has now achieved a number (>80) that was reached for globular proteins at the beginning of the 1980s and that then triggered the first developments of template-based modelling methods including homology-based and threading (or fold recognition) in an attempt to estimate the versatility of individual folds and discover novel folds in a more directed manner. In the present invention we provide a new algorithm, Cordax, which is an exhaustively trained regression model that leverages a substantial library of curated amyloid template structures combined with machine learning. Cordax uses a logistic regression approach to translate structural compatibility and interaction energies into sequence aggregation propensity and is therefore unconstrained by defined sequence tendencies, such as hydrophobicity or secondary structure preference that direct most sequence-based predictors. As a result, we have discovered unconventional amyloid-like sequences, including sequences with low aliphatic content, high net charge or sequences with low intrinsic structural propensities. Clustering amyloid sequences by t-SNE two-dimensional reduction revealed the substructure of amyloid sequence space. Apart from a large cluster corresponding to sequences found in the hydrophobic core of globular proteins, we also found clusters corresponding to surface-exposed amyloid sequences in globular proteins, small aliphatic functional amyloids, N/Q/Y prions, strongly helical and intrinsically disordered sequences which could be compatible with liquid-liquid phase responsive sequences. The present invention highlights the discovery of highly soluble, yet amyloid-forming, sequences and suggests that the largest portion of the remaining uncharted amyloid sequence space is hidden in this corner (see FIGS. 5a & 5i). Indeed, most archetypal hydrophobic APR sequences have low intrinsic solubility. As a result, low solubility and aggregation propensity are properties that are often wrongly used interchangeably. It is important to differentiate between the initial solubility and aggregation propensity of a peptide, as soluble monomeric sequences can often self-assemble, at later time points, into insoluble amyloid fibrils. The APRs that are newly discovered by Cordax are often highly soluble in their monomeric form, even more than the already known polar APRs from the yeast prions, as they contain many charged and polar residues, yet surprisingly can still assemble into amyloids. Overall, our approach demonstrates that the increasing structural information on amyloids now allows for more fine-graded structural rule learning of the amyloid state.

Cordax provides a cost-effective complementary powerful computational alternative that can be operated without any required scientific expertise necessary to apply the intricate technical approaches. Apart of its function as an aggregation predictor, the tool is uniquely poised to provide detailed complementary structural information on the putative amyloid fibril architecture of identified aggregation prone regions. Users can utilise the method to structurally characterise identified APRs by classifying their overall specific topological preferences, including β-strand directionality and key residue positions that are integral parts of the amyloid core. The latter information is imperative for efforts focused on understanding the underlying mechanisms that dictate amyloid-related diseases or the formation of functional amyloids, but can also have an immense impact on the design of applied nano-biomaterials⁶⁴, targeted amyloid inducers⁶⁵ or counteragents, following the increased interest in the development of structure-based inhibitors of aggregation⁶¹⁻⁶³.

Accordingly, the present invention provides in a first embodiment a method for identifying at least one aggregation prone region (APR) present in a protein, the method comprising:
querying a machine learning engine for a proposed APR present in a protein, wherein the machine learning engine was trained using a first library comprising experimentally defined amyloidogenic sequences from amyloid-forming proteins wherein said amyloidogenic sequences were modelled on the backbone structures of a second library of amyloid fibril core structures and wherein the thermodynamic stability of each model was calculated by a Force Field and said calculations were introduced into a logistic regression model to score the aggregation propensity and, obtaining at least one candidate APR sequence.

In a specific embodiment the querying of the machine learning engine (or algorithm which is an equivalent word) involves fragmenting said protein into hexapeptides using a sliding window process, followed by modelling said hexapeptides on the backbone of said second library, calculating the thermodynamic stability for each sequence using a Force Field and feeding the data into said logistic regression model.

In a specific embodiment the Force Field used is FoldX.

In specific embodiments the invention provides a computer-readable storage medium which stores computer-executable instructions that, when executed by at least one processor, cause the processor to perform one of the methods described herein before in the embodiments.

In yet another embodiment the invention provides an apparatus comprising control circuitry configured to perform one of the methods described in the previous embodiments.

Systems of the disclosure can include an intranet-based computer system that is capable of communicating with various software. A computer system includes any type of computing device or communication device. Examples of such a system can include, but are not limited to, super computers, a processor array, distributed parallel system, a desktop computer with LAN, WAN, Internet or intranet access, a laptop computer with LAN, WAN, Internet or intranet access, a smart phone, a server, a server farm, an android device (or equivalent), a tablet, smartphones, and a personal digital assistant (PDA). Further, as discussed above, such a system can have corresponding software (e.g. user software, sensor device software). The software of one system can be a part of, or operate separately but in conjunction with, the software of another system.

Embodiments of the disclosure include a storage repository. The storage repository can be a persistent storage device (or set of devices) that stores software and data. Examples of a storage repository can include, but are not limited to, a hard drive, flash memory, some other form of solid-state data storage, or any suitable combination thereof. The storage repository can be located on multiple physical machines, each storing all or a portion of the database, AI platform, protocols, algorithms, or other stored data according to some example embodiments. Each storage unit or device can be physically located in the same or in a different geographic location. In embodiments, the storage repository may be stored locally, or on cloud-based serveries such as Amazon Web Services.

In one or more example embodiments, the storage repository stores one or more databases, AI Platforms, protocols, algorithms, and stored data. The protocols can include any of a number of communication protocols that are used to send, receive, or send and receive data between the processor, datastore, memory and the user. A protocol can be used for wired and/or wireless communication. Examples of a protocols can include, but are not limited to, Modbus, profibus, Ethernet, and fiberoptic.

Systems of the disclosure can include a hardware processor. The processor of the executes software, algorithms, and firmware in accordance with one or more example embodiments. The processor can be a central processing unit, a multi-core processing chip, SoC, a multi-chip module including multiple multi-core processing chips, or other hardware processor in one or more example embodiments. The processor is known by other names, including but not limited to a computer processor, a microprocessor, and a multi-core processor. The processor can also be an array of processors.

In one or more example embodiments, the processor executes software instructions stored in memory. Such software instructions can include generating machine learning models, executing machine learning models, performing analysis on data received from the database, and so forth. The memory includes one or more cache memories, main memory, or any other suitable type of memory. The memory can include volatile or non-volatile memory.

The processing system can be in communication with a computerized data storage system which can be stored in the storage repository. The data storage system can include a non-relational or relational data store, such as a MySQL or other relational database. Other physical and logical database types could be used. The data store may be a database server, such as Microsoft SQL Server., Oracle., IBM DB2., SQLITE., or any other database software, relational or otherwise. The data store may store the information identifying syntactical tags and any information required to operate on syntactical tags. In some embodiments, the processing system may use object-oriented programming and may store data in objects. In these embodiments, the processing system may use an object-relational mapper (ORM) to store the data objects in a relational database. The systems and methods described herein can be implemented using any number of physical data models. In one example embodiment, an RDBMS can be used. In those embodiments, tables in the RDBMS can include columns that represent coordinates. The tables can have pre-defined relationships between them. The tables can also have adjuncts associated with the coordinates.

In embodiments, the systems of the disclosure can include one or more I/O (input/output) devices allow a user to enter commands and information into the system, and also allow information to be presented to the user or other components or devices. Examples of input devices include, but are not limited to, a keyboard, a cursor control device (such as a mouse), a microphone, a touchscreen, and a scanner. Examples of output devices include, but are not limited to, a display device (e.g., a display, a monitor, or projector), speakers, outputs to a lighting network (such as a DMX card), a printer, and a network card. For example, the input devices can be used to enter data on native proteins and mutation sequences and assays. The input devices can also enter wanted functional data for a protein. The output devices can be used to output analysis data and/or engineered protein sequences resulting from AI protein design.

Various techniques are described herein in the general context of software.

Generally, software includes routines, programs, objects, components, data structures, and so forth that perform particular tasks or implement particular abstract data types. An implementation of these modules and techniques can be stored on or transmitted across some form of computer readable media. Computer readable media is any available non-transitory medium or non-transitory media that is accessible by a computing device. By way of example, and not limitation, computer readable media includes computer storage media.

In embodiments, the AI Platform comprises a machine learning method, such as a neural network for effective protein function prediction. In some embodiments, the AI platform includes neural networks, genetic algorithms, decision trees, fuzzy logic, symbolic rules, gradient boosting, support vector machines, and other machine learning based systems. Pluralities and/or combinations of the above may also be used. In embodiments, the AI Platform can use ML frameworks such as, Keras, Caffe, Pytorch, TensorFlow, the Microsoft Cognitive Toolkit, MXNet, Chainer, and Theano, with a Python implementation as the predominant data science language. In embodiments, the AI platform will allow for agnostic integration with other algorithms (such as gradient boosting, SVM, Gaussian processes) and their respective frameworks (XGBoost, SciKit Learn, GPy etc.) by separating data preparation from model creation and by using a NumPy data format common to all of these frameworks. In some embodiments, data preparation tools can be released as a Python package.

Embodiments of the disclosure use protein feature encodings to add physical or biological knowledge to amino acid sequences to create representations amenable to machine learning. As the choice of encoding varies based on the size and diversity of the input, as well as the task, several encoding methods can be implemented, allowing users to test and select the encodings most relevant to their problem. The AI Platform can include the following encodings, for example: one-hot, autoencoders, amino acid property encoders, learned BLOSUM/MSA evolutionary encodings, sequence mutation representation relative to WT, secondary structure/solvent accessible surface area encodings, learned AA embeddings, POOL, Phoenix, and/or structural/graph/topological encodings.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

One or more processors may be interconnected by one or more networks in any suitable form, including as a local area network or a wide area network, such as an enterprise network or the Internet. Such networks may be based on any suitable technology and may operate according to any suitable protocol and may include wireless networks, wired networks, or fiber optic networks.

One or more algorithms for controlling methods or processes provided herein may be embodied as a readable storage medium (or multiple readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible storage medium) encoded with one or more programs that, when executed on one or more computers or other processors, perform methods that implement the various methods or processes described herein.

In some embodiments, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the methods or processes described herein. As used herein, the term “computer-readable storage medium” encompasses only a computer-readable medium that can be considered to be a manufacture (e.g., article of manufacture) or a machine. Alternatively or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computer or other processor to implement various aspects of the methods or processes described herein. Additionally, it should be appreciated that according to one aspect of this embodiment, one or more programs that when executed perform a method or process described herein need not reside on a single computer or processor, but may be distributed in a modular fashion amongst a number of different computers or processors to implement various procedures or operations.

Executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. Non-limiting examples of data storage include structured, unstructured, localized, distributed, short-term and/or long term storage. Non-limiting examples of protocols that can be used for communicating data include proprietary and/or industry standard protocols (e.g., HTTP, HTML, XML, JSON, SQL, web services, text, spreadsheets, etc., or any combination thereof). For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags, or other mechanisms that establish relationship between data elements.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used, Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one refers, whether related or unrelated to those elements specifically identified. Thus, as a non-limiting example, “at least one of A and B” (or, equivalently, at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of and “consisting essentially of shall be closed or semi-closed transitional phrases, respectively, as set forth in the United States Patent Office Manual of Patent Examining Procedures, Section 2111.03.

### Examples

1. General Overview of the Cordax Algorithm

In the present invention we have designed a novel structure-based amyloid core sequence prediction method that (a) leverages all the available structure information that is currently available, and (b) employs a machine learning element for optimal prediction performance. In a first step a curated template library of amyloid core structures as described was built (see the Cordax library described in example 2 below). Similar to known prediction methods″, we fixed on the hexapeptide as a unit of prediction. In order to determine the amyloid propensity of a query hexapeptide we start by modelling its side chains on all the available amyloid template structures using the FoldX force field³⁰, which yields a model and an associated free energy estimate (DeltaG, kcal/mol) for each template. These free energies are then fed into a logistic regression model (see example 3), which is a simple statistical method relating a binary outcome to continuous variables. The prediction output of Cordax is multiple: first, there is the prediction from the logistic regression whether or not the segment is an amyloid core sequence, second, for the sequences predicted to be an amyloid core, the most likely amyloid core model is provided. For longer query sequences, a sliding window approach is adopted. Specific technical details of the pipeline are outlined below in the further examples.

2. Collection, Refinement and Characterisation of Fibril Structures for Machine Learning, Building of the Cordax Library

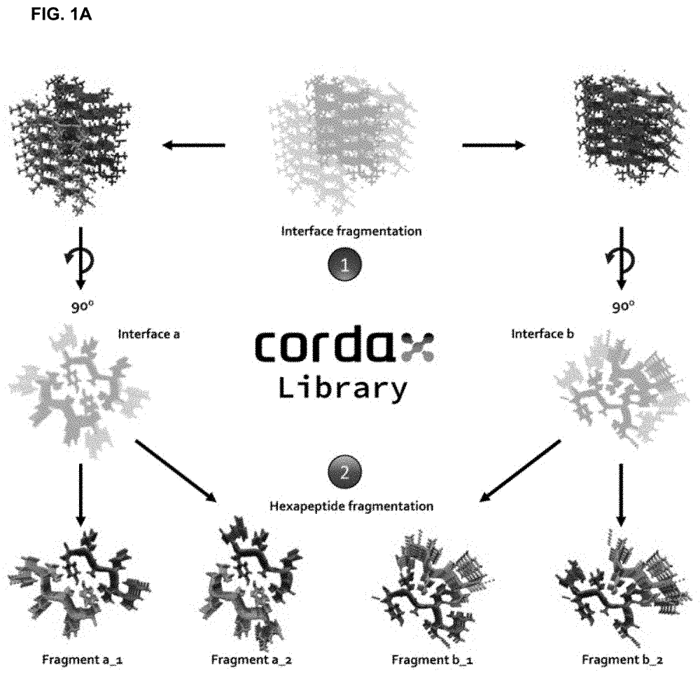

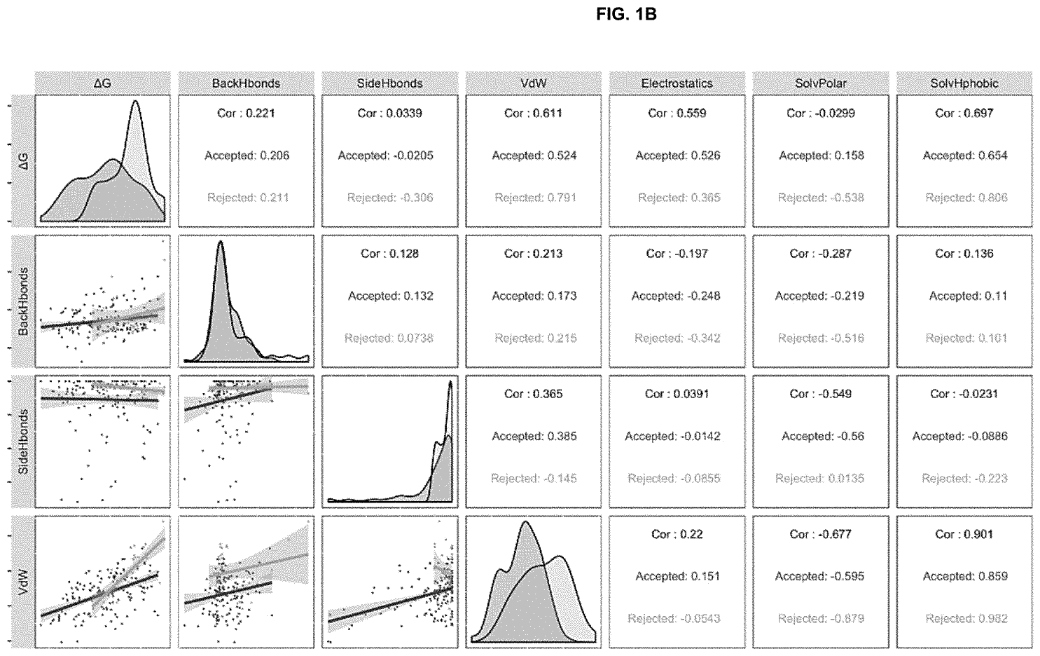

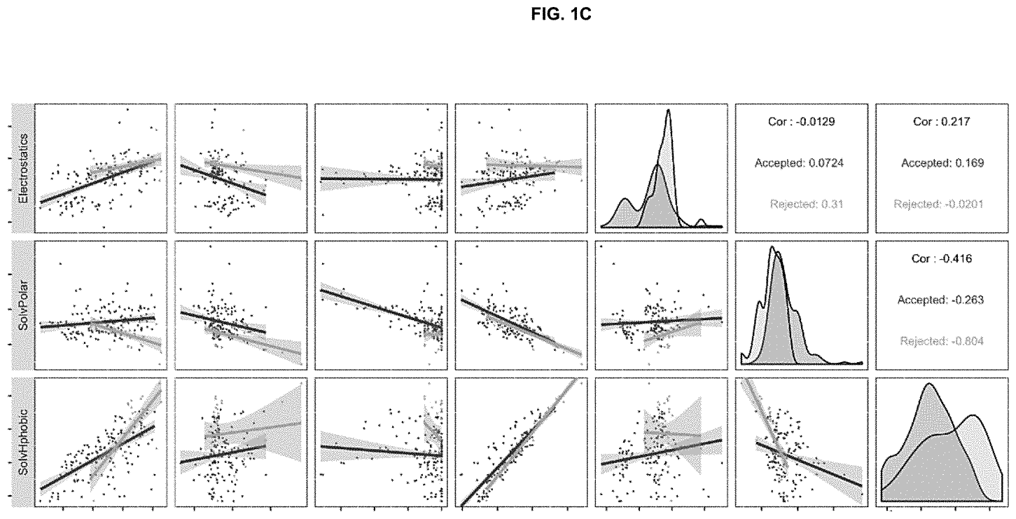

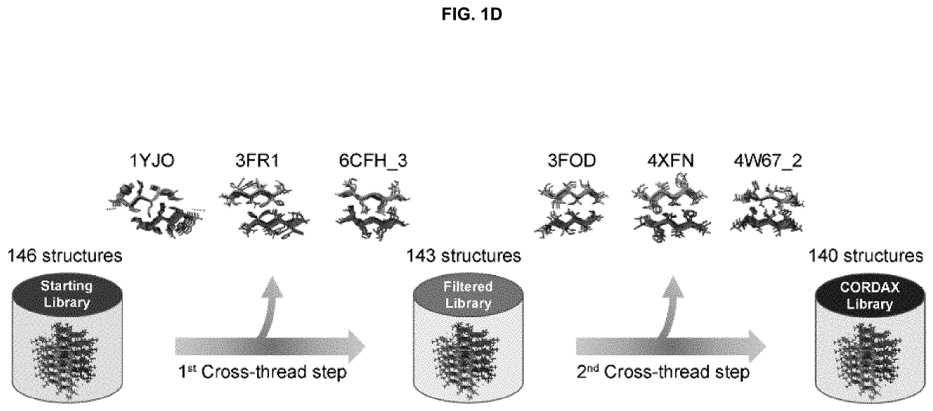

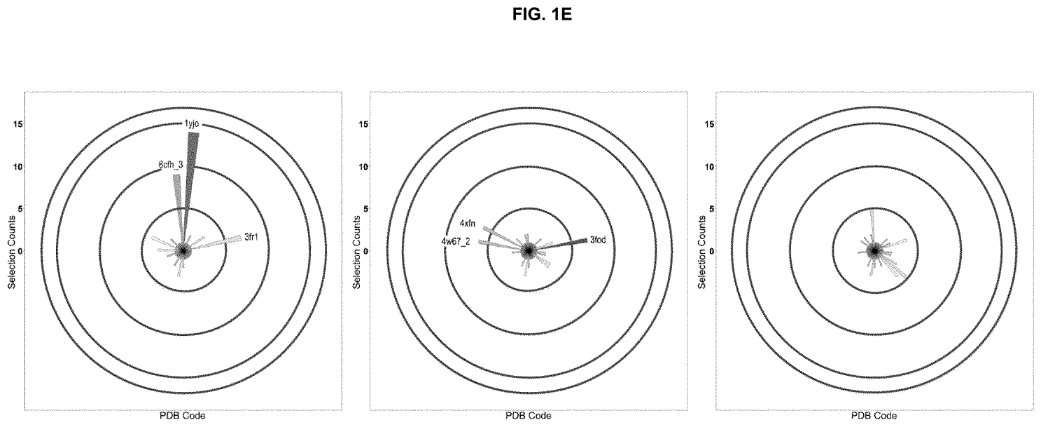

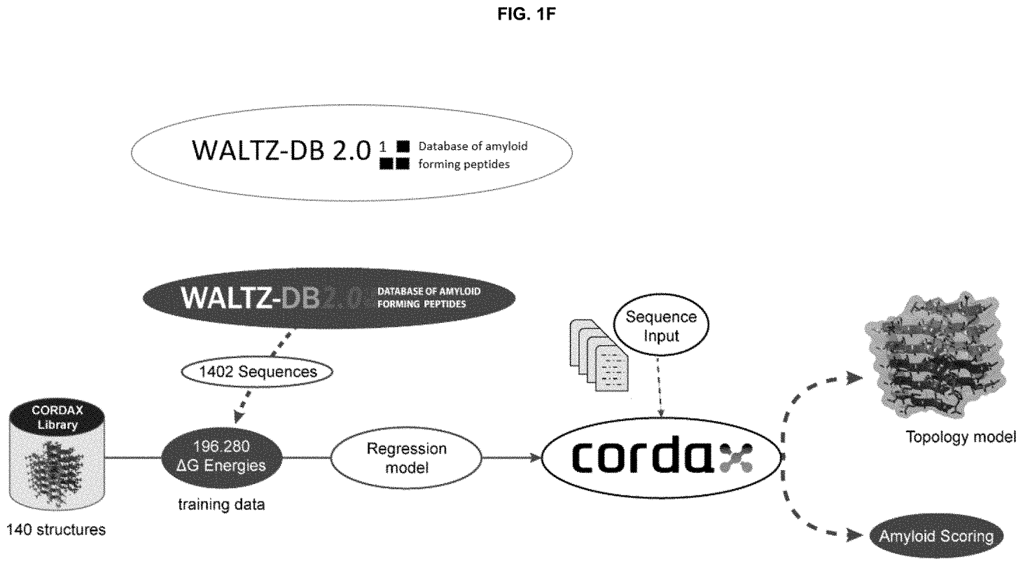

We isolated 78 short segment fibril core high resolution structures from the Protein Data Bank (see Table 1). Templates were grouped into 7 distinct topological classes out of 8 theoretically possible based on their overall structural properties, as previously proposed by Sawaya et al³¹. Briefly, topologies are defined by whether β-sheets have parallel versus antiparallel orientation, by the orientation of the strand faces that form the steric zipper (face-to-face versus face-to-back), and finally the orientation of both sheets towards each other and whether that results in identical or different fibril edges. This complexity was addressed by generating an ensemble of amyloid cores per structure using crystal contact information derived from the solved structures. Every template comprises two facing β-sheets, each composed of five successive β-strands. Since parallel architectures can share more than one homotypic packing interface, those structures were split into separate individual entries (FIG. 1a). To ensure uniformity, we expanded the number of structural variants by breaking down longer segments into hexapeptide constituents, thus yielding a library of 179 peptide fragment structures (FIG. 1a & Table 1).

The amyloid interaction interfaces were analysed in detail following energy refinement by the FoldX force field³⁰. During this step we identified and rejected 33 imperfect β-packing interfaces formed by β-strands that contribute less than three interacting residues, thus reducing the ensemble to 146 structures. Detailed analysis of the contributions of various energy components showed that these excluded β-packing interfaces have inefficient shape complementarity and low overall stability, stemming from a combination of weak electrostatic contributions, diminished van der Waals interactions and exposure of hydrophobic residues to the solvent (FIGS. 1b and 1c). Previous work has highlighted that distinct topological layouts can potentially introduce a stronger tolerance for the integration of protein sequence segments and as a result can generate several potential type-I errors (false positives)²⁹. To address this issue, we implemented a two-step cross-threading exploration of putative structural promiscuous traps. In more detail, we extracted a non-redundant set of hexapeptide sequences from the structural library (73 sequences), which was subsequently cross-modelled in an all-against-all reiteration process. Using an empirical cut-off threshold (=5), a sum of 3 structural fragments was initially identified and removed. Eliminating these structures led to the identification and subsequent elimination of three additional promiscuous templates, resulting in the final Cordax library, composed of 140 zipper structures (FIGS. 1d-1f & Table 1).

3. Regression Model Training Using Peptide Sequences with Experimentally Determined Amyloid-Forming Properties

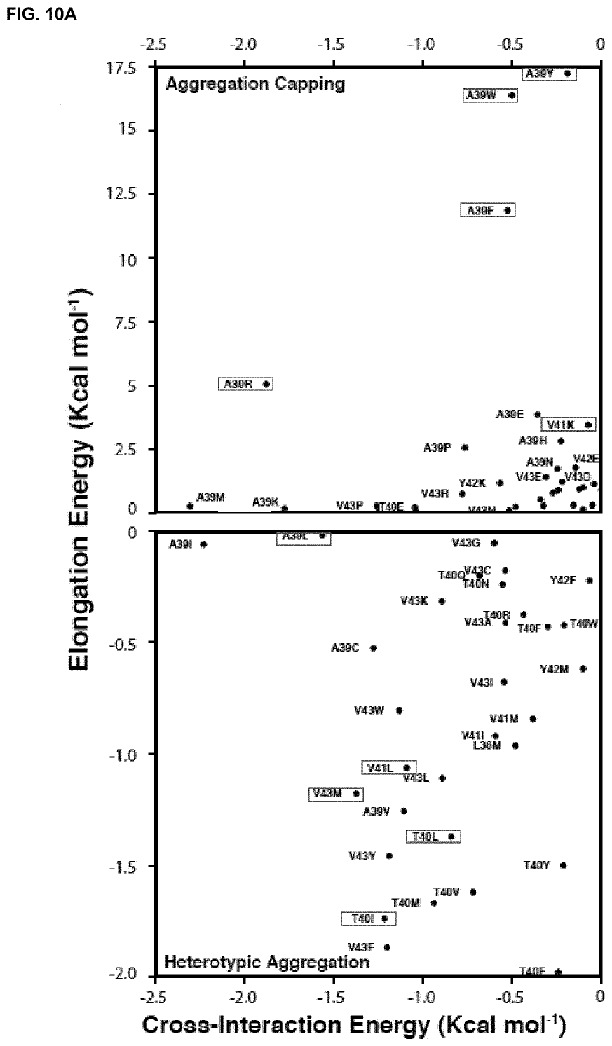

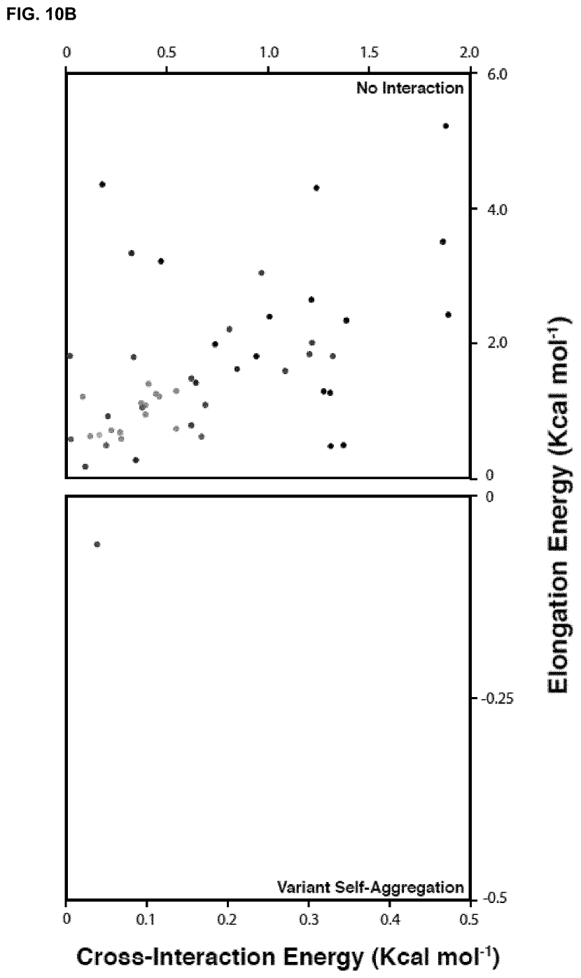

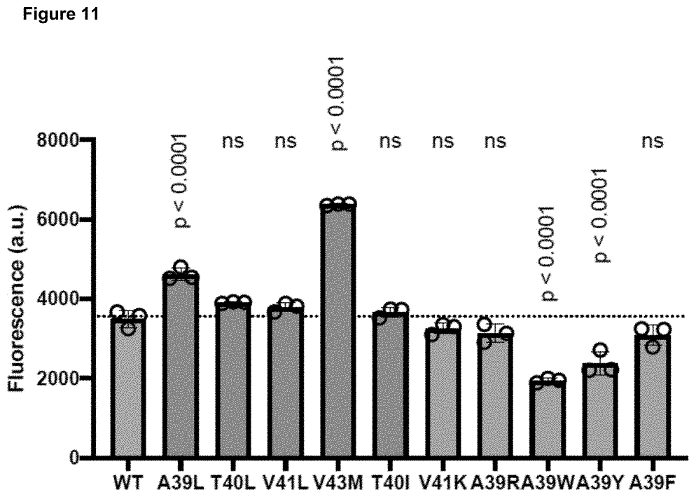

In previous work we synthesised and explored the aggregation potential of 940 peptide sequences derived from both functional and pathological amyloid-forming proteins, which were supplemented with additional data on 462 hexapeptides derived from other published sources to develop WALTZ-DB 2.0³², the largest public comprehensive repository of experimentally defined amyloidogenic peptides. In total, 1402 hexapeptide sequences from WALTZ-DB were modelled on the 140 backbone structures of the Cordax library, leading to the generation of 196280 models. The thermodynamic stability of each model (ΔG, kcal/mol) was calculated using FoldX and fed into a logistic regression model (FIG. 10. This model was used to distil the aggregation propensity from the free energy values. Towards this end, from the calculated ΔGs, we isolated 50 representative energies using a recursive feature elimination algorithm (using the RFE module of the SciKit-learn python package³³ and selecting for the set of templates that maximized the AUC). As a result, each sequence is described with a 50-dimensional vector. Next, the data were transformed in order to be constrained in a scoring range between 0 and 1, using a Min/Max scaling algorithm. The regression model was trained with L2 penalty and regularisation strength (C) equal to 1. Both scaling of the estimated ΔG and the machine learning model were developed using the SciKit-learn python package⁶⁶.

4. Benchmarking Peptide and Regional Detection of Aggregation Propensity with the Cordax Algorithm

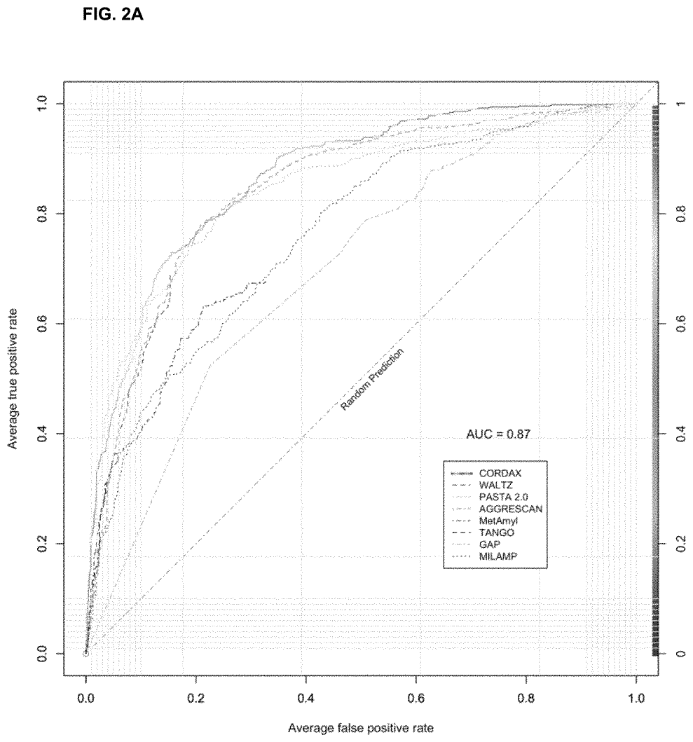

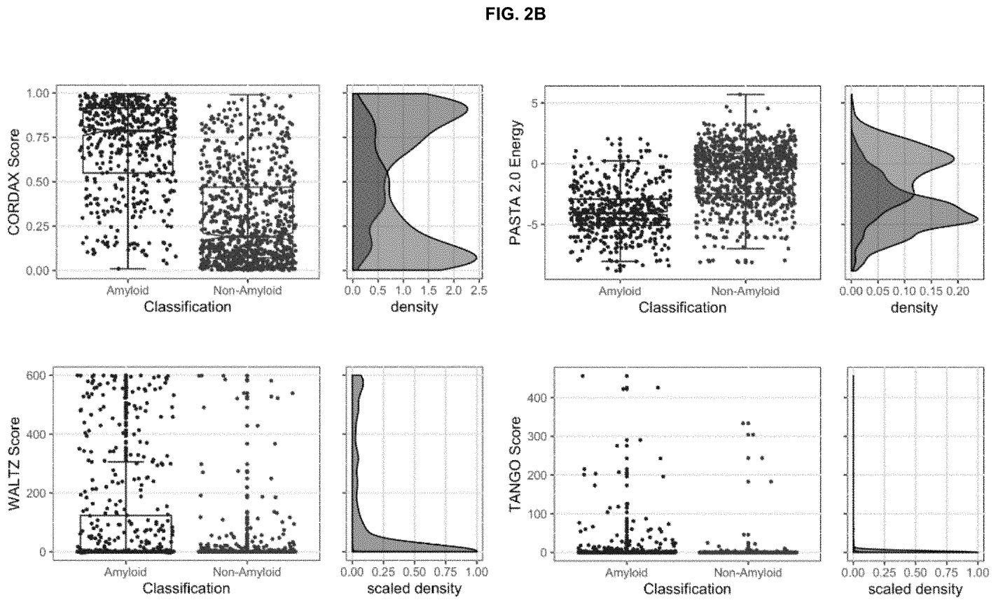

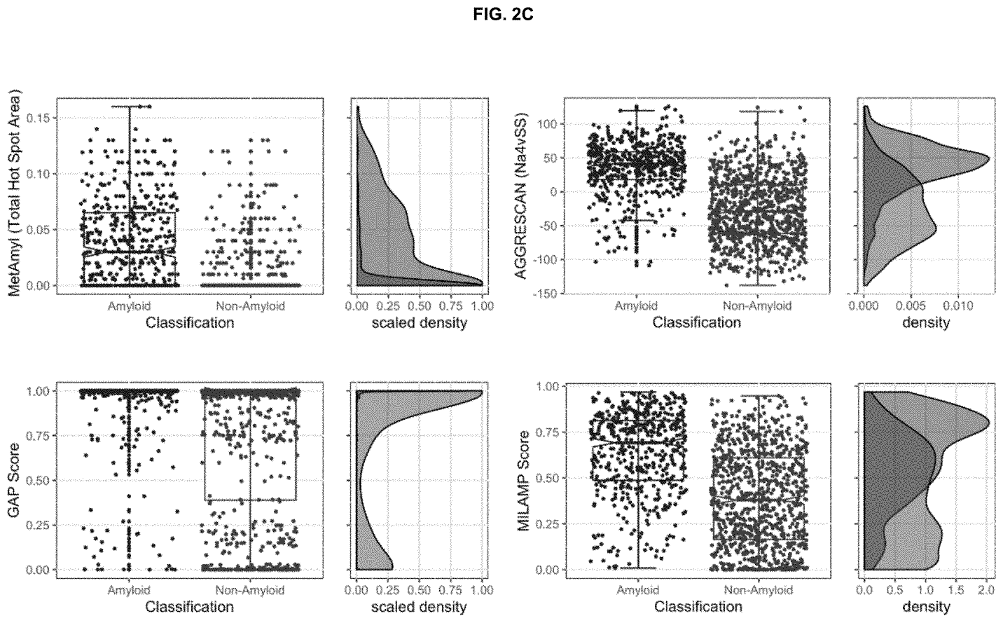

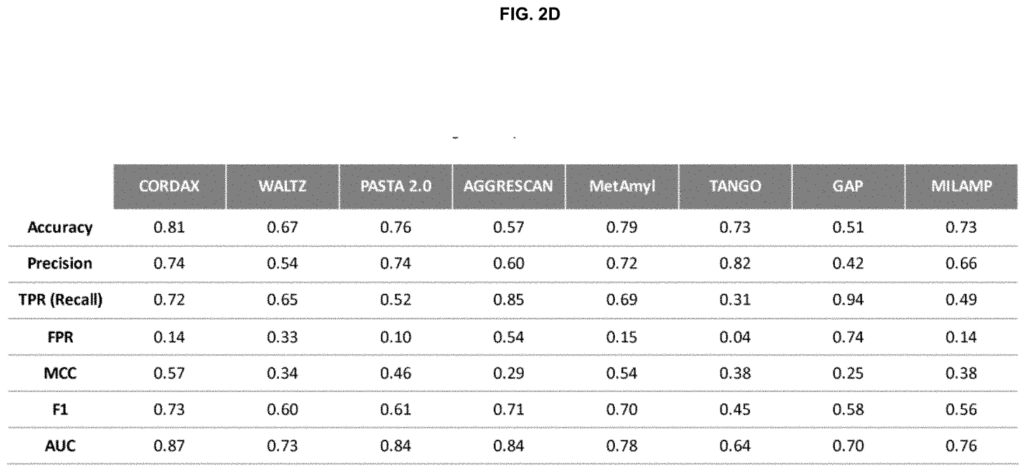

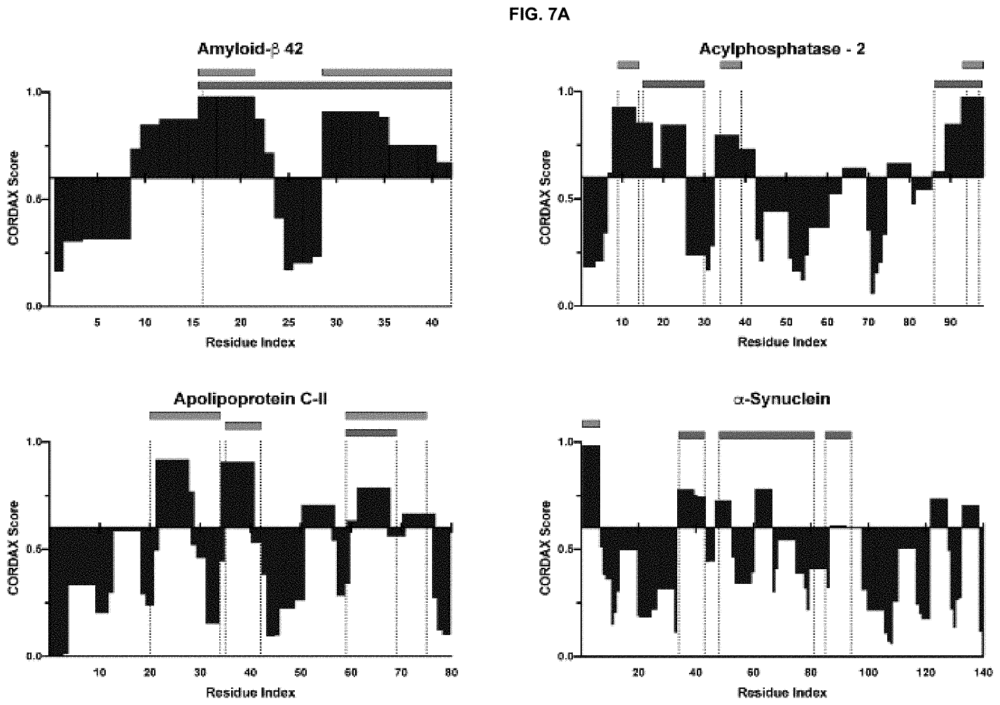

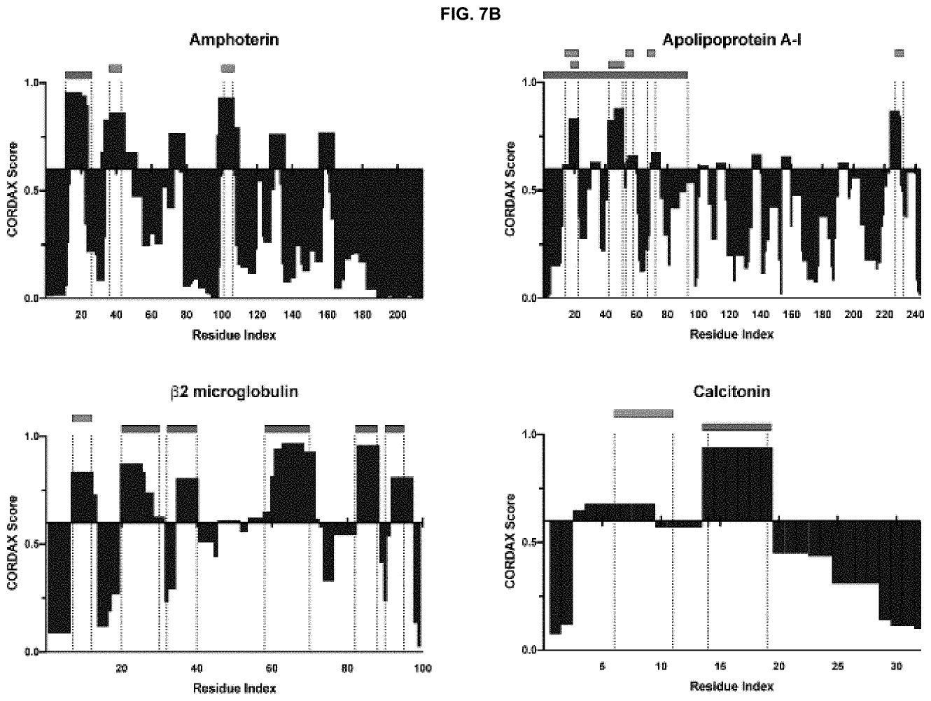

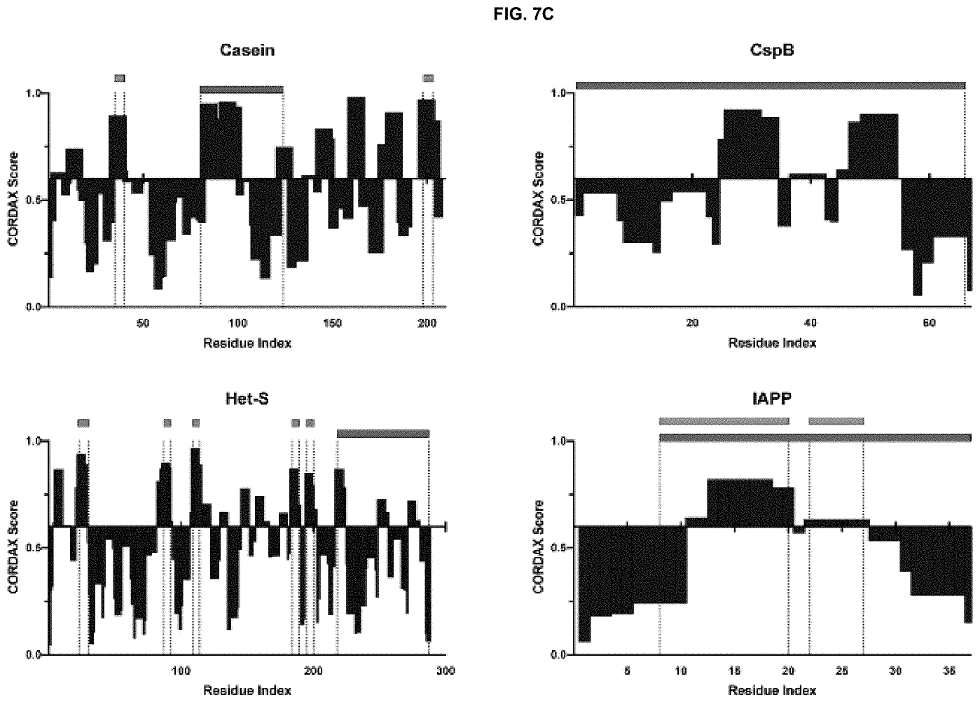

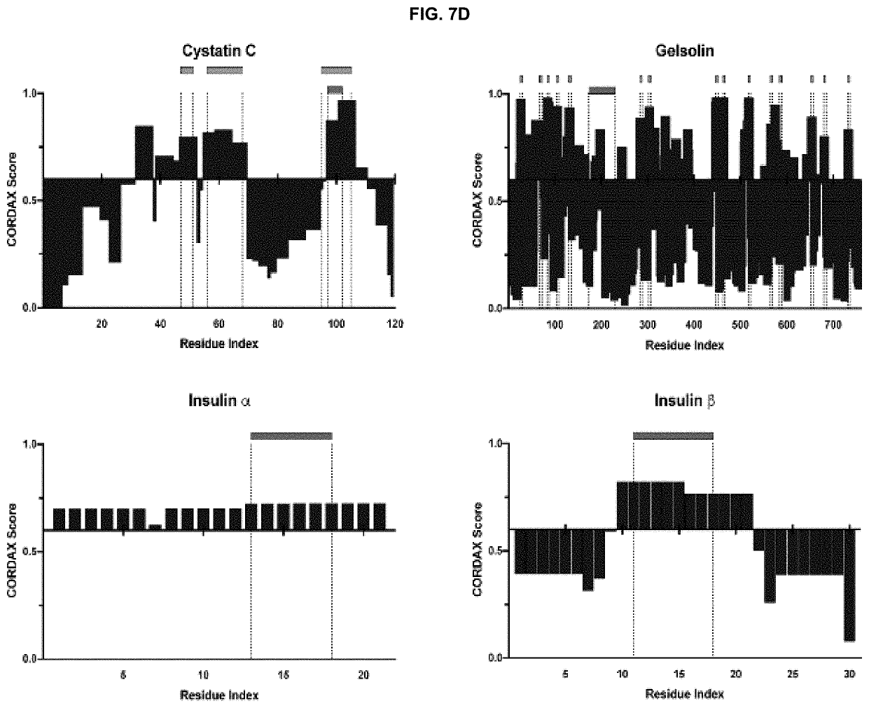

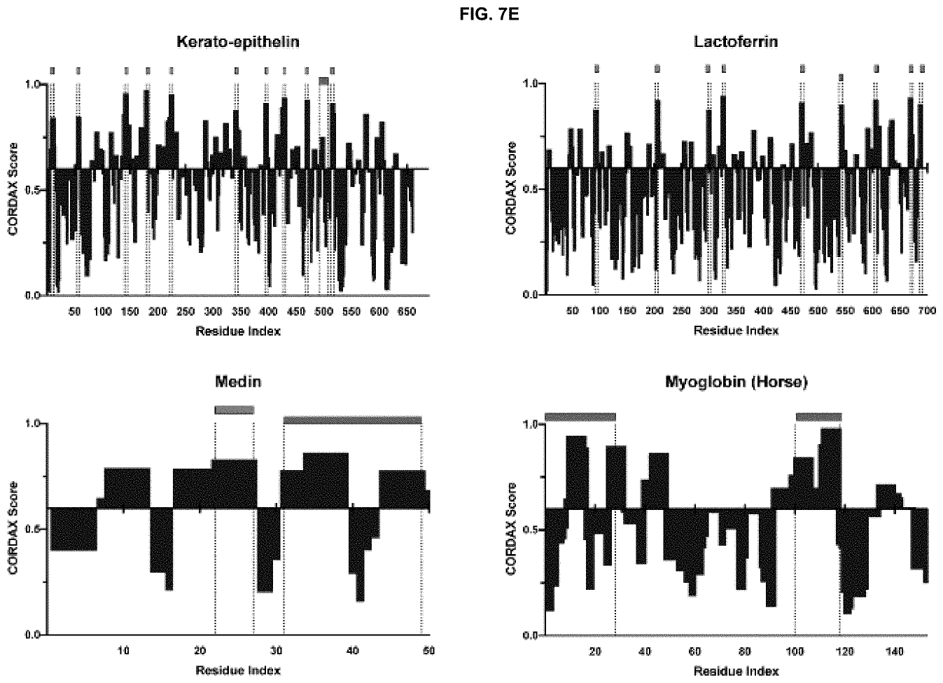

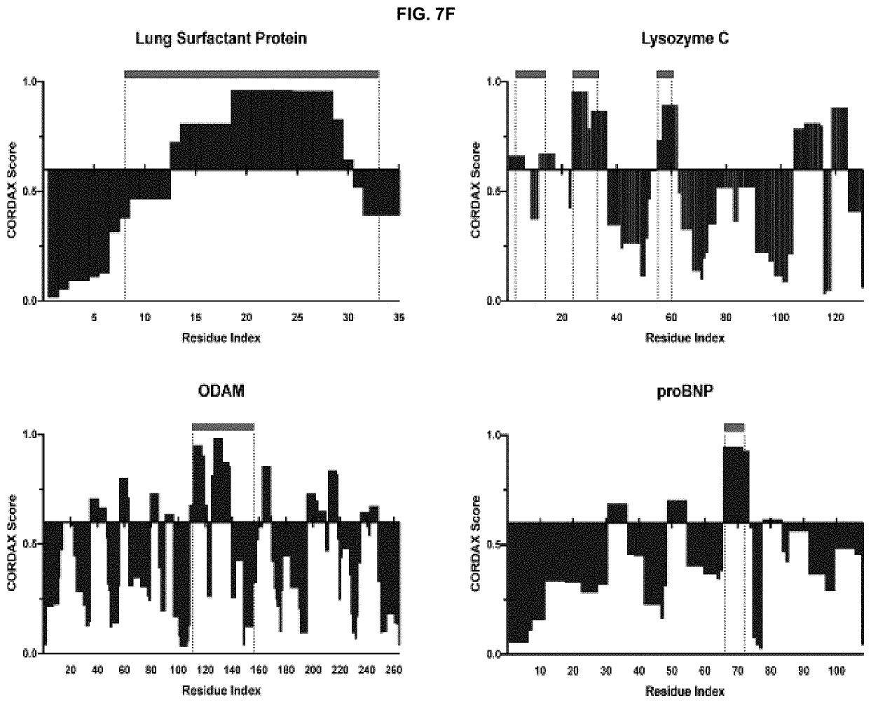

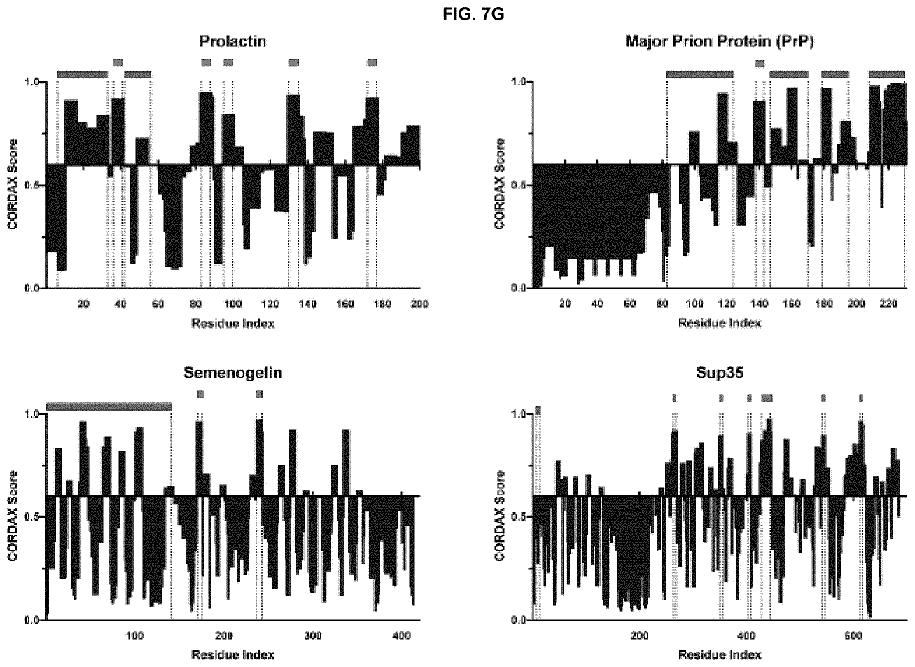

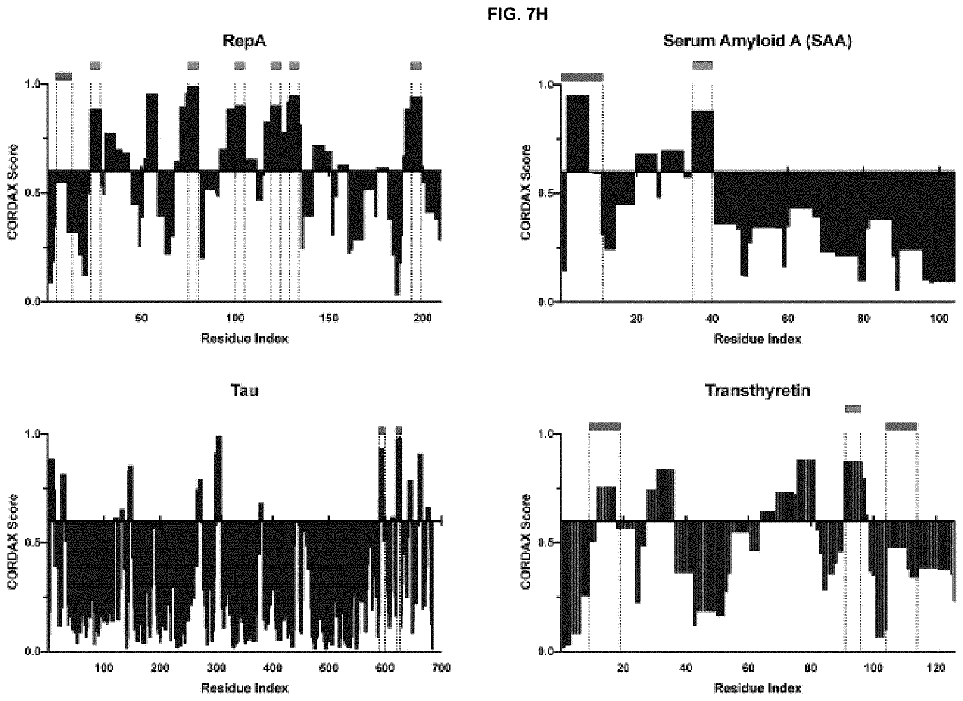

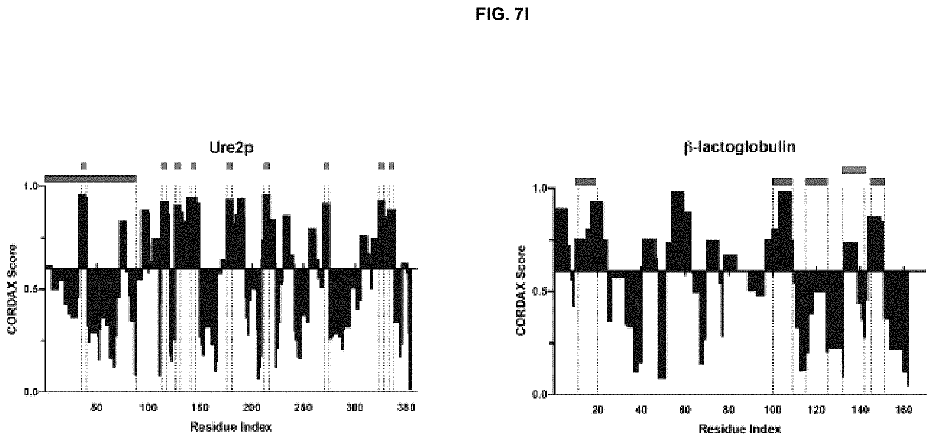

As an initial test of the prediction accuracy of the regression model, we performed leave-one-out cross-validation on the training dataset³² and performance metrics were determined on a peptide basis. Due to the extensive size of the dataset, comparison to other software was performed only with methods supporting multiple sequence input and a non-binary scoring function, since performances were compared using Receiver Operating Characteristic (ROC) analysis³³. The ROC curves generated highlight that Cordax performance exceeds over 8 state-of-the-art methods, which we applied using optimised options defined by the developers^{7,9,21-24,34.} In detail, Cordax performs well over random as depicted by the highest total area under the curve (AUC) value of 0.87 (FIG. 2a). Distribution analysis of the scoring values indicates that the method achieves optimal separation, resulting in minimal scoring overlay between positive and negative amyloid forming sequences (FIGS. 2b and 2c). As previously reported, TANGO showed high specificity due to the overrepresentation of unscored values, which is also evident for WALTZ as well as MetAmyl, which incorporates the latter method in its meta-prediction. The cost of high specificity is also reflected by the calculated F1 values, as PASTA and TANGO report low recall values. On the other hand, AGGRESCAN and GAP produce significant overpredictions as depicted by their reported false positive rates (FPR values of 0.54 and 0.76, respectively) (FIG. 2d). The optimal score thresholding of our method was determined from the ROC curve analysis as the score where predictions show the highest sensitivity-to-specificity ratio. According to this, Cordax achieves a well-balanced prediction by reporting with high specificity (86%) more than 7 out of 10 aggregation prone segments (72%), which is reflected by the highest calculated MCC, AUC and F1 values compared to other available software (FIG. 2d). To further benchmark the method, we tested it against full-length protein sequences. For this we used a standardised set of 34 annotated amyloidogenic proteins that was previously implemented for validation of several previous aggregation predictors″, following a filtering step for potential overlaps to the training data set. Despite its wide use, this collection suffers from insufficient experimental characterisation of certain large entries (i.e. gelsolin, kerato-epithelin, lactoferrin, amphoterin and others), which has been shown to introduce type-I errors (false positives). This error propensity derives from non-amyloid annotations which primarily correspond to regions of undetermined aggregation propensity, a notion that is highlighted by recent studies, such as in the case of calcitonin³⁵, cystatin-C³⁶ and transthyretin³⁷. In contrast, other proteins have been linked to the formation of β-helical structures and as an aftereffect contain elongated fragments characterised, yet unverified in their entirety, as amyloidogenic, which can introduce type-II errors (false negatives) when applying predictors of local aggregation propensity³⁸⁻⁴¹. The aforementioned shortcomings are reflected by the low MCC values that are reported for all aggregation predictors (Table 5) and the fact that predicted segments were originally considered neutral, but later shown to be aggregation hotspots (see FIGS. 7a-7i)³⁵⁻⁴¹.

5. Designed Aggregation Prone Peptide Nucleators Validate the Accuracy of Cordax Algorithm Predictions

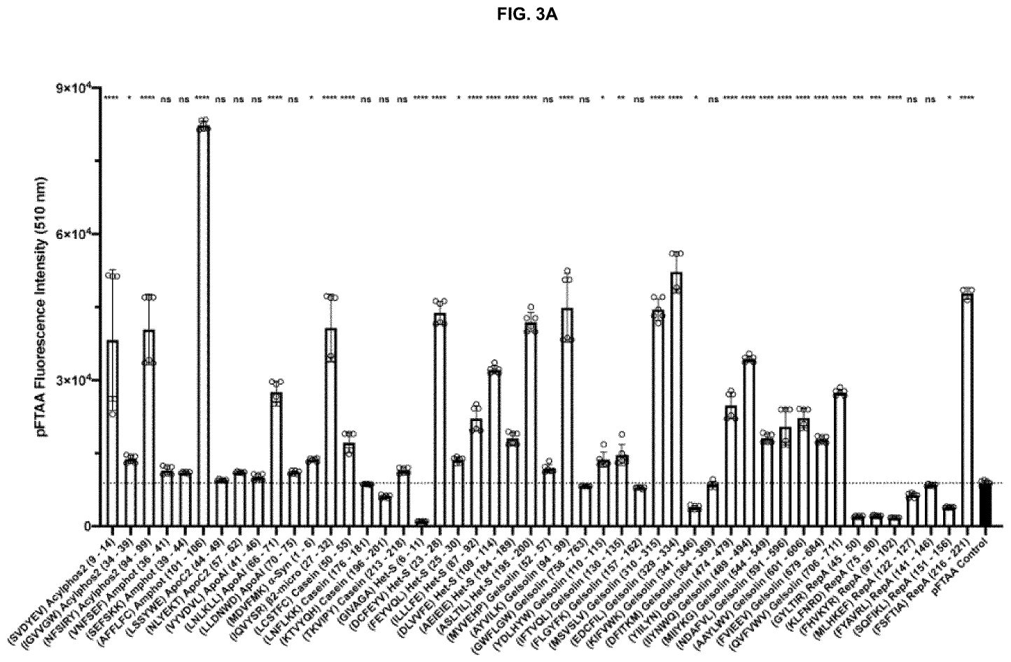

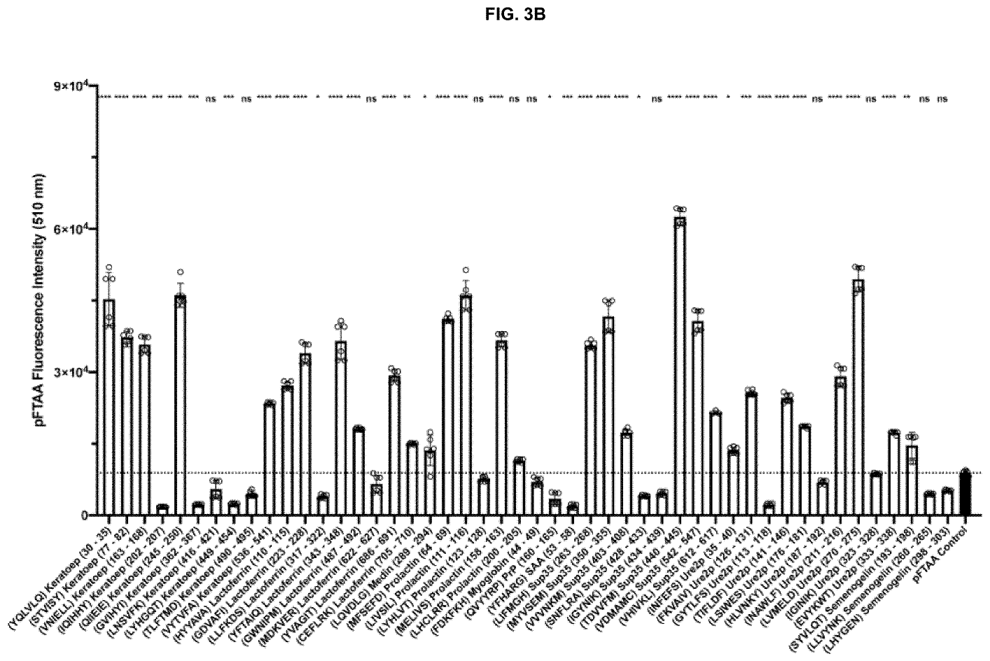

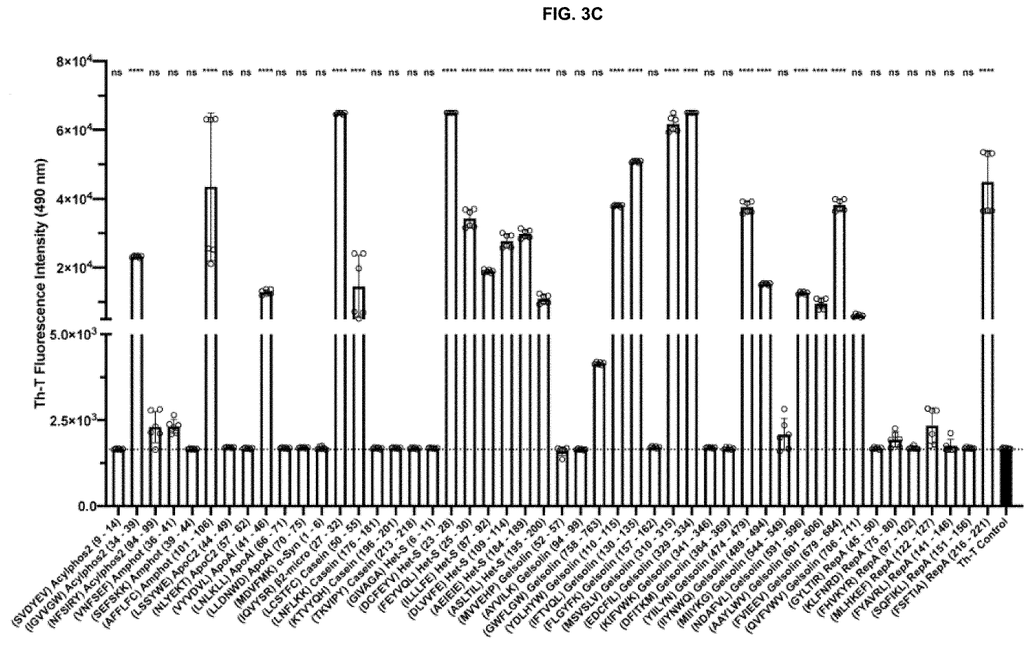

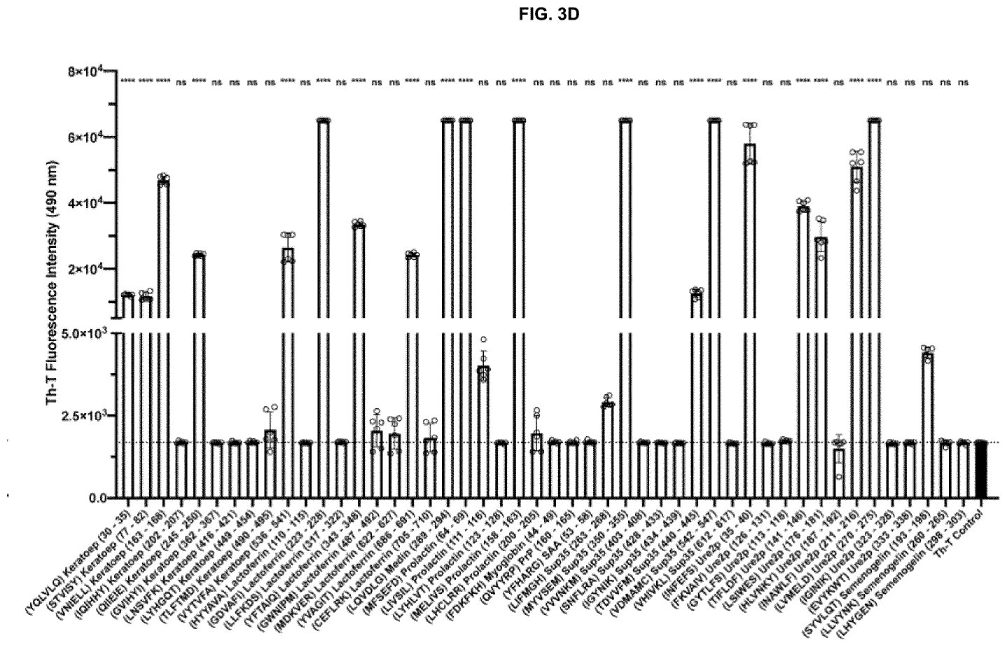

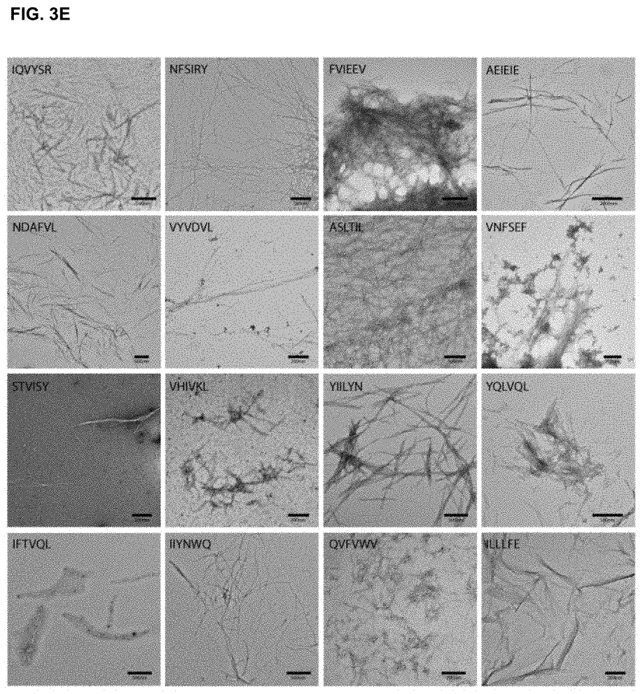

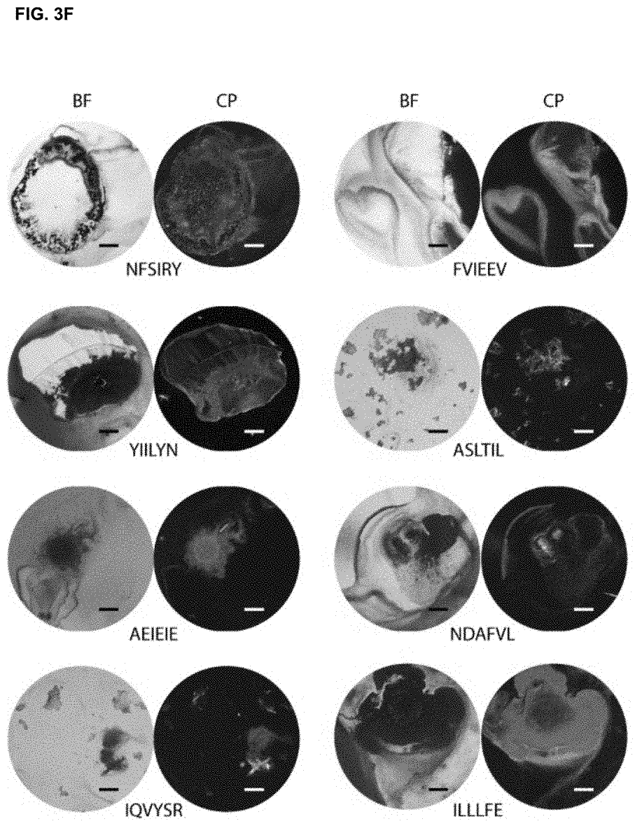

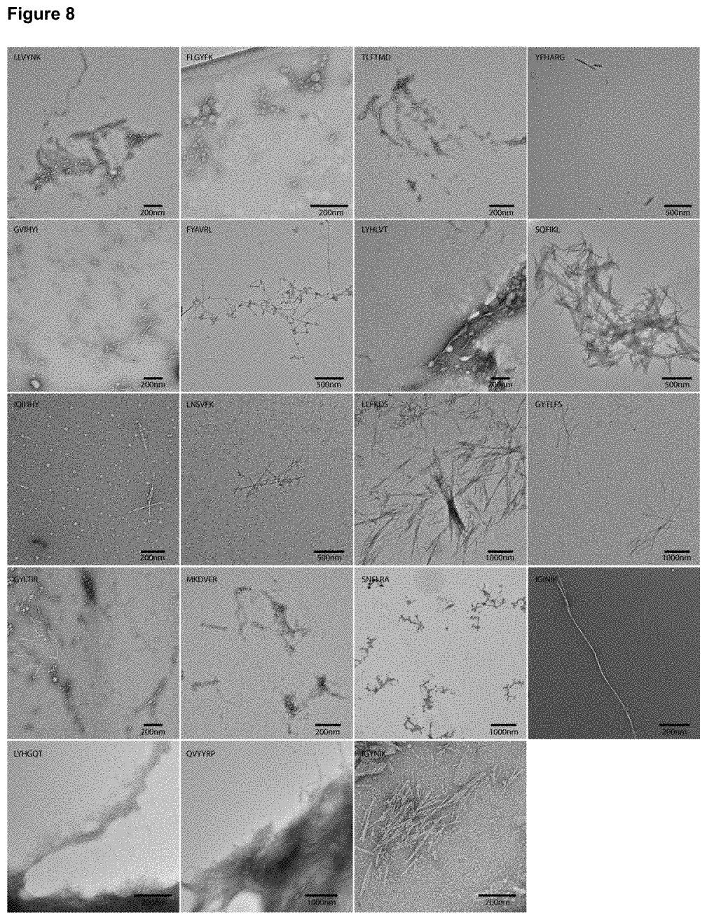

In the interest of improving the current description of the familiar amyloidogenic protein dataset, we selected and synthesised a subset of 96 peptides corresponding to strong aggregation prone regions identified in these proteins by Cordax. Apart of prediction strength, the peptide screen was also selectively constructed to ensure broad sequence variability and a wide distribution on the proteins of the dataset, with a preference for longer entries defined by inadequate previous characterisation. Peptide sequences were cross-checked and filtered to exclude overlapping sequences with previously identified amyloid regions and WALTZ-DB (see Table 2). The remaining selection of 96 peptides were synthesized using standard solid phase synthesis and their amyloid-forming properties were initially examined using Thioflavin-T (Th-T) or pFTAA binding, following rotating incubation for 5 days at room temperature. The binding assays are complementary, as Th-T and pFTAA are opposingly charged molecules, which increases the amyloid identification rate by overcoming cases of dye-specific failure to bind to amyloid surfaces based on charge repulsion. Under these conditions, 66 peptides successfully bind the specific dyes (FIGS. 3a & 3b) by forming fibrils with typical amyloid morphologies and properties that were verified using transmission electron microscopy (FIG. 3c) and Congo red staining for selected cases (FIG. 3d). As these dyes are known to yield false negatives, in particular for short peptides, all dye-negative peptides were further investigated using electron microscopy. During this scan, we recovered 19 additional sequences that were capable of forming sparse amyloid-like fibrils with shorter lengths (see FIG. 8). Taking the latter into account, Cordax was able to fish out a total number of 85 novel nucleation segments with unparalleled accuracy (89%), thus providing a rigorously improved description of the protein set to be used for the efficient testing and development of future predictors (see FIGS. 7a-7i).

6. Machine-Guided Structural Prediction Detects Highly Soluble Surface-Exposed Conformational Switches of Aggregation

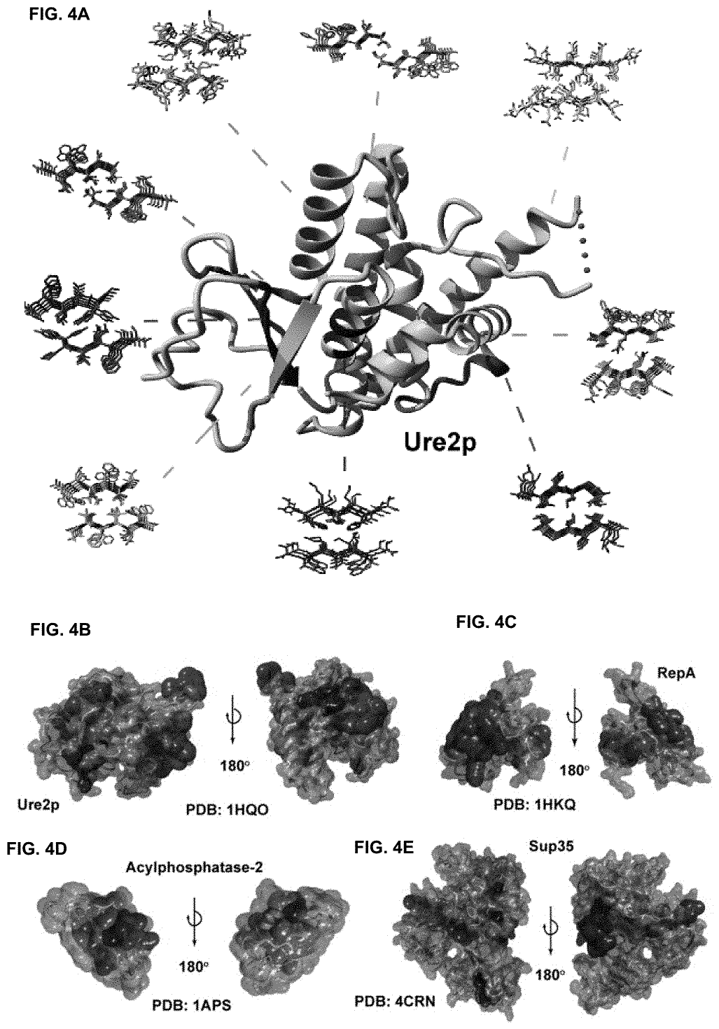

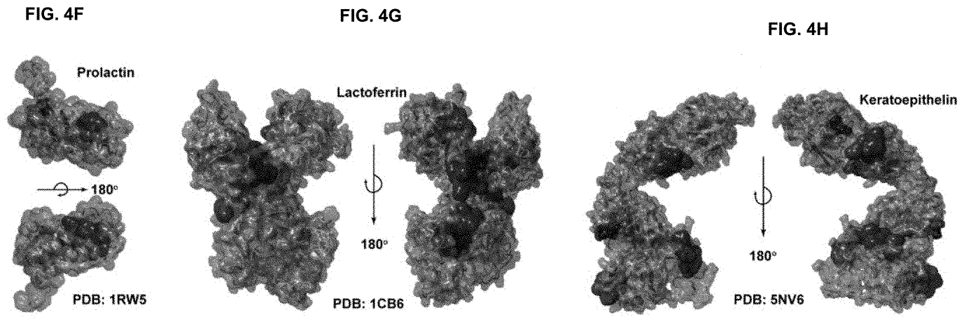

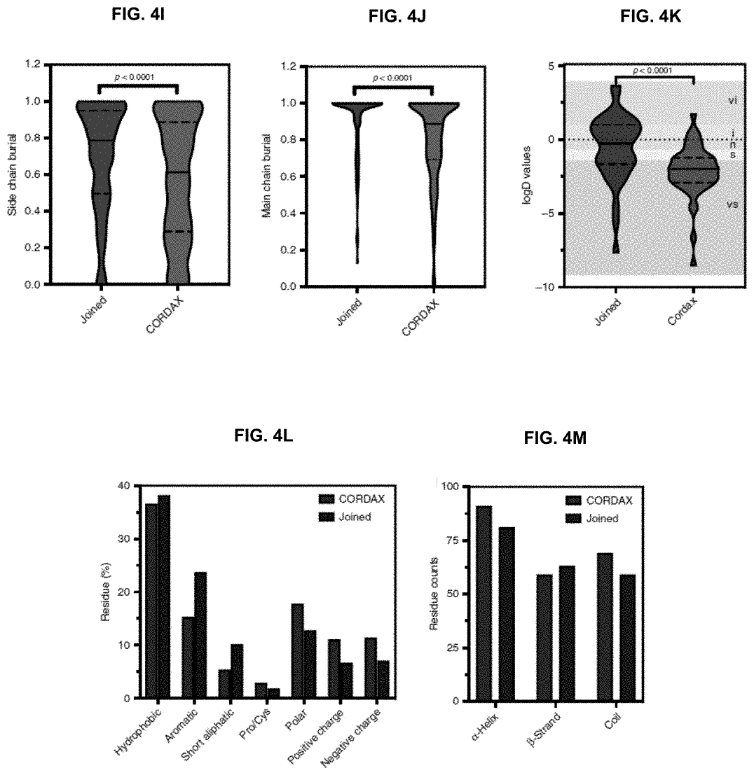

The expanded amyloidogenic annotation of the protein dataset was supplemented with structural analysis of the newly identified aggregation prone regions. Out of 96 peptides designed and experimentally tested, 85 peptides were found to display evident amyloid-forming features, with more than half (55.3%) being predicted specifically by Cordax, contrary to shared predictions with sequence-based tools of high specificity (44.7%) (See Table 2). Pinpointing the location of the identified nucleators in parental protein folds (FIG. 4a) revealed that APRs picked up both by Cordax and traditional sequence-based methods are usually found buried within the core of soluble proteins. Contrary to what has been previously reported^{14,15}, however, our regression model also discovered additional nucleating sequences that primarily appear to reside on the surface of protein molecules (FIG. 4b-h) and as a result, are characterised by high solvent exposure (FIGS. 4i & 4j). Partition coefficients clearly indicate that these exposed peptide segments identified by Cordax are primarily water-soluble sequences, whereas APRs that are predicted by the majority of sequence-based predictors are largely insoluble (FIG. 4k). Sequence distribution analysis signifies that this increased exposure and solubility is complemented by an expected decrease in sequence hydrophobicity (FIG. 4l). More specifically, APRs identified solely by Cordax are relatively enriched in charged or polar side chains (FIG. 4l) and are frequently parts of α-helical or unstructured segments (FIG. 4m). This implies that these regions are in fact conformational switches that may, under fitting misfolding conditions, transiently move towards the formation of β-aggregates. The fact that these sequences are not dictated by typical sequence propensities, such as hydrophobicity or β-structure tendency, explains why sequence-based predictors overlook them.

7. Dimensionality Reduction Transformation Reveals that Cordax Infiltrates Uncharted Areas of Amyloid Sequence Space

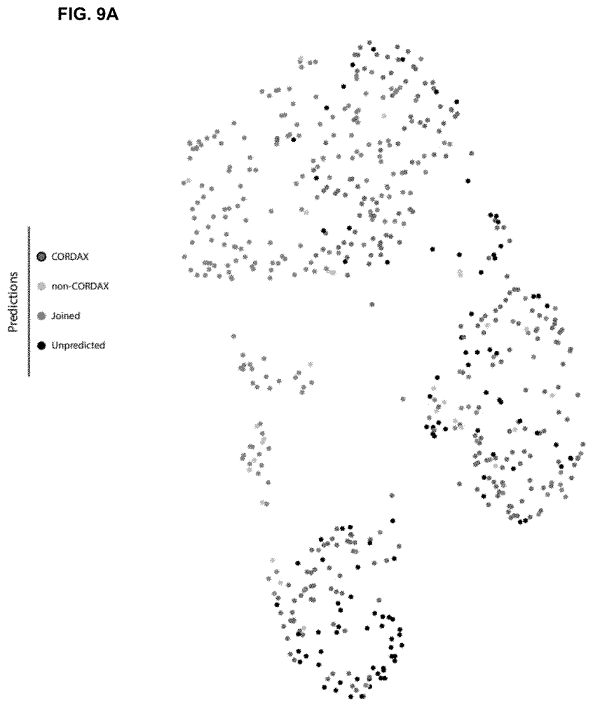

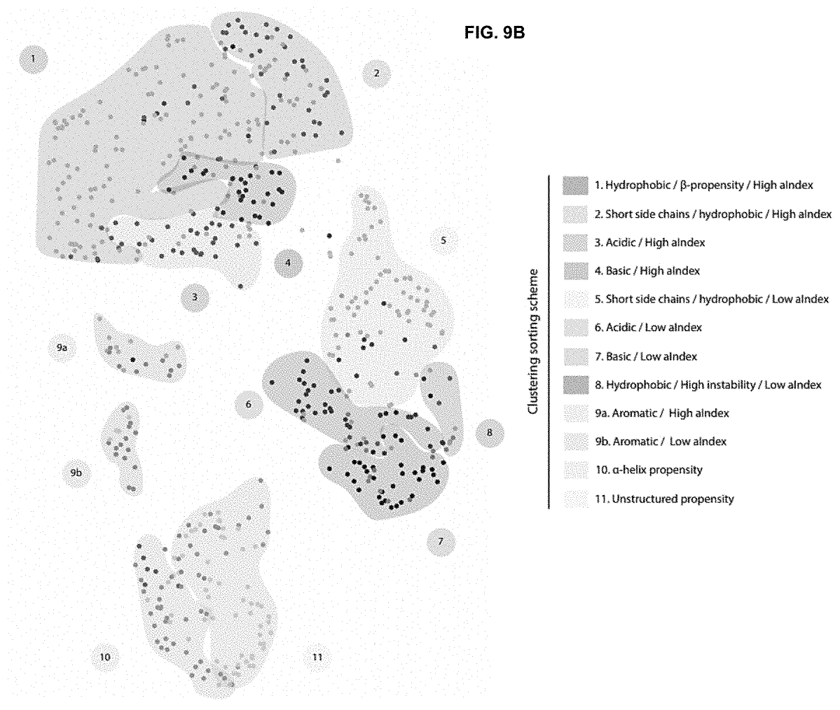

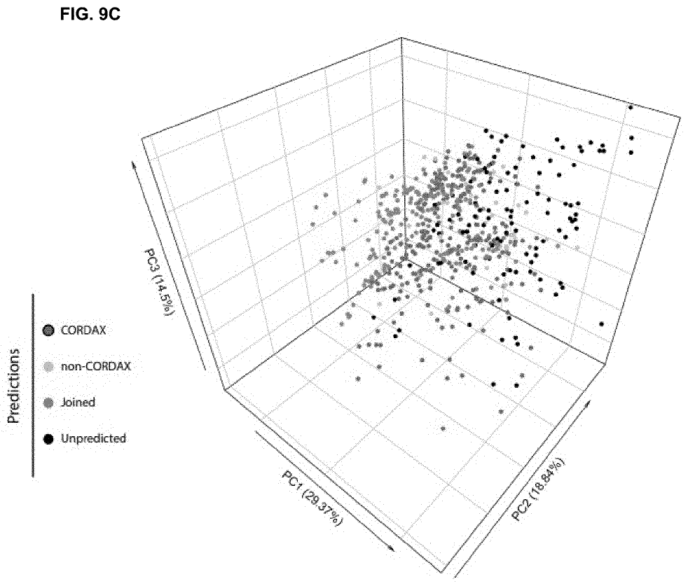

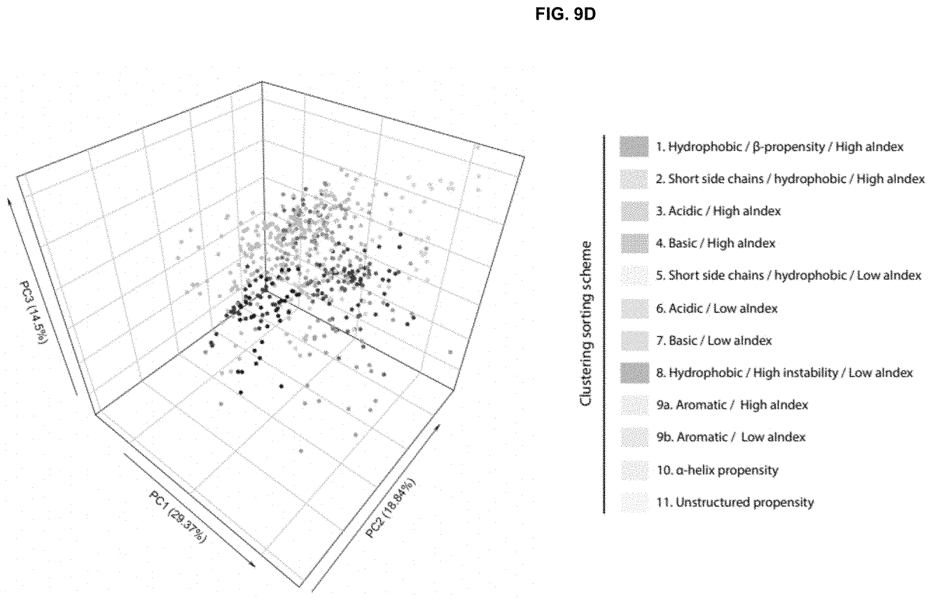

To further explore the capabilities of our method, we composed a map of the known amyloid forming sequence space using t-distributed Stochastic Neighbour Embedding (t-SNE) for dimensionality reduction (FIG. 5a). As input, we used a 20-dimensional parameterisation vector describing all newly identified amyloidogenic peptides merged to the known amyloid-forming hexapeptide sequences in WALTZ-DB, in terms of their basic physicochemical properties and amino acid composition, as well as prediction outputs derived from Cordax and other high specificity predictors. t-SNE mapping pinpointed clear areas of sequence space where Cordax correctly identifies amyloid propensity (purple color in FIG. 5a), which primarily extend towards regions that remain unpredicted (shown in black) and seclude from a large base of sequences identified by multiple methods, including Cordax (cyan colour). Clustering analysis (FIG. 5b) performed using physicochemical properties (FIG. 5c-5e), secondary structure propensities (FIG. 5f) and side chain size distributions (FIG. 5g-h) identifies that this common base of by-now easy to predict APRs are characterised by high hydrophobicity, strong β-sheet propensity and a high relative content of aliphatic side chains (cluster 1 in FIG. 5b), still echoing the initial discovery of APRs by these features⁶. Cordax explores regions adjacent to this with a higher content of shorter side chains (clusters 2 & 5). Notably, amyloid nucleators of this composition are an invaluable resource for amyloid nanomaterial designs with elastin-like properties, are enriched in functional amyloids and have also been linked to ancestral amyloid scaffolds in early life⁴²⁻⁴⁵. A similar trend in amino acid composition has also been reported for proteins that form condensates through phase transition, such as TDP-43 and FUS^{16,18}. Low complexity regions (LCRs) that are enriched in short side chains, such as Gly or Ala, have been shown to drive phase separation, often as an intermediate event towards fibrillation, particularly in polar LCRs with lower aliphatic content and strong disorder or α-helical propensities, such as the sequences discovered in cluster 5^{17,46}. Further to this, Cordax provides significant advancement by traversing in areas with a higher content of negatively or positively charged regions (clusters 3, 4, 6 and 7, respectively). Charged residues often act as gatekeepers that directly disrupt aggregation or modulate it by flanking APRs within protein sequences⁴⁷. Based on this premise, most sequence-based predictors negatively correlate net charge to protein aggregation and have increased failure rates when identifying such amyloid forming stretches. On the other hand, sequences with a high content of aromatic side chains are relatively easy to identify (clusters 9a & 9b), following several lines of evidence supporting their role in amyloid fibril formation⁴⁸. Cordax also pushes forward into less well-charted areas of amyloid sequence space, e.g. exploring clusters with high α-helical content (cluster 10) and overall a low content of aliphatic amino acids (clusters 5, 6, 7, 8 and 9b). These regions also reveal the scope to improve the method, as in particular, the region with high disorder propensity (cluster 11) still contains many false negatives, in spite of the ability of Cordax to partially pick up a minority of sequences. Interestingly, a closer look at the partition coefficients of the known amyloid sequence space reveals that although Cordax takes a significant step forward towards the right direction, these APRs remain very hard to identify as they are characterised by even higher solubility values (FIG. 5i). Similar charting of the amyloid sequence space is achieved by using UMAP (Uniform Manifold Approximation and Projection) for dimensionality reduction (see FIGS. 9a and 9b), while PCA analysis highlights that CORDAX slowly infiltrates the sequence space of higher solubilities (FIGS. 9c and 9d). Overall, dimensionality reduction transformation highlights that structural compatibility can overcome typical sequence propensities as a pivotal driver of aggregation nucleating sequences and suggests that under the proper conditions, the boundaries currently considered compatible to protein amyloid-like assembly are potentially far wider than previously expected.

8. The Cordax Algorithm Predicts the Structural Layout and Overall Topology of Amyloid Fibril Cores

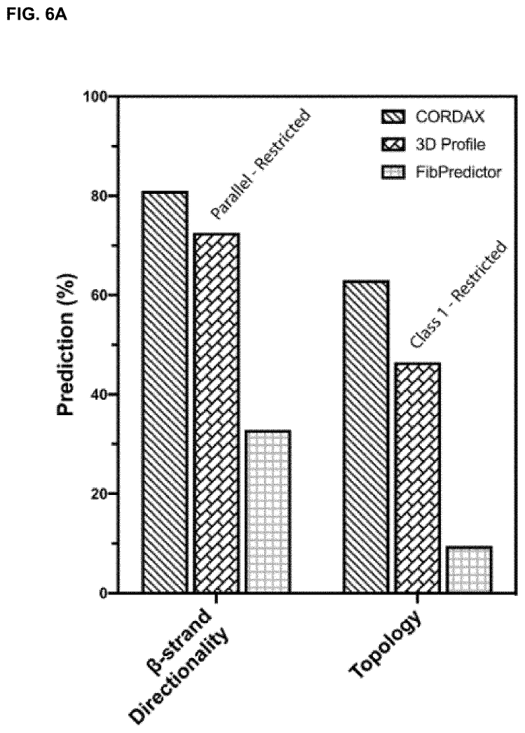

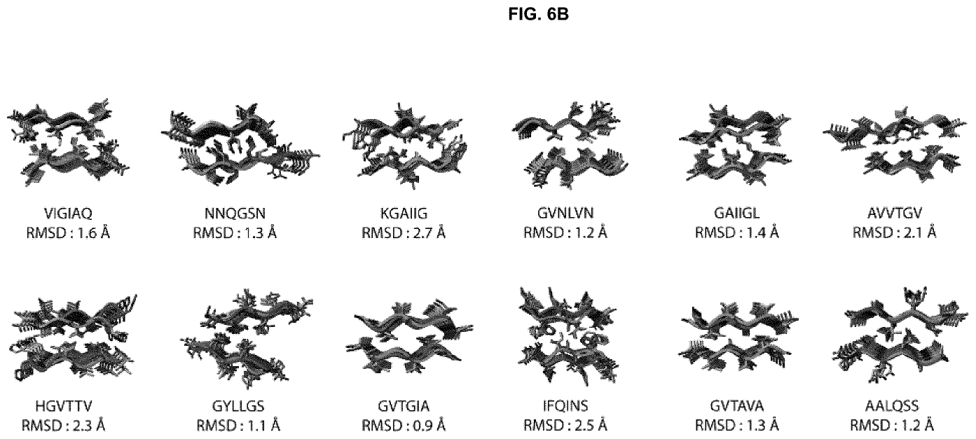

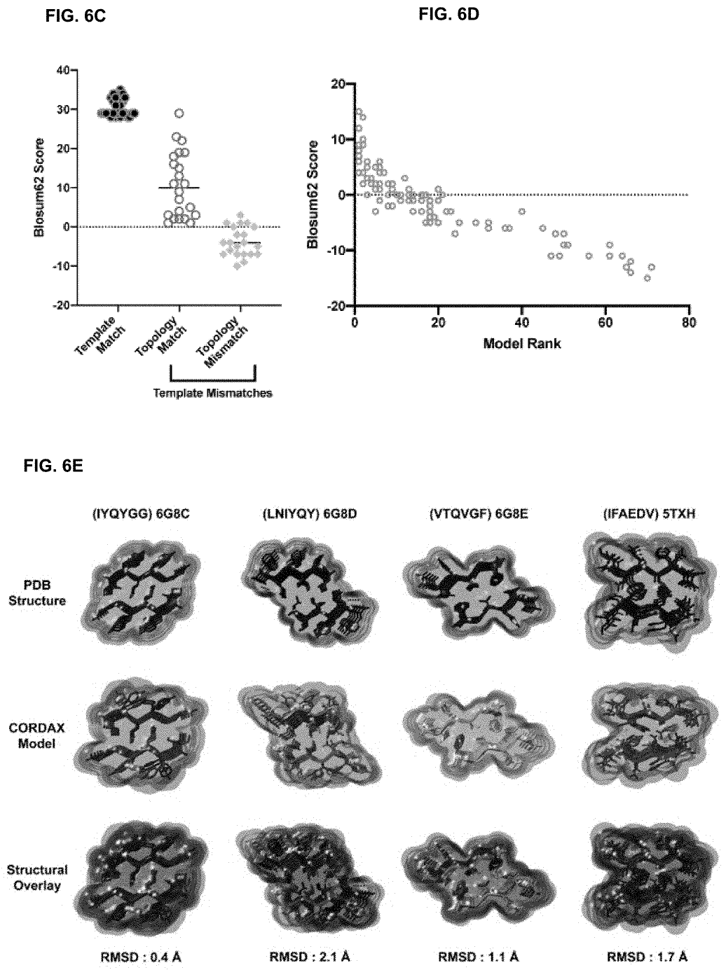

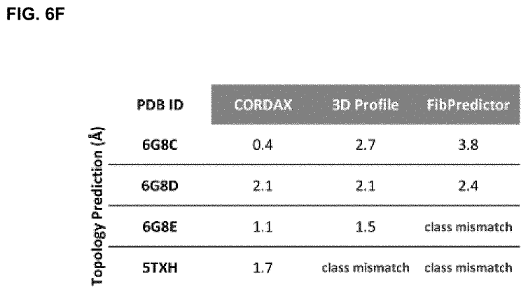

Due to restricted availability of experimentally determined structures not included in the Cordax library, we first analysed the information derived from cross-threading analysis in order to test the performance of the tool in predicting the structural architecture of aggregation prone stretches. Among 73 unique sequences corresponding to the structural library, Cordax was able to accurately assign the correct architecture to 63%, whereas 81% was identified with proper β-strand orientation (parallel/antiparallel) (FIG. 6a, Tables 3 and 5). In comparison, FibPredictor⁴⁹ correct topology allocation was limited to 9.5% of the sequences and assigned β-strand directionality amounted to 32.9%, while introducing an evident preference towards antiparallel architectures (FIG. 6a, Tables 3 and 5). Similarly, the 3D-profile method is restricted to linking all potential queries with a class 1 topology, hence was incapable of predicting alternative architectures (FIG. 6a). Structural alignment indicated that even in cases of mismatching selected templates, modelled architectures strongly superimpose to the solved structures (FIG. 6b), suggesting that Cordax identifies the correct topology with high accuracy. A closer look reveals that sequence specificity may be a modulating, yet not determining factor for this selection process. Steric perturbations can be introduced due to restrictions deriving from closely interdigitating side chains within the packed interfaces, therefore, key residue positions can be bound to the overall stability of certain structural topologies and decrease the acceptable sequence space that can accommodate energetically favourable interactions. This is highlighted by the sequence similarity observed between topological matches (FIG. 6c). On the other hand, topologically different model selections could also be a consequential outcome of amyloid polymorphism. The observed sequence redundancy of the Cordax library illustrates that APRs can form amyloid fibrils with distinct morphological layouts⁵⁰⁻⁵², a notion that is also supported by the common morphological variability of aggregates formed at the level of full-length amyloid-forming proteins^{53,54}. The modulating role of sequence dependency was also evident for the 96-peptide screen. A ranked analysis of the output models indicated that templates with higher alignment scores were not crucial for the topology selection process, although could often correspond to the favourable architectures (FIG. 6d), thus highlighting that the structural predictions of Cordax are relatively unbiased in terms of the sequence space composing the structural templates. The accuracy of the tool was also cross-referenced against experimentally determined structures of fibril cores not included in the structural library. We utilised the recently solved structures of parallel fibril-forming segments derived from the major curli protein CsgA⁵⁵, as well as an anti-parallel polymorphic APR variant segment derived from the amyloid-β peptide⁵⁶. Compared to other structural predictors, only Cordax could invariantly predict the correct architecture for every steric zipper as the closest representation of the experimentally determined reference structures (FIGS. 6e & 6f). This performance can only improve as the fragment library expands, so we aim to update it at regular intervals, providing there is a noticeable increase in solved structures in the future.

9. Cordax Pipeline—Summary

The Cordax algorithm receives a protein sequence in FASTA format as input, which is fragmented into hexapeptides using a sliding window process. Sequences are then threaded against the fragment library utilising FoldX and the derived free energies are translated into scoring values for every peptide window. An energetically fitted model is selected as the closest representative of the overall topology of the amyloid fibril core for each predicted window and is provided as output in standard PDB format to the users (FIG. 10. An amyloidogenic profile is generated by scoring every single residue of the input sequence with the maximum calculated score of the corresponding windows, followed by a binary prediction for every segment. Finally, calculated energies are stored automatically in a growing local database and can be retrieved, thus creating a ‘lazy’ interface that bypasses unnecessary computation for recurring sequence segments or future runs.

10. Datasets

Performance assessment of Cordax was carried out utilising two individual data sets for peptide and protein aggregation propensity detection. Further validation of the method was performed against an independent subset screen of 96 hexapeptides sequences.

WALTZ-DB 2.0 dataset: For peptide aggregation propensity, we used a dataset of 1402 non-redundant hexapeptides contained in the WALTZ-DB 2.0 repository³². This database is the largest currently available resource of experimentally characterized amyloidogenic peptides. It contains annotated peptide entries that are distributed in shorter subsets and extracted from literature^{22,23,67-69}, in addition to peptides with experimentally determined amyloid-forming properties. As a result, it has been widely used as a validation set for several aggregation predicting tools^{21,23,67,70,71}.

Reg33 dataset: Collected in 2013, this is currently a standard dataset for estimating the performance of aggregation propensity prediction in protein sequences²⁵. It contains regional annotation of aggregating segments identified for 34 well-known amyloidogenic proteins. The annotation is assigned on a residue basis, thus containing 1260 residues in defined aggregation prone regions and 6472 residues located in non-aggregating segments.

Cordax validation dataset: This set consists of 96 hexapeptide segments derived from potentially mis-annotated non-amyloidogenic regions of the reg33 dataset that were predicted as aggregation prone segments after applying Cordax. Peptide segments were filtered for potential overlaps to the WALTZ-DB 2.0 set.

11. Comparative Analysis

Binary classification was utilized to determine performances of calculated aggregation propensities per hexapeptide fragment or per residue. As a result, predictions can be classified by comparison to experimental validation into true positives (TP), true negatives (TN), false positives (FP) and false negatives (FN), respectively. Performance is evaluated using the following metrics:

$$Accuracy = \frac{TP + TN}{TP + TN + FP + FN} \\ Precision = \frac{TP}{TP + FP} \\ Sensitivity ( Recall ) = \frac{TP}{TP + FN} \\ Specificity = \frac{TN}{TN + FP} \\ F 1 = 2 \times \frac{( Precision \times Recall )}{( Precision + Recall )} \\ MCC = \frac{( TP \times TN - FP \times FN )}{\sqrt{( TN + FN ) ( TN + FP ) ( TP + FN ) ( TP + FP )}}$$

12. Design of Variant Peptides of a New Aggregation Prone Region (APR) Identified in Apolipoprotein A-I

12.1 Design of Variant Peptides which can Inhibit Aggregation of ApoA-I

A number of naturally occurring mutations of human apolipoprotein A-I (ApoA-I)—see for a reference to this protein: Frank P G and Marcel Y L (2000) J. Lipid Res. 41(6):853) have been associated with hereditary amyloidosis. Amyloidosis are a large group of heterogeneous diseases characterized by insoluble proteins inducing organ damage. Aggregation prone regions are critical regions for the aggregation of proteins able to form pathological aggregates. The Cordax algorithm of the invention was used to identify previously unknown aggregation prone regions (APRs) in apolipoprotein A-I. We identified the sequence LATVYV (SEQ ID NO: 172) present in the amino acid sequence of ApoA-I (corresponding with the amino acid sequence 38 to 43 in the protein sequence of ApoA-I) as a potential new APR.

Based on SEQ ID NO: 172 we explored the design of capping peptides. A capping peptide is a polypeptide which can inhibit the aggregation of a target protein. The term “capping peptide” is well known in the art. Typically, capping peptides have an amino acid length of between 5 and 10 amino acids and differ by one, two or three different amino acid substitutions of a contiguous aggregation prone region (APR) naturally occurring in a target protein.

In building our method we reasoned that for a candidate peptide to qualify as a capping peptide, it should strongly bind to the axial end of a growing amyloid core but at the same time the peptide should introduce sufficient structural disruption which prohibits further elongation along the fibril axis. The latter is in contrast to a wild type (or normal) elongating/nucleating sequence. The method below is illustrated with variants having one amino acid difference as compared to the sequence of the wild type APR region. Our method to design a capping peptide hinges on the availability of the 3D-structure of the amyloid core of SEQ ID NO: 172, here this 3-D structure was modelled based on the Cordax algorithm. Starting from the predicted 3-D structure of the amyloid core structure, a forcefield algorithm was used to calculate the interaction energies between a list of candidate capping peptides (see further) and the 3-D amyloid core structure. In the present example we have used the FoldX force field to calculate the thermodynamic stability of the putative interactions.

The first step in the methodology starts by generating an in silico list of variants of the amino acid sequence of the amyloid core (SEQ ID NO: 172). Thus, starting from the APR sequence an in silico list of variants is created wherein each amino acid in this APR sequence is substituted into all possible 19 different amino acids. In a subsequent step the candidate peptides (consisting of the in silico list of APR variants) are further used for calculating the interaction energies. By plotting the calculated interaction potential calculated through (1) on the x-axis and the potential from (2) on y-axis we end up with a quadratic profile of every of the variant sequences (see FIGS. 10A and 10B). FIGS. 10A and 10B depicts amino acid sequence variants of SEQ ID NO: 172. The top left quadrant corresponds to sequence variants that are predicted to act as potential capping peptides against the identified APR template structure. A favorable variant sequence (in the top left quadrant) has a negative delta G free energy for cross interaction with the three-dimensional structure of the APR core and a positive delta G free energy for elongation with the three-dimensional structure of the APR core with a variant sequence bound to the axial end.

Thus the instant invention provides a method to obtain a set of candidate capping peptides binding to a target protein that forms pathological aggregates comprising the following steps:
a. identifying an APR structure in a target protein,
b. predicting the 3-dimensional (3-D) structure of fibrils produced by said aggregation prone region (APR) amino acid sequence isolated from a target protein,
c. generating an in silico list of variants of said APR amino acid sequence wherein each variant has 1 amino acid difference as compared to the natural APR amino acid sequence,
d. calculating with a Forcefield algorithm the thermodynamic stability for every variant sequence for the interactions between i) the variant sequence and the predicted 3-D structure of the fibrils produced by the APR sequence, this value is designated as the delta Gibbs energy of cross-interaction and ii) the variant sequence and the predicted 3-D structure of fibrils produced by the APR sequence with a variant sequence interacting at its axial end, this value is designated as the delta Gibbs energy of elongation,
e. obtaining at set of candidate capping peptides wherein candidates have a negative delta G free energy for cross-interaction and a positive delta G free energy for elongation, and
f. testing the set of candidate capping peptides and producing one or more capping peptides.

Candidate capping peptide sequences are depicted in Table 6.

TABLE 6
sequences of the capping peptides based on the
APR sequence SEQ ID NO: 172 identified in ApoA-1,
the variant amino acid compared to the wild-type
APR sequence is underlined

| Capping peptide | Sequence Identifier |
|---|---|
| LATKYV | SEQ ID NO: 173 |
| LRTVYV | SEQ ID NO: 174 |
| LWTVYV | SEQ ID NO: 175 |
| LYTVYV | SEQ ID NO: 176 |
| LFTVYV | SEQ ID NO: 177 |

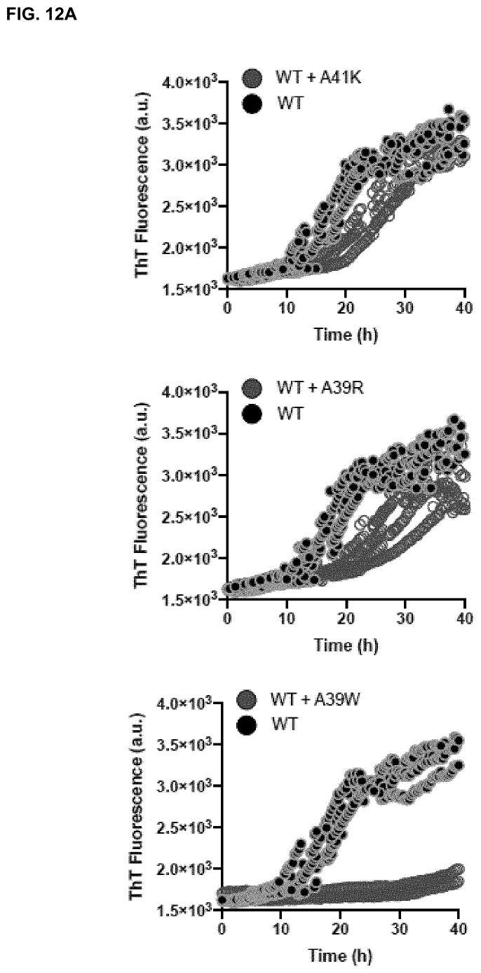

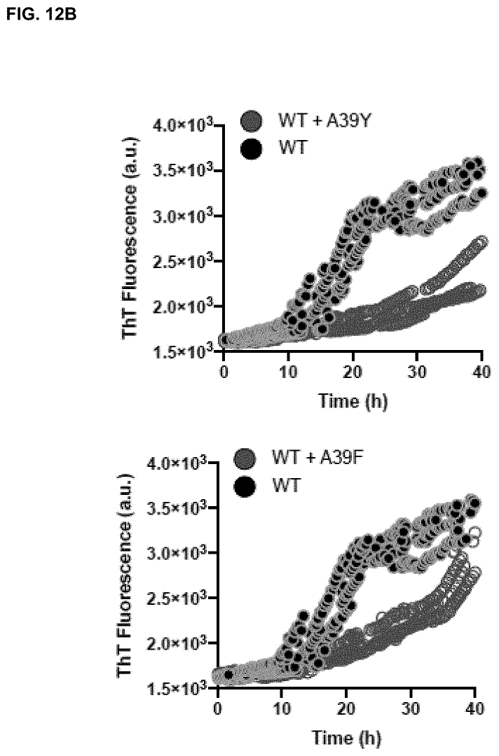

The Th-T kinetics (see FIGS. 12A and 12B) and the endpoint fluorescence analysis (see FIG. 11) were performed in triplicate for the peptides depicted in Table 5. The data confirm that SEQ ID NO: 175 and SEQ ID NO: 176 qualify as most performant capping peptides which can prevent the aggregation of Apo-AI.

12.2 Variant Peptides to Induce Aggregation

In what we can specify as the inverse experiment we also designed peptides which can induce the aggregation of ApoA-I). Here a favorable variant sequence (in the bottom left quadrant) has a negative delta G free energy for cross interaction with the three-dimensional structure of the APR core and also has a negative delta G free energy for elongation with the three-dimensional structure of the APR core with a variant sequence bound to the axial end. The bottom left quadrant corresponds to sequence variants that are predicted to act as aggregation inducing peptides against the identified APR template structure. Table 7 depicts sequences of candidate peptides which can induce the aggregation of Apo-AI.

TABLE 7
sequences of the aggregation inducing peptides
based on the APR sequence SEQ ID NO: 172 identi-
fied in ApoA-1, the variant amino acid compared
to the wild-type APR sequence is underlined.

| Aggregation inducers | Sequence Identifier |
|---|---|
| LLTVYV | SEQ ID NO: 178 |
| LALVYV | SEQ ID NO: 179 |
| LATLYV | SEQ ID NO: 180 |
| LATVYM | SEQ ID NO: 181 |
| LAIVYV | SEQ ID NO: 182 |

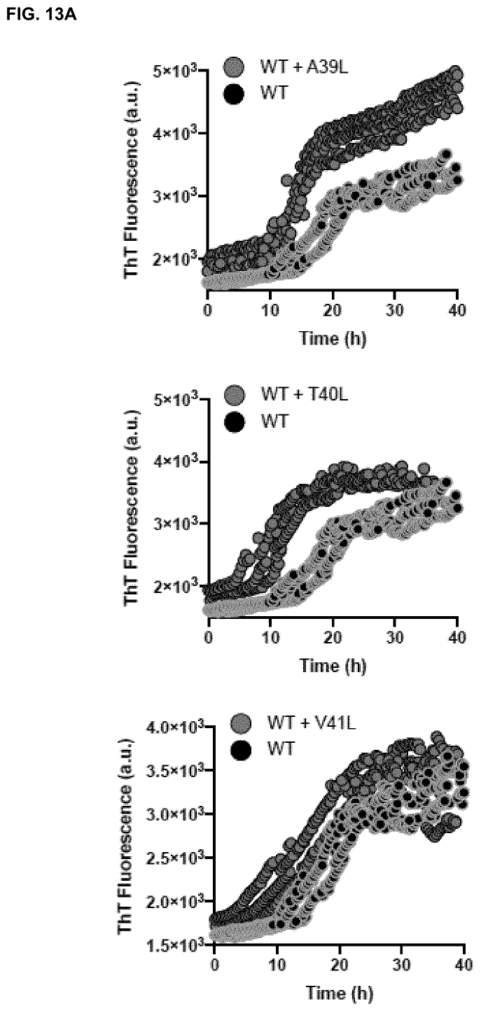

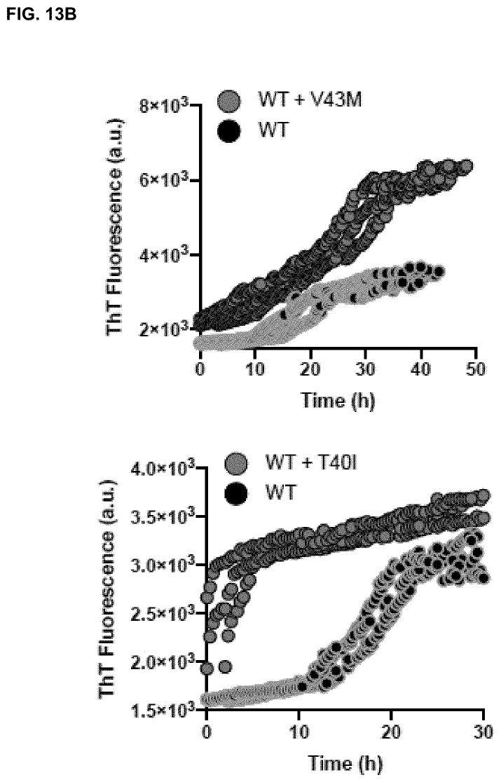

The Th-T kinetics (see FIGS. 13A and 13B) and the endpoint fluorescence analysis (see FIG. 11) were performed in triplicate for the peptides depicted in Table 6. The data confirm that SEQ ID NO: 178 and SEQ ID NO: 182 qualify as most performant peptides which can induce the aggregation of Apo-AI.

Materials and Methods

Peptide Synthesis

Peptides derived from the Cordax validation set were synthesized using an Intavis Multipep RSi solid phase peptide synthesis robot. Peptide purity (>90%) was evaluated using RP-HPLC purification protocols and peptides were stored as ether precipitates (−20° C.). Peptide stocks were initially treated with 1,1,1,3,3,3-hexafluoro-isopropanol (HFIP) (Merck), then dissolved in traces of dimethyl sulfoxide (DMSO) (Merck) (<5%), filtered through 0.2 μm filters and finally in milli-Q water to reach a final concentration of 200 μM or up to 1 mM for dye-negative peptides. Dithiothreitol (DTT) (1 mM) was included in solutions of peptides spanning cysteine or methionine residues. All peptides were incubated at room temperature for a period of 5 days on a rotating wheel.

Thioflavin-T and pFTAA Binding Assays

Amyloid aggregation was monitored using fluorescent spectroscopy binding assays. Th-T (Sigma) or pFTAA (Ebba Biotech AB) was added in half-area black 96-well microplates (Corning, USA) at a final concentration of 25 μM and 0.5 μM, respectively. Fluorescence intensity was measured in replicates (n=6) using a PolarStar Optima and a FluoStar Omega plate reader (BMG Labtech, Germany), equipped with an excitation filter at 440 nm and emission filters at 480 nm and 510 nm, respectively.

Transmission Electron Microscopy

Peptide solutions were incubated for 5 days at room temperature in order to form mature amyloid-like fibrils. Suspensions (5 μL) of each peptide solution were added on 400-mesh carbon-coated copper grids (Agar Scientific Ltd., England), following a glow-discharging step of 30 s to improve sample adsorption. Grids were washed with milli-Q water and negatively stained using uranyl acetate (2% w/v in milli-Q water). Grids were examined with a JEM-1400 120 kV transmission electron microscope (JEOL, Japan), operated at 80 keV.

Congo Red Staining

Droplets (10 μL) of peptide solutions containing mature amyloid fibrils were cast on glass slides and permitted to dry slowly in ambient conditions in order to form thin films. The films were stained with a Congo red (Sigma) solution (0.1% w/v) prepared in milli-Q water for 20 minutes. De-staining was performed with gradient ethanol solutions (70% to 90%).

Determination of Peptide Propensities

Surface exposure and secondary structure analysis was performed using the FoldX energy force field on the available crystal structures for acylphosphatase-2 (PDB ID:1APS), amphoterin (PDB ID:1CKT and 1HME), apolipoprotein-C2 (PDB ID:115J), α-synuclein (PDB ID:1XQ8), β2-microglobulin (PDB ID:1A1M), casein (PDB ID:6FS5), gelsolin (PDB ID:3FFN), Het-S (PDB ID:2WVN), kerato-epithelin (PDB ID:5NV6), lactoferrin (PDB ID:1CB6), prolactin (PDB ID:1RW5), major prion protein (PDB ID:1E1G), repA (PDB ID:1HKQ), serum amyloid alpha (PDB ID:41P8), Sup35 (PDB ID:4CRN) and Ure2p (PDB ID:1HQO). Partition coefficients were calculated using P log P, which specialises in peptides with blocked termini⁷². Structural alignment and visualisation were performed with the aid of YASARA⁷³. Sequence similarities were calculated using the BLOSUM62 matrix currently available under the Biostrings R library. Correlation plots were generated using the ggpairs( ) function available under the GGally R library and ROC curves were calculated using ROCR.

Dimensionality Reduction Analysis

A defined amyloid-forming sequence space was constructed by merging the experimentally determined amyloid sequences of the 96-peptide screen, identified by Cordax, to the amyloid sequence content extracted from WALTZ-DB. Prior to t-SNE analysis, scoring outputs using Cordax, PASTA²³, TANGO⁷ and WALTZ²¹ were calculated for each peptide entry. Peptide description was complemented with a 20-dimensional vector using the available R package Peptides. All data points were reduced and embedded in 2D-space using the Rtsne package, with perplexity (p=45), iteration steps (n=5000) and learning rate (default) defined based on the initial guidelines proposed by van der Maaten & Hinton⁷⁴. UMAP reduction was performed using the R umap package and three-dimensional PCA analysis was conducted using pca3d R package and visualised with scatter3D, respectively.

Tables 1 to 5

TABLE 1
List of templates incorporated in individual processing
steps during generation of the CORDAX structural library.

| | Interface/Length | Energy | Promiscuity | CORDAX |
|---|---|---|---|---|
| PDB ID | Fragments | Refinement | Sorting | Library |
| 78 | 179 | −33 | −6 | 140 |
| structures | templates | templates | templates | templates |
| 1yjo | 1yjo | | 1yjo | |
| 1yjp | 1yjp_1 | | | 1yjp_1 |
| | 1yjp_2 | | | 1yjp_2 |
| 2kib | 2kib_1 | | | 2kib_1 |
| | 2kib_2 | | | 2kib_2 |
| 2m5n | 2m5n_1 | 2m5n_1 | | |
| | 2m5n_2 | | | 2m5n_2 |
| | 2m5n_3 | | | 2m5n_3 |
| | 2m5n_4 | | | 2m5n_4 |
| | 2m5n_5 | 2m5n_5 | | |
| | 2m5n_6 | 2m5n_6 | | |
| 2okz | 2okz_a | | | 2okz_a |
| 2ol9 | 2ol9 | | | 2ol9 |
| 2omm | 2omm_1 | | | 2omm_1 |
| | 2omm_2 | | | 2omm_2 |
| 2omp | 2omp | | | 2omp |
| 2omq | 2omq | | | 2omq |
| 2on9 | 2on9 | | | 2on9 |
| 2ona | 2ona | | | 2ona |
| 2onv | 2onv_a | | | 2onv_a |
| 2onw | 2onw | | | 2onw |
| 2y29 | 2y29 | | | 2y29 |
| 2y2a | 2y2a | | | 2y2a |
| 2y3j | 2y3j | | | 2y3j |
| 2y3k | 2y3k_1 | | | 2y3k_1 |
| | 2y3k_2 | | | 2y3k_2 |
| | 2y3k_3 | | | 2y3k_3 |
| 2y3l | 2y3l_1 | | | 2y3l_1 |
| | 2y3l_2 | | | 2y3l_2 |
| | 2y3l_3 | | | 2y3l_3 |
| 3dg1 | 3dg1_a | | | 3dg1_a |
| | 3dg1_b | | | 3dg1_b |
| 3dgj | 3dgj_1 | | | 3dgj_1 |
| | 3dgj_2 | | | 3dgj_2 |
| 3fod | 3fod | | 3fod | |
| 3fpo | 3fpo | | | 3fpo |
| 3fr1 | 3fr1 | | 3fr1 | |
| 3fth | 3fth_1 | | | 3fth_1 |
| | 3fth_2 | | | 3fth_2 |
| 3ftk | 3ftk_1 | | | 3ftk_1 |
| | 3ftk_2 | | | 3ftk_2 |
| 3ftl | 3ftl_a_1 | | | 3ftl_a_1 |
| | 3ftl_a_2 | | | 3ftl_a_2 |
| | 3ftl_b_1 | | | 3ftl_b_1 |
| | 3ftl_b_2 | | | 3ftl_b_2 |
| 3ftr | 3ftr_a | | | 3ftr_a |
| | 3ftr_b | | | 3ftr_b |
| 3fva | 3fva_a | | | 3fva_a |
| | 3fva_b | | | 3fva_b |
| 3hyd | 3hyd_a_1 | | | 3hyd_a_1 |
| | 3hyd_a_2 | | | 3hyd_a_2 |
| | 3hyd_b_1 | | | 3hyd_b_1 |
| | 3hyd_b_2 | | | 3hyd_b_2 |
| 3loz | 3loz | | | 3loz |
| 3nhc | 3nhc | | | 3nhc |
| 3nhd | 3nhd | | | 3nhd |
| 3nve | 3nve | | | 3nve |
| 3ow9 | 3ow9 | | | 3ow9 |
| 3ppd | 3ppd_a | | | 3ppd_a |
| | 3ppd_b | | | 3ppd_b |
| 3pzz | 3pzz | | | 3pzz |
| 3q2x | 3q2x_a | | | 3q2x_a |
| | 3q2x_b | | | 3q2x_b |
| 3sgs | 3sgs | | | 3sgs |
| 4nin | 4nin_1 | | | 4nin_1 |
| | 4nin_2 | | | 4nin_2 |
| 4nio | 4nio_a_1 | | | 4nio_a_1 |
| | 4nio_a_2 | | | 4nio_a_2 |
| | 4nio_b_2 | | | 4nio_b_2 |
| 4nip | 4nip_a_1 | | | 4nip_a_1 |
| | 4nip_a_2 | | | 4nip_a_2 |
| | 4nip_b_1 | | | 4nip_b_1 |
| | 4nip_b_2 | | | 4nip_b_2 |
| 4np8 | 4np8 | | | 4np8 |
| 4r0p | 4r0p_a | | | 4r0p_a |
| | 4r0p_b | | | 4r0p_b |
| 4r0u | 4r0u_a_1 | | | 4r0u_a_1 |
| | 4r0u_a_2 | | | 4r0u_a_2 |
| | 4r0u_b_1 | | | 4r0u_b_1 |
| | 4r0u_b_2 | | | 4r0u_b_2 |
| 4r0w | 4r0w_a_1 | | | 4r0w_a_1 |
| | 4r0w_a_2 | | | 4r0w_a_2 |
| | 4r0w_b_1 | 4r0w_b_1 | | |
| | 4r0w_b_2 | | | 4r0w_b_2 |
| 4rik | 4rik_a_1 | 4rik_a_1 | | |
| | 4rik_a_2 | | | 4rik_a_2 |
| | 4rik_a_3 | | | 4rik_a_3 |
| | 4rik_a_4 | | | 4rik_a_4 |
| | 4rik_b_1 | | | 4rik_b_1 |
| | 4rik_b_2 | | | 4rik_b_2 |
| | 4rik_b_3 | | | 4rik_b_3 |
| | 4rik_b_4 | 4rik_b_4 | | |
| 4ril | 4ril_a_1 | 4ril_a_1 | | |
| | 4ril_a_2 | | | 4ril_a_2 |
| | 4ril_a_3 | | | 4ril_a_3 |
| | 4ril_a_4 | | | 4ril_a_4 |
| | 4ril_a_5 | 4ril_a_5 | | |
| | 4ril_a_6 | 4ril_a_6 | | |
| | 4ril_b_1 | 4ril_b_1 | | |
| | 4ril_b_2 | 4ril_b_2 | | |
| | 4ril_b_3 | | | 4ril_b_3 |
| | 4ril_b_4 | | | 4ril_b_4 |
| | 4ril_b_5 | | | 4ril_b_5 |
| | 4ril_b_6 | 4ril_b_6 | | |
| 4rp6 | 4rp6_1 | | | 4rp6_1 |
| | 4rp6_2 | | | 4rp6_2 |
| 4rp7 | 4rp7_a | | | 4rp7_a |
| | 4rp7_b | | | 4rp7_b |
| 4tut | 4tut | | | 4tut |
| 4uby | 4uby | | | 4uby |
| 4ubz | 4ubz | | | 4ubz |
| 4w5l | 4w5l_1 | | | 4w5l_1 |
| | 4w5l_2 | | | 4w5l_2 |
| 4w5m | 4w5m_1 | | | 4w5m_1 |
| | 4w5m_2 | | | 4w5m_2 |
| 4w5p | 4w5p_1 | | | 4w5p_1 |
| | 4w5p_2 | | | 4w5p_2 |
| 4w5y | 4w5y_1 | | | 4w5y_1 |
| | 4w5y_2 | 4w5y_2 | | |
| 4w67 | 4w67_1 | | | 4w67_1 |
| | 4w67_2 | | 4w67_2 | |
| 4w71 | 4w71_1 | | | 4w71_1 |
| | 4w71_2 | | | 4w71_2 |
| 4wbu | 4wbu | | | 4wbu |
| 4wbv | 4wbv | | | 4wbv |
| 4xfn | 4xfn | | 4xfn | |
| 4xfo | 4xfo | | | 4xfo |
| 4znn | 4znn_a_1 | 4znn_a_1 | | |
| | 4znn_a_2 | 4znn_a_2 | | |
| | 4znn_a_3 | | | 4znn_a_3 |
| | 4znn_a_4 | | | 4znn_a_4 |
| | 4znn_a_5 | | | 4znn_a_5 |
| | 4znn_b_1 | 4znn_b_1 | | |
| | 4znn_b_2 | 4znn_b_2 | | |
| | 4znn_b_3 | | | 4znn_b_3 |
| | 4znn_b_4 | | | 4znn_b_4 |
| | 4znn_b_5 | | | 4znn_b_5 |
| 5e5c | 5e5c | | | 5e5c |
| 5e5v | 5e5v_1 | | | 5e5v_1 |
| | 5e5v_2 | | | 5e5v_2 |
| 5e5x | 5e5x | | | 5e5x |
| 5e5z | 5e5z | | | 5e5z |
| 5n9i | 5n9i | | | 5n9i |
| 5vos | 5vos_a_1 | 5vos_a_1 | | |
| | 5vos_a_2 | 5vos_a_2 | | |
| | 5vos_a_3 | | | 5vos_a_3 |
| | 5vos_a_4 | | | 5vos_a_4 |
| | 5vos_a_5 | | | 5vos_a_5 |
| | 5vos_a_6 | 5vos_a_6 | | |
| | 5vos_b_1 | 5vos_b_1 | | |
| | 5vos_b_2 | 5vos_b_2 | | |
| | 5vos_b_3 | 5vos_b_3 | | |
| | 5vos_b_4 | 5vos_b_4 | | |
| | 5vos_b_5 | | | 5vos_b_5 |
| | 5vos_b_6 | | | 5vos_b_6 |
| 5w52 | 5w52_1 | 5w52_1 | | |
| | 5w52_2 | 5w52_2 | | |
| | 5w52_3 | 5w52_3 | | |
| | 5w52_4 | 5w52_4 | | |
| | 5w52_5 | | | 5w52_5 |
| | 5w52_6 | 5w52_6 | | |
| 5whp | 5whp | | | 5whp |
| 5wia | 5wia | | | 5wia |
| 5wiq | 5wiq_1 | | | 5wiq_1 |
| | 5wiq_2 | | | 5wiq_2 |
| 5wkb | 5wkb | | | 5wkb |
| 5wkd | 5wkd_a_1 | | | 5wkd_a_1 |
| | 5wkd_a_2 | | | 5wkd_a_2 |
| | 5wkd_b_1 | | | 5wkd_b_1 |
| | 5wkd_b_2 | | | 5wkd_b_2 |
| 6cb9 | 6cb9_a | | | 6cb9_a |
| | 6cb9_b | | | 6cb9_b |
| 6cew | 6cew | | | 6cew |
| 6cfh | 6cfh_1 | 6cfh_1 | | |
| | 6cfh_2 | 6cfh_2 | | |
| | 6cfh_3 | | 6cfh_3 | |
| | 6cfh_4 | | | 6cfh_4 |
| | 6cfh_5 | 6cfh_5 | | |
| | 6cfh_6 | 6cfh_6 | | |

TABLE 2
Amyloidogenic properties of the Cordax-predicted peptide screen.

| | | Predic- | | Cordax | | | | Th-T | pFTAA | | |
|---|---|---|---|---|---|---|---|---|---|---|---|
| Class | Sequence | tion | Protein | Score | PASTA2 | WALTZ | TANGO | Binding | Binding | TEM | Amyloid |
| Amyloid | SVDYEV | CORDAX | Acylphospha- | 0.93 | −0.43295 | 0 | 0.008483933 | — | ● | | ● |
| | | | tase-2 | | | | | | | | |
| Amyloid | IGVVGW | Joined | Acylphospha- | 0.8 | −4.64595 | 9.681 | 0.003878967 | ● | ● | | ● |
| | | | tase-2 | | | | | | | | |
| Amyloid | NFSIRY | CORDAX | Acylphospha- | 0.978 | −2.28288 | 1.6111989 | 0 | ● | ● | ● | ● |
| | | | tase-2 | | | | | | | | |
| Amyloid | VNFSEF | Joined | Amphoterin | 0.82 | −2.20553 | 0 | 27.99087667 | ● | ● | ● | ● |
| Amyloid | SEFSKK | Joined | Amphoterin | 0.86 | 0.0041 | 0 | 8.683348333 | — | ● | | ● |
| Amyloid | AFFLFC | Joined | Amphoterin | 0.93 | −3.51912 | 76.34016667 | 94.74496667 | ● | ● | | ● |
| Non- | LSSYWE | CORDAX | Apolipoprotein | 0.91 | −0.76007 | 0 | 0.03827015 | — | — | — | — |
| Amyloid | | | C2 | | | | | | | | |
| Amyloid | NLYEKT | Joined | Apolipoprotein | 0.9 | −0.6145 | 0 | 56.35599082 | — | ● | | ● |
| | | | C2 | | | | | | | | |
| Amyloid | VYVDVL | Joined | Apolipoprotein | 0.83 | −5.71601 | 0.020776 | 28.097125 | ● | ● | ● | ● |
| | | | A1 | | | | | | | | |
| Amyloid | LNLKLL | CORDAX | Apolipoprotein | 0.82 | −1.66577 | 0.000882504 | 0 | — | ● | | ● |
| | | | A1 | | | | | | | | |
| Amyloid | LLDNWD | CORDAX | Apolipoprotein | 0.88 | −0.905443 | 0.00100333 | 0 | — | ● | | ● |
| | | | A1 | | | | | | | | |
| Amyloid | MDVFMK | CORDAX | Alpha-synuclein | 0.98 | −1.76345 | 0 | 0 | ● | ● | | ● |
| Amyloid | IQVYSR | CORDAX | Beta-2- | 0.83 | −2.97686 | 0.0267759 | 0 | ● | ● | ● | ● |
| | | | microglobulin | | | | | | | | |
| Amyloid | LCSTFC | CORDAX | Casein | 0.89 | −3.52309 | 0.190833333 | 0.19959 | ● | ● | | ● |
| Non- | LNFLKK | CORDAX | Casein | 0.98 | −2.38069 | 0 | 0.031414065 | — | — | — | — |
| Amyloid | | | | | | | | | | | |
| Non- | KTVYQH | CORDAX | Casein | 0.91 | −2.20788 | 0 | 0.000562 | — | — | — | — |
| Amyloid | | | | | | | | | | | |
| Amyloid | TKVIPY | CORDAX | Casein | 0.97 | −2.71835 | 0 | 0.004041338 | — | ● | | ● |
| Amyloid | MVVEHP | CORDAX | Gelsolin | 0.97 | −1.63041 | 0 | 0 | — | ● | | ● |
| Amyloid | AYVILK | Joined | Gelsolin | 0.87 | −5.16742 | 64.5065 | 0.142594167 | — | ● | | ● |
| Amyloid | GWFLGW | Joined | Gelsolin | 0.83 | −2.11341 | 51.71333333 | 1.639952401 | ● | — | | ● |
| Amyloid | YDLHYW | CORDAX | Gelsolin | 0.98 | −2.00912 | 0 | 0.654945333 | ● | ● | ● | ● |
| Amyloid | IFTVQL | Joined | Gelsolin | 0.94 | −4.94671 | 39.44816667 | 6.468355083 | ● | ● | ● | ● |
| Amyloid | FLGYFK | Joined | Gelsolin | 0.93 | −1.73416 | 21.89833333 | 0.3201015 | — | — | ● | ● |
| Amyloid | MSVSLV | CORDAX | Gelsolin | 0.88 | −4.09918 | 1.367 | 0 | ● | ● | | ● |
| Amyloid | EDCFIL | CORDAX | Gelsolin | 0.94 | −3.68898 | 0 | 1.875415 | ● | ● | | ● |
| Non- | KIFVWK | Joined | Gelsolin | 0.84 | −5.76718 | 0 | 0.0527492 | — | — | — | — |
| Amyloid | | | | | | | | | | | |
| Non- | DFITKM | CORDAX | Gelsolin | 0.89 | −1.78202 | 0 | 0.01066185 | — | — | — | — |
| Amyloid | | | | | | | | | | | |
| Amyloid | YIILYN | Joined | Gelsolin | 0.98 | −5.56975 | 76.70633333 | 89.9522 | ● | ● | ● | ● |
| Amyloid | IIYNWQ | Joined | Gelsolin | 0.98 | −5.2583 | 7.727333333 | 66.182978 | ● | ● | ● | ● |
| Amyloid | MIIYKG | CORDAX | Gelsolin | 0.98 | −4.55769 | 0 | 0.004878478 | — | ● | | ● |
| Amyloid | NDAFVL | CORDAX | Gelsolin | 0.86 | −3.14957 | 0 | 0.014878623 | ● | ● | ● | ● |
| Amyloid | AAYLWV | Joined | Gelsolin | 0.95 | −4.09608 | 65.3925 | 5.92667 | ● | ● | ● | ● |
| Amyloid | FVIEEV | CORDAX | Gelsolin | 0.89 | −2.24467 | 0 | 0.0422345 | ● | ● | ● | ● |
| Amyloid | QVFVWV | Joined | Gelsolin | 0.8 | −7.803 | 83.21533333 | 0.028962867 | ● | ● | ● | ● |
| Non- | GIVAGA | Joined | Het-S | 0.86 | −2.1412 | 16.43083333 | 0.00031171 | — | — | — | — |
| Amyloid | | | | | | | | | | | |
| Amyloid | DCFEYV | Joined | Het-S | 0.94 | −2.23094 | 0 | 14.4493 | ● | ● | | ● |
| Amyloid | FEYVQL | Joined | Het-S | 0.89 | −1.90226 | 0 | 9.6328 | ● | ● | | ● |
| Amyloid | ILLLFE | Joined | Het-S | 0.9 | −5.13194 | 40.64583333 | 3.440353333 | ● | ● | ● | ● |
| Amyloid | DLVVFE | Joined | Het-S | 0.96 | −5.65432 | 0 | 0.913069667 | ● | ● | | ● |
| Amyloid | AEIEIE | CORDAX | Het-S | 0.87 | −0.58904 | 0 | 0.316384333 | ● | ● | ● | ● |
| Amyloid | ASLTIL | CORDAX | Het-S | 0.85 | −3.56673 | 1.974333333 | 0.774627 | ● | ● | ● | ● |
| Amyloid | YQLVLQ | CORDAX | Keratoepithelin | 0.84 | −2.51775 | 2.530833333 | 0.00144408 | ● | ● | ● | ● |
| Amyloid | STVISY | CORDAX | Keratoepithelin | 0.84 | −3.82553 | 4.678666667 | 0.083860133 | ● | ● | ● | ● |
| Amyloid | VNIELL | CORDAX | Keratoepithelin | 0.95 | −2.84689 | 0.241333333 | 3.150656667 | ● | ● | | ● |
| Amyloid | IQIHHY | CORDAX | Keratoepithelin | 0.97 | −2.9305 | 0 | 0.207938573 | — | — | ● | ● |
| Amyloid | QIIEIE | Joined | Keratoepithelin | 0.95 | −3.29635 | 0 | 41.09278717 | ● | ● | | ● |
| Amyloid | GVIHYI | Joined | Keratoepithelin | 0.87 | −6.30048 | 1.297666667 | 3.939081667 | — | — | ● | ● |
| Amyloid | LNSVFK | CORDAX | Keratoepithelin | 0.91 | −2.96225 | 0.4625 | 0.000978769 | — | — | ● | ● |
| Amyloid | LYHGQT | CORDAX | Keratoepithelin | 0.93 | 0.1047 | 0 | 0.112152947 | — | — | ● | ● |
| Amyloid | TLFTMD | CORDAX | Keratoepithelin | 0.92 | −2.5105 | 4.713333333 | 0.011382033 | — | — | ● | ● |
| Amyloid | VYTVFA | Joined | Keratoepithelin | 0.91 | −5.41231 | 49.937 | 0.016631933 | ● | ● | | ● |
| Amyloid | HYYAVA | Joined | Lactoferrin | 0.87 | −2.37155 | 54.921 | 0.005968873 | — | ● | | ● |
| Amyloid | GDVAFI | Joined | Lactoferrin | 0.92 | −4.3654 | 0 | 39.64781667 | ● | ● | | ● |
| Amyloid | LLFKDS | CORDAX | Lactoferrin | 0.87 | −1.06354 | 0 | 0.377282833 | — | — | ● | ● |
| Amyloid | YFTAIQ | Joined | Lactoferrin | 0.94 | −2.87592 | 13.8145 | 0.420026 | ● | ● | | ● |
| Amyloid | GWNIPM | Joined | Lactoferrin | 0.91 | −1.51682 | 0.381666667 | 7.139811667 | — | ● | | ● |
| Amyloid | MDKVER | CORDAX | Lactoferrin | 0.92 | −1.3496 | 0 | 0 | — | — | ● | ● |
| Amyloid | YVAGIT | CORDAX | Lactoferrin | 0.93 | −2.65591 | 3.3695 | 0.014292333 | ● | ● | | ● |
| Amyloid | CEFLRK | CORDAX | Lactoferrin | 0.9 | −1.62345 | 0 | 0.0244978 | — | ● | | ● |
| Amyloid | LQVDLG | CORDAX | Medin | 0.83 | −0.596554 | 0 | 0 | ● | ● | | ● |
| Non- | FDKFKH | CORDAX | Myoglobin | 0.86 | −0.82392 | 0 | 0.012595033 | — | — | — | — |
| Amyloid | | | (Horse) | | | | | | | | |
| Amyloid | MFSEFD | CORDAX | Prolactin | 0.92 | −0.98176 | 0 | 1.03462 | ● | ● | | ● |
| Amyloid | LIVSIL | Joined | Prolactin | 0.95 | −6.79681 | 85.78183333 | 2.2071945 | ● | ● | | ● |
| Amyloid | LYHLVT | CORDAX | Prolactin | 0.85 | −3.94956 | 0 | 0.015143467 | — | — | ● | ● |
| Amyloid | MELIVS | Joined | Prolactin | 0.93 | −4.06754 | 0.787833333 | 13.64441667 | ● | ● | | ● |
| Amyloid | LHCLRR | CORDAX | Prolactin | 0.93 | −1.50115 | 0.319166667 | 0.024989667 | — | ● | | ● |
| Amyloid | QVYYRP | Joined | Major Prion | 0.91 | −2.4323 | 0 | 79.2739 | — | — | ● | ● |
| | | | Protein (PrP) | | | | | | | | |
| Amyloid | GYLTIR | CORDAX | RepA | 0.88 | −3.24397 | 0.5975 | 0.543358 | — | — | ● | ● |
| Non- | KLFNRD | CORDAX | RepA | 0.95 | −0.37476 | 0 | 0.121829333 | — | — | — | — |
| Amyloid | | | | | | | | | | | |
| Non- | FHVKYR | CORDAX | RepA | 0.99 | −2.11898 | 0.111 | 0.0165435 | — | — | — | — |
| Amyloid | | | | | | | | | | | |
| Amyloid | MLHKEF | CORDAX | RepA | 0.9 | −0.51634 | 0 | 0.002294787 | ● | — | | ● |
| Amyloid | FYAVRL | Joined | RepA | 0.9 | −2.82682 | 6.504 | 1.561631167 | — | — | ● | ● |
| Amyloid | SQFIKL | CORDAX | RepA | 0.95 | −2.27937 | 0.706666667 | 0.035492557 | — | — | ● | ● |
| Amyloid | FSFTIA | Joined | RepA | 0.94 | −3.16584 | 16.80766667 | 3.881723333 | ● | ● | | ● |
| Amyloid | YFHARG | CORDAX | SAA | 0.88 | −1.85073 | 0 | 0.01291105 | — | — | ● | ● |
| Amyloid | LIFMGH | Joined | Sup35 | 0.91 | −3.79713 | 31.89983333 | 13.90792167 | ● | ● | | ● |
| Amyloid | MYVSEM | CORDAX | Sup35 | 0.89 | −1.95087 | 0 | 0.00126159 | ● | ● | | ● |
| Amyloid | VVVNKM | Joined | Sup35 | 0.9 | −5.99058 | 37.031 | 0 | — | ● | | ● |
| Amyloid | SNFLRA | Joined | Sup35 | 0.87 | −0.95806 | 0 | 11.37023649 | — | — | ● | ● |
| Amyloid | IGYNIK | CORDAX | Sup35 | 0.91 | −3.02212 | 0.237666667 | 0.043181233 | — | — | ● | ● |
| Amyloid | TDVVFM | Joined | Sup35 | 0.97 | −5.07623 | 0.8275 | 24.9153 | ● | ● | | ● |
| Amyloid | VDMAMC | CORDAX | Sup35 | 0.89 | −0.44413 | 0 | 0 | ● | ● | | ● |
| Amyloid | VHIVKL | Joined | Sup35 | 0.96 | −5.89815 | 0 | 2.391173467 | — | ● | ● | ● |
| Amyloid | INFEFS | CORDAX | Ure2p | 0.94 | −1.11595 | 0 | 0 | ● | ● | | ● |
| Amyloid | GYTLFS | CORDAX | Ure2p | 0.92 | −2.16066 | 2.631666667 | 1.178483333 | — | — | ● | ● |
| Amyloid | FKVAIV | Joined | Ure2p | 0.91 | −5.48036 | 61.7 | 0.1212853 | — | ● | | ● |
| Amyloid | TIFLDF | Joined | Ure2p | 0.94 | −3.69924 | 2.0915 | 6.519154 | — | ● | | ● |
| Amyloid | LSIWES | CORDAX | Ure2p | 0.93 | −2.96457 | 0 | 0.737657802 | ● | ● | | ● |
| Non- | HLVNKY | CORDAX | Ure2p | 0.94 | −2.65616 | 1.853 | 0.983641913 | — | — | — | — |
| Amyloid | | | | | | | | | | | |
| Amyloid | INAWLF | Joined | Ure2p | 0.96 | −3.99717 | 56.83316667 | 79.92789987 | ● | ● | | ● |
| Amyloid | LVMELD | CORDAX | Ure2p | 0.91 | −1.07495 | 0 | 0.0011195 | ● | ● | | ● |
| Amyloid | IGINIK | CORDAX | Ure2p | 0.93 | −4.56071 | 0.2325 | 0.036706267 | — | — | ● | ● |
| Amyloid | EVYKWT | CORDAX | Ure2p | 0.88 | −2.1292 | 0 | 0 | — | ● | | ● |
| Amyloid | SYVLQT | CORDAX | Semenogelin | 0.96 | −2.52896 | 0.145333333 | 0.8448973 | ● | ● | | ● |
| Amyloid | LLVYNK | CORDAX | Semenogelin | 0.97 | −3.95159 | 2.455 | 3.488801667 | — | — | ● | ● |
| Non- | LHYGEN | CORDAX | Semenogelin | 0.92 | 0.22706 | 0 | 0 | — | — | — | — |
| Amyloid | | | | | | | | | | | |

TABLE 3
CORDAX cross-threading template-matching predictions. CORDAX accurately predicts both the
topology and matching templates for 42.5% of the sequences derived from the structural library.
Highlighted examples indicate that the correct structural template and topology is predicted
even for sequences corresponding to promiscuous templates removed from the library.

| | | | | | | | | |
|---|---|---|---|---|---|---|---|---|
| Structure | | | β-strand | Prediction | Template | Class | Orientation | FibPredictor |
| code | Sequence | Class | orientation | template | sequence | prediction | prediction | Class |

Template/Topology matches

| | | | | | | | | |
|---|---|---|---|---|---|---|---|---|
| 1yjo/1yjp_2 | NNQQNY | 1 | P | 1yjp_2 | NNQQNY | 1 | P | 3 |
| 1yjp_1 | GNNQQN | 1 | P | 2omm_1 | GNNQQN | 1 | P | 5 |
| 2on9 | VQIVYK | 1 | P | 2on9 | VQIVYK | 1 | P | 4 |
| 3dg1_b | SSTNVG | 1 | P | 3dg1_b | SSTNVG | 1 | P | 8 |
| 3ftl_a_1 | VGSNTY | 1 | P | 3ftl_a_1 | VGSNTY | 1 | P | 5 |
| 3fva_b | NNQNTF | 1 | P | 3fva_b | NNQNTF | 1 | P | 6 |
| 3hyd_b_1 | VEALYL | 1 | P | 3hyd_b_1 | VEALYL | 1 | P | 6 |
| 3hyd_b_2 | LVEALY | 1 | P | 3hyd_b_2 | LVEALY | 1 | P | 6 |
| 3q2x_b | NKGAII | 1 | P | 3q2x_b | NKGAII | 1 | P | 6 |
| 4nio_a_1 | VTGIAQ | 1 | P | 4nio_a_1 | VTGIAQ | 1 | P | 7 |
| 4nip_a_2 | GVIGIA | 1 | P | 4nip_a_2 | GVIGIA | 1 | P | 7 |
| 5vos_a_3 | SNKGAI | 1 | P | 5vos_a_3 | SNKGAI | 1 | P | 7 |
| 2ol9 | SNQNNF | 2 | P | 2ol9 | SNQNNF | 2 | P | 4 |
| 5e5x | ANFLVH | 2 | P | 5e5x | ANFLVH | 2 | P | 1 |
| 2onv_a | GGVVIA | 4 | P | 2onv_a | GGVVIA | 4 | P | 6 |
| 3fpo | HSSNNF | 4 | P | 3fpo | HSSNNF | 4 | P | 7 |
| 3loz | LSFSKD | 5 | AP | 3loz | LSFSKD | 5 | AP | 1 |
| 3fth_2 | FLVHSS | 6 | AP | 3fth_2 | FLVHSS | 6 | AP | 2 |
| 4w67_2 | YVLGSA | 6 | AP | 4w67_2 | YVLGSA | 6 | AP | 5 |
| 4w71_2 | YLLGSA | 6 | AP | 4w71_2 | YLLGSA | 6 | AP | 5 |
| 6cfh_4 | MMGMLA | 6 | AP | 6cfh_4 | MMGMLA | 6 | AP | 6 |
| 3fr1/3fth_1 | NFLVHS | 6/7 | AP | 3fth_1 | NFLVHS | 6 | AP | 8 |
| 2omp | LYQLEN | 7 | AP | 2omp | LYQLEN | 7 | AP | 1 |
| 2y2a | KLVFFA | 7 | AP | 2y2a | KLVFFA | 7 | AP | 7 |
| 4w5l_1 | GGYLLG | 7 | AP | 4w5l_1 | GGYLLG | 7 | AP | 1 |
| 4w5m_1 | GGYMLG | 7 | AP | 4w5m_1 | GGYMLG | 7 | AP | 7 |
| 4w5m_2 | GYMLGS | 7 | AP | 4w5m_2 | GYMLGS | 7 | AP | 5 |
| 4w5p_1 | GGYVLG | 7 | AP | 4w5p_1 | GGYVLG | 7 | AP | 1 |
| 2okz | MVGGVV | 8 | AP | 2okz | MVGGVV | 8 | AP | 5 |
| 4wbv | GYVLGS | 8 | AP | 4wbv | GYVLGS | 8 | AP | 7 |

TABLE 4
CORDAX template-mismatch predictions. Both template and topology-
defined mismatches show predominant sequence homology.

| | | | | | | | | |
|---|---|---|---|---|---|---|---|---|
| Structure | | | β-strand | | Template | Class | Orientation | FibPredictor |
| code | Sequence | Class | orientation | Prediction | sequence | prediction | prediction | Class |

Sequence homology - dependent topology matches

| | | | | | | | | |
|---|---|---|---|---|---|---|---|---|
| 3dgj_1 | NFGAIL | 1 | P | 3q2x_a | NKGAII | 1 | P | 5 |
| 3dgj_2 | NNFGAI | 1 | P | 5vos_a_3 | SNKGAI | 1 | P | 5 |
| 3ppd_a | GGVLVN | 1 | P | 4r0u_a_1 | GVTAVA | 1 | P | 1 |
| 4nio_b_2 | GVTGIA | 1 | P | 4nip_a_2 | GVIGIA | 1 | P | 5 |
| 4nip_b_1 | VIGIAQ | 1 | P | 4nio_a_1 | VTGIAQ | 1 | P | 7 |
| 4r0u_b_2 | GVTAVA | 1 | P | 4nip_a_2 | GVIGIA | 1 | P | 5 |
| 4rik_b_1 | AVVTGV | 1 | P | 5vos_b_6 | GAIIGL | 1 | P | 7 |
| 4znn_b_3 | VHGVTT | 1 | P | 4np8 | VQIVYK | 1 | P | 8 |
| 5vos_b_6 | GAIIGL | 1 | P | 5wkd_b_1 | GNNQGS | 1 | P | 5 |
| 5wkd_a_2 | NNQGSN | 1 | P | 3fva_b | NNQNTF | 1 | P | 1 |
| 4w5l_2 | GYLLGS | 7 | AP | 4w5m_2 | GYMLGS | 7 | AP | 8 |

Sequence homology - independent topology matches

| | | | | | | | | |
|---|---|---|---|---|---|---|---|---|
| 4r0p_b | IFQINS | 1 | P | 4nio_a_1 | VTGIAQ | 1 | P | 1 |
| 4r0w_a_1 | VTGVTA | 1 | P | 6cb9_b | AALQSS | 1 | P | 6 |
| 4znn_a_4 | HGVTTV | 1 | P | 3fva_b | NNQNTF | 1 | P | 1 |
| 5vos_b_5 | KGAIIG | 1 | P | 6cb9_a | AALQSS | 1 | P | 5 |
| 6cb9_b | AALQSS | 1 | P | 3fva_b | NNQNTF | 1 | P | 1 |

Sequence homology - dependent topology mismatches

| | | | | | | | | |
|---|---|---|---|---|---|---|---|---|
| 2onw | SSTSAA | 1 | P | 3loz | LSFSKD | 5 | AP | 6 |
| 3ftl_b_2 | NVGSNT | 1 | P | 2ona | MVGGVV | 8 | AP | 7 |
| 4rik_a_4 | TGVTAV | 1 | P | 2onv_a | GGVVIA | 4 | P | 8 |
| 5whp | NFGTFS | 1 | P | 3fth_1 | NFLVHS | 6 | AP | 1 |
| 4rp6_1 | LTIITL | 2 | P | 3hyd_b_1 | VEALYL | 1 | P | 5 |
| 2m5n_3 | IAALLS | 4 | P | 6cb9_b | AALQSS | 1 | P | 5 |
| 3sgs | GDVIEV | 4 | P | 4rik_b_1 | AVVTGV | 1 | P | 8 |
| 5e5c | VQIINK | 4 | P | 2on9 | VQIVYK | 1 | P | 5 |
| 2y3l_2 | VGGVVI | 7 | AP | 4r0w_a_1 | VTGVTA | 1 | P | 5 |
| 6cew | AMMAAA | 7 | AP | 6cfh_4 | MMGMLA | 6 | AP | 8 |
| 5e5v_2 | FGAILS | 8 | AP | 5vos_a_5 | KGAIIG | 1 | P | 7 |

Sequence homology - independent topology mismatches

| | | | | | | | | |
|---|---|---|---|---|---|---|---|---|
| 4r0w_a_2 | VVTGVT | 1 | P | 6cfh_4 | MMGMLA | 6 | AP | 7 |
| 4znn_a_5 | GVTTVA | 1 | P | 2y2a | KLVFFA | 7 | AP | 5 |
| 5wia | GNNSYS | 1 | P | 2y2a | KLVFFA | 7 | AP | 8 |
| 2y3j | AIIGLM | 2 | P | 3hyd_b_1 | VEALYL | 1 | P | 5 |
| 4nin_1 | DSVISL | 2 | P | 3fva_a | NNQNTF | 1 | P | 7 |
| 4nin_2 | SVISLS | 2 | P | 3hyd_b_1 | VEALYL | 1 | P | 5 |
| 4rp6_2 | TIITLE | 2 | P | 2y2a | KLVFFA | 7 | AP | nd |
| 4xfo | TAVVTN | 2 | P | 3fva_b | NNQNTF | 1 | P | 8 |
| 5w52_5 | KGISVH | 2 | P | 4rik_b_2 | VVTGVT | 1 | P | 1 |
| 2m5n_2 | TIAALL | 4 | P | 5vos_b_6 | GAIIGL | 1 | P | 7 |
| 2m5n_4 | AALLSP | 4 | P | 6cfh_4 | MMGMLA | 6 | AP | 7 |
| 5n9i | GVVTSE | 4 | P | 6cfh_4 | MMGMLA | 6 | AP | 7 |
| 5wiq_1 | GFNGGF | 4 | P | 3fva_a | NNQNTF | 1 | P | 8 |
| 5wkb | NFGEFS | 4 | P | 3fth_2 | FLVHSS | 6 | AP | 6 |
| 3nve | MMHFGN | 6 | AP | 4nip_b_1 | VIGIAQ | 1 | P | 5 |
| 5e5z | LVHSSN | 7 | AP | 3fva_a | NNQNTF | 1 | P | 1 |

TABLE 5
Performance on regional detection of aggregation prone
segments in the reg33 dataset using the annotation described
in Tsolis AC et al (2013) PloS one 8, e54175.

| Predictor | Sensitivity (%) | Specificity (%) | MCC |
|---|---|---|---|
| CORDAX | 25.87 | 89.49 | 0.17 |
| WALTZ | 56.43 | 65.42 | 0.16 |
| AGGRESCAN | 35.37 | 79.26 | 0.13 |
| SALSA | 69.63 | 47.44 | 0.13 |
| MILAMP | 62.33 | 62.80 | 0.19 |
| 3D profile | 17.95 | 87.53 | 0.06 |
| TANGO | 13.67 | 95.57 | 0.14 |
| Zyggregator | 28.73 | 86.31 | 0.15 |
| AMYLPRED2 | 38.30 | 83.73 | 0.20 |
| PAFIG | 51.75 | 71.43 | 0.18 |
| FISH Amyloid | 13.73 | 93.68 | 0.10 |
| Fold Amyloid | 20.71 | 86.97 | 0.08 |
| PASTA 2.0 (High sensitivity) | 40.87 | 84.95 | 0.24 |
| MetAmyl (High Specificity) | 39.05 | 83.14 | 0.19 |

### REFERENCES

1 Benson, M. D. et al. Amyloid nomenclature 2018: recommendations by the International Society of Amyloidosis (ISA) nomenclature committee. Amyloid: the international journal of experimental and clinical investigation: the official journal of the International Society of Amyloidosis 25, 215-219, doi:10.1080/13506129.2018.1549825 (2018).
2 Chiti, F. & Dobson, C. M. Protein Misfolding, Amyloid Formation, and Human Disease: A Summary of Progress Over the Last Decade. Annual review of biochemistry 86, 27-68, doi:10.1146/annurev-biochem-061516-045115 (2017).
3 Pham, C. L., Kwan, A. H. & Sunde, M. Functional amyloid: widespread in Nature, diverse in purpose. Essays in biochemistry 56, 207-219, doi:10.1042/bse0560207 (2014).
4 Stefani, M. & Dobson, C. M. Protein aggregation and aggregate toxicity: new insights into protein folding, misfolding diseases and biological evolution. Journal of molecular medicine 81, 678-699, doi:10.1007/s00109-003-0464-5 (2003).
5 Lopez de la Paz, M. & Serrano, L. Sequence determinants of amyloid fibril formation. Proceedings of the National Academy of Sciences of the United States of America 101, 87-92, doi:10.1073/pnas.2634884100 (2004).
6 Chiti, F., Stefani, M., Taddei, N., Ramponi, G. & Dobson, C. M. Rationalization of the effects of mutations on peptide and protein aggregation rates. Nature 424, 805-808, doi:10.1038/nature01891 (2003).
7 Fernandez-Escamilla, A. M., Rousseau, F., Schymkowitz, J. & Serrano, L. Prediction of sequence-dependent and mutational effects on the aggregation of peptides and proteins. Nature biotechnology 22, 1302-1306, doi:10.1038/nbt1012 (2004).
8 Pawar, A. P. et al. Prediction of “aggregation-prone” and “aggregation-susceptible” regions in proteins associated with neurodegenerative diseases. Journal of molecular biology 350, 379-392, doi:10.1016/j.jmb.2005.04.016 (2005).
9 de Groot, N. S., Castillo, V., Grana-Montes, R. & Ventura, S. AGGRESCAN: method, application, and perspectives for drug design. Methods in molecular biology 819, 199-220, doi:10.1007/978-1-61779-465-0_14 (2012).
10 Tartaglia, G. G. et al. Prediction of aggregation-prone regions in structured proteins. Journal of molecular biology 380, 425-436, doi:10.1016/j.jmb.2008.05.013 (2008).
11 Beerten, J., Schymkowitz, J. & Rousseau, F. Aggregation prone regions and gatekeeping residues in protein sequences. Current topics in medicinal chemistry 12, 2470-2478, doi:10.2174/1568026611212220003 (2012).
12 Buck, P. M., Kumar, S. & Singh, S. K. On the role of aggregation prone regions in protein evolution, stability, and enzymatic catalysis: insights from diverse analyses. PLoS computational biology 9, e1003291, doi:10.1371/journal.pcbi.1003291 (2013).
13 Castillo, V. & Ventura, S. Amyloidogenic regions and interaction surfaces overlap in globular proteins related to conformational diseases. PLoS computational biology 5, e1000476, doi:10.1371/journal.pcbi.1000476 (2009).
14 Dobson, C. M. Protein folding and misfolding. Nature 426, 884-890, doi:10.1038/nature02261 (2003).
15 Mishra, A., Ranganathan, S., Jayaram, B. & Sattar, A. Role of solvent accessibility for aggregation-prone patches in protein folding. Sci Rep 8, 12896, doi:10.1038/s41598-018-31289-6 (2018).
16 Alberti, S., Gladfelter, A. & Mittag, T. Considerations and Challenges in Studying Liquid-Liquid Phase Separation and Biomolecular Condensates. Cell 176, 419-434, doi:10.1016/j.ce11.2018.12.035 (2019).
17 Mohammadi, P. et al. Phase transitions as intermediate steps in the formation of molecularly engineered protein fibers. Communications biology 1, 86, doi:10.1038/s42003-018-0090-y (2018).
18 Schmidt, H. B., Barreau, A. & Rohatgi, R. Phase separation-deficient TDP43 remains functional in splicing. Nature communications 10, 4890, doi:10.1038/s41467-019-12740-2 (2019).
19 Hamodrakas, S. J. Protein aggregation and amyloid fibril formation prediction software from primary sequence: towards controlling the formation of bacterial inclusion bodies. The FEBS journal 278, 2428-2435, doi:10.1111/j.1742-4658.2011.08164.x (2011).
20 Gasior, P. & Kotulska, M. FISH Amyloid—a new method for finding amyloidogenic segments in proteins based on site specific co-occurrence of aminoacids. BMC bioinformatics 15, 54, doi:10.1186/1471-2105-15-54 (2014).
21 Maurer-Stroh, S. et al. Exploring the sequence determinants of amyloid structure using position-specific scoring matrices. Nature methods 7, 237-242, doi:10.1038/nmeth.1432 (2010).
22 Thangakani, A. M., Kumar, S., Nagarajan, R., Velmurugan, D. & Gromiha, M. M. GAP: towards almost 100 percent prediction for beta-strand-mediated aggregating peptides with distinct morphologies. Bioinformatics 30, 1983-1990, doi:10.1093/bioinformatics/btu167 (2014).
23 Walsh, I., Seno, F., Tosatto, S. C. & Trovato, A. PASTA 2.0: an improved server for protein aggregation prediction. Nucleic acids research 42, W301-307, doi:10.1093/nar/gku399 (2014).
24 Emily, M., Talvas, A. & Delamarche, C. MetAmyl: a METa-predictor for AMYLoid proteins. PloS one 8, e79722, doi:10.1371/journal.pone.0079722 (2013).
25 Tsolis, A. C., Papandreou, N. C., Iconomidou, V. A. & Hamodrakas, S. J. A consensus method for the prediction of ‘aggregation-prone’ peptides in globular proteins. PloS one 8, e54175, doi:10.1371/journal.pone.0054175 (2013).
26 Kim, C., Choi, J., Lee, S. J., Welsh, W. J. & Yoon, S. NetCSSP: web application for predicting chameleon sequences and amyloid fibril formation. Nucleic acids research 37, W469-473, doi:10.1093/nar/gkp351 (2009).
27 Yoon, S. & Welsh, W. J. Detecting hidden sequence propensity for amyloid fibril formation. Protein science: a publication of the Protein Society 13, 2149-2160, doi:10.1110/ps.04790604 (2004).
28 Bondarev, S. A., Bondareva, O. V., Zhouravleva, G. A. & Kajava, A. V. BetaSerpentine: a bioinformatics tool for reconstruction of amyloid structures. Bioinformatics 34, 599-608, doi:10.1093/bioinformatics/btx629 (2018).
29 Thompson, M. J. et al. The 3D profile method for identifying fibril-forming segments of proteins. Proceedings of the National Academy of Sciences of the United States of America 103, 4074-4078, doi:10.1073/pnas.0511295103 (2006).
30 Schymkowitz, J. et al. The FoldX web server: an online force field. Nucleic acids research 33, W382-388, doi:10.1093/nar/gki387 (2005).
31 Sawaya, M. R. et al. Atomic structures of amyloid cross-beta spines reveal varied steric zippers. Nature 447, 453-457, doi:10.1038/nature05695 (2007).
32 Louros, N. et al. WALTZ-DB 2.0: an updated database containing structural information of experimentally determined amyloid-forming peptides. Nucleic acids research, doi:10.1093/nar/gkz758 (2019).
33 Sing, T., Sander, O., Beerenwinkel, N. & Lengauer, T. ROCR: visualizing classifier performance in R. Bioinformatics 21, 3940-3941, doi:10.1093/bioinformatics/bti623 (2005).
34 Munir, F., Gull, S., Asif, A. & Minhas, F. MILAMP: Multiple Instance Prediction of Amyloid Proteins. IEEE/ACM Trans Comput Biol Bioinform, doi:10.1109/TCBB.2019.2936846 (2019).
35 Iconomidou, V. A., Leontis, A., Hoenger, A. & Hamodrakas, S. J. Identification of a novel ‘aggregation-prone’/‘amyloidogenic determinant’ peptide in the sequence of the highly amyloidogenic human calcitonin. FEBS letters 587, 569-574, doi:10.1016/j.febslet.2013.01.031 (2013).
36 Tsiolaki, P. L., Louros, N. N., Hamodrakas, S. J. & Iconomidou, V. A. Exploring the ‘aggregation-prone’ core of human Cystatin C: A structural study. Journal of structural biology 191, 272-280, doi:10.1016/j.jsb.2015.07.013 (2015).
37 Saelices, L. et al. Uncovering the Mechanism of Aggregation of Human Transthyretin. The Journal of biological chemistry 290, 28932-28943, doi:10.1074/jbc.M115.659912 (2015).
38 Baxa, U. et al. Characterization of beta-sheet structure in Ure2p1-89 yeast prion fibrils by solid-state nuclear magnetic resonance. Biochemistry 46, 13149-13162, doi:10.1021/bi700826b (2007).
39 Gross, M. et al. Formation of amyloid fibrils by peptides derived from the bacterial cold shock protein CspB. Protein science: a publication of the Protein Society 8, 1350-1357, doi:10.1110/ps.8.6.1350 (1999).
40 Louros, N. N. et al. Chameleon ‘aggregation-prone’ segments of apoA-I: A model of amyloid fibrils formed in apoA-I amyloidosis. International journal of biological macromolecules 79, 711-718, doi:10.1016/j.ijbiomac.2015.05.032 (2015).
41 Van Melckebeke, H. et al. Atomic-resolution three-dimensional structure of HET-s (218-289) amyloid fibrils by solid-state NMR spectroscopy. Journal of the American Chemical Society 132, 13765-13775, doi:10.1021/ja104213j (2010).
42 Rauscher, S., Baud, S., Miao, M., Keeley, F. W. & Pomes, R. Proline and glycine control protein self-organization into elastomeric or amyloid fibrils. Structure 14, 1667-1676, doi:10.1016/j.str.2006.09.008 (2006).
43 Tsiolaki, P. L., Louros, N. N. & Iconomidou, V. A. Hexapeptide Tandem Repeats Dictate the Formation of Silkmoth Chorion, a Natural Protective Amyloid. Journal of molecular biology 430, 3774-3783, doi:10.1016/j.jmb.2018.06.042 (2018).
44 Chernoff, Y. O. Amyloidogenic domains, prions and structural inheritance: rudiments of early life or recent acquisition? Current opinion in chemical biology 8, 665-671, doi:10.1016/j.cbpa.2004.09.002 (2004).
45 Greenwald, J., Friedmann, M. P. & Riek, R. Amyloid Aggregates Arise from Amino Acid Condensations under Prebiotic Conditions. Angewandte Chemie 55, 11609-11613, doi:10.1002/anie.201605321 (2016).
46 Martin, E. W. & Mittag, T. Relationship of Sequence and Phase Separation in Protein Low-Complexity Regions. Biochemistry 57, 2478-2487, doi:10.1021/acs.biochem.8b00008 (2018).
47 Rousseau, F., Serrano, L. & Schymkowitz, J. W. How evolutionary pressure against protein aggregation shaped chaperone specificity. Journal of molecular biology 355, 1037-1047, doi:10.1016/j.jmb.2005.11.035 (2006).
48 Gazit, E. Self assembly of short aromatic peptides into amyloid fibrils and related nanostructures. Prion 1, 32-35, doi:10.4161/pri.1.1.4095 (2007).
49 Tabatabaei Ghomi, H., Topp, E. M. & Lill, M. A. Fibpredictor: a computational method for rapid prediction of amyloid fibril structures. Journal of molecular modeling 22, 206, doi:10.1007/s00894-016-3066-1 (2016).
50 Landau, M. et al. Towards a pharmacophore for amyloid. PLoS biology 9, e1001080, doi:10.1371/journal.pbio.1001080 (2011).
51 Berhanu, W. M. & Masunov, A. E. Alternative packing modes leading to amyloid polymorphism in five fragments studied with molecular dynamics. Biopolymers 98, 131-144, doi:10.1002/bip.21731 (2012).
52 Yu, L., Lee, S. J. & Yee, V. C. Crystal Structures of Polymorphic Prion Protein beta1 Peptides Reveal Variable Steric Zipper Conformations. Biochemistry 54, 3640-3648, doi:10.1021/acs.biochem.5b00425 (2015).
53 Tycko, R. Amyloid polymorphism: structural basis and neurobiological relevance. Neuron 86, 632-645, doi:10.1016/j.neuron.2015.03.017 (2015).
54 Close, W. et al. Physical basis of amyloid fibril polymorphism. Nature communications 9, 699, doi:10.1038/541467-018-03164-5 (2018).
55 Perov, S. et al. Structural Insights into Curli CsgA Cross-beta Fibril Architecture Inspire Repurposing of Anti-amyloid Compounds as Anti-biofilm Agents. PLoS pathogens 15, e1007978, doi:10.1371/journal.ppat.1007978 (2019).
56 Do, T. D. et al. Distal amyloid beta-protein fragments template amyloid assembly. Protein science a publication of the Protein Society 27, 1181-1190, doi:10.1002/pro.3375 (2018).
57 Nannenga, B. L. & Gonen, T. The cryo-EM method microcrystal electron diffraction (MicroED). Nature methods 16, 369-379, doi:10.1038/541592-019-0395-x (2019).
58 Fandrich, M. et al. Amyloid fibril polymorphism: a challenge for molecular imaging and therapy. Journal of internal medicine 283, 218-237, doi:10.1111/joim.12732 (2018).
59 Tycko, R. Molecular Structure of Aggregated Amyloid-beta: Insights from Solid-State Nuclear Magnetic Resonance. Cold Spring Harbor perspectives in medicine 6, doi:10.1101/cshperspect.a024083 (2016).
60 Gallardo, R., Ranson, N. A. & Radford, S. E. Amyloid structures: much more than just a cross-beta fold. Curr Opin Struct Biol 60, 7-16, doi:10.1016/j.sbi.2019.09.001 (2020).
61 Lu, J. et al. Structure-Based Peptide Inhibitor Design of Amyloid-beta Aggregation. Frontiers in molecular neuroscience 12, 54, doi:10.3389/fnmo1.2019.00054 (2019).
62 Seidler, P. M. et al. Structure-based inhibitors halt prion-like seeding by Alzheimer's disease- and tauopathy-derived brain tissue samples. The Journal of biological chemistry, doi:10.1074/jbc.RA119.009688 (2019).
63 Sivanesam, K. et al. Peptide Inhibitors of the amyloidogenesis of IAPP: verification of the hairpin-binding geometry hypothesis. FEBS letters 590, 2575-2583, doi:10.1002/1873-3468.12261 (2016).
64 Mitraki, A. Protein aggregation from inclusion bodies to amyloid and biomaterials. Advances in protein chemistry and structural biology 79, 89-125, doi:10.1016/S1876-1623(10)79003-9 (2010).
65 Khodaparast, L. et al. Aggregating sequences that occur in many proteins constitute weak spots of bacterial proteostasis. Nature communications 9, 866, doi:10.1038/s41467-018-03131-0 (2018).
66 Pedegrosa, F. et al. Scikit-learn: Machine Learning in Python. JMLR 12, 2825-2830 (2011).
67 Chen, M., Schafer, N. P., Zheng, W. & Wolynes, P. G. The Associative Memory, Water Mediated, Structure and Energy Model (AWSEM)-Amylometer: Predicting Amyloid Propensity and Fibril Topology Using an Optimized Folding Landscape Model. ACS chemical neuroscience 9, 1027-1039, doi:10.1021/acschemneuro.7b00436 (2018).
68 Varadi, M., De Baets, G., Vranken, W. F., Tompa, P. & Pancsa, R. AmyPro: a database of proteins with validated amyloidogenic regions. Nucleic acids research 46, D387-D392, doi:10.1093/nar/gkx950 (2018).
69 Wozniak, P. P. & Kotulska, M. AmyLoad: website dedicated to amyloidogenic protein fragments. Bioinformatics 31, 3395-3397, doi:10.1093/bioinformatics/btv375 (2015).
70 Niu, M., Li, Y., Wang, C. & Han, K. RFAmyloid: A Web Server for Predicting Amyloid Proteins. International journal of molecular sciences 19, doi:10.3390/ijms19072071 (2018).
71 Sankar, K., Krystek, S. R., Jr., Carl, S. M., Day, T. & Maier, J. K. X. AggScore: Prediction of aggregation-prone regions in proteins based on the distribution of surface patches. Proteins 86, 1147-1156, doi:10.1002/prot.25594 (2018).
72 Tao, P., Wang, R. & Lai, L. Calculating Partition Coefficients of Peptides by the Addition Method. Molecular modeling annual 5, 189-195, doi:10.1007/s008940050118 (1999).
73 Krieger, E. & Vriend, G. YASARA View—molecular graphics for all devices—from smartphones to workstations. Bioinformatics 30, 2981-2982, doi:10.1093/bioinformatics/btu426 (2014).
74 van der Maaten, L. J. P. & Hinton, G. E. Visualizing High-Dimensional Data Using t-SNE. Journal of Machine Learning Research 9, 2579-2605 (2008).

1. A method for identifying at least one aggregation prone region (APR) present in a target protein, the method comprising:
querying a machine learning engine for an APR present in a target protein, the machine learning engine having been trained using a first library comprising experimentally defined amyloidogenic sequences from amyloid-forming proteins wherein the amyloidogenic sequences are modelled on the backbone structures of a second library of amyloid fibril core structures and wherein the thermodynamic stability of each model is calculated by a Force Field and the calculations are introduced into a logistic regression model to score the aggregation propensity and,
obtaining at least one candidate APR sequence.

2. The method according to claim 1, wherein querying comprises:
fragmenting the target protein into hexapeptides using a sliding window process, modelling the hexapeptides on the backbone of the second library, calculating the thermodynamic stability for each sequence using a Force Field and feeding the data into the logistic regression model.

3. The method according to claim 1 wherein the Force Field is FoldX.

4. A computer-readable storage medium which stores computer-executable instruction that, when executed by at least one processor, cause the processor to perform the method of claim 1.

5. An apparatus comprising control circuitry configured to perform the method of claim 1.


SEQUENCE LISTING (182 sequences)

SEQ ID NO: 1 - protein, 6 aa, Artificial Sequence
  Cordax-predicted peptide screen
SVDYEV

SEQ ID NO: 2 - protein, 6 aa, Artificial Sequence
  Cordax-predicted peptide screen
IGVVGW

SEQ ID NO: 3 - protein, 6 aa, Artificial Sequence
  Cordax-predicted peptide screen
NFSIRY

SEQ ID NO: 4 - protein, 6 aa, Artificial Sequence
  Cordax-predicted peptide screen
VNFSEF

SEQ ID NO: 5 - protein, 6 aa, Artificial Sequence
  Cordax-predicted peptide screen
SEFSKK

SEQ ID NO: 6 - protein, 6 aa, Artificial Sequence
  Cordax-predicted peptide screen
AFFLFC

SEQ ID NO: 7 - protein, 6 aa, Artificial Sequence
  Cordax-predicted peptide screen
LSSYWE

SEQ ID NO: 8 - protein, 6 aa, Artificial Sequence
  Cordax-predicted peptide screen
NLYEKT

SEQ ID NO: 9 - protein, 6 aa, Artificial Sequence
  Cordax-predicted peptide screen
VYVDVL

SEQ ID NO: 10 - protein, 6 aa, Artificial Sequence
  Cordax-predicted peptide screen
LNLKLL

SEQ ID NO: 11 - protein, 6 aa, Artificial Sequence
  Cordax-predicted peptide screen
LLDNWD

SEQ ID NO: 12 - protein, 6 aa, Artificial Sequence
  Cordax-predicted peptide screen
MDVFMK

SEQ ID NO: 13 - protein, 6 aa, Artificial Sequence
  Cordax-predicted peptide screen
IQVYSR

SEQ ID NO: 14 - protein, 6 aa, Artificial Sequence
  Cordax-predicted peptide screen
LCSTFC

SEQ ID NO: 15 - protein, 6 aa, Artificial Sequence
  Cordax-predicted peptide screen
LNFLKK

SEQ ID NO: 16 - protein, 6 aa, Artificial Sequence
  Cordax-predicted peptide screen
KTVYQH

SEQ ID NO: 17 - protein, 6 aa, Artificial Sequence
  Cordax-predicted peptide screen
TKVIPY

SEQ ID NO: 18 - protein, 6 aa, Artificial Sequence
  Cordax-predicted peptide screen
MVVEHP

SEQ ID NO: 19 - protein, 6 aa, Artificial Sequence
  Cordax-predicted peptide screen
AYVILK

SEQ ID NO: 20 - protein, 6 aa, Artificial Sequence
  Cordax-predicted peptide screen
GWFLGW

SEQ ID NO: 21 - protein, 6 aa, Artificial Sequence
  Cordax-predicted peptide screen
YDLHYW

SEQ ID NO: 22 - protein, 6 aa, Artificial Sequence
  Cordax-predicted peptide screen
IFTVQL

SEQ ID NO: 23 - protein, 6 aa, Artificial Sequence
  Cordax-predicted peptide screen
FLGYFK

SEQ ID NO: 24 - protein, 6 aa, Artificial Sequence
  Cordax-predicted peptide screen
MSVSLV

SEQ ID NO: 25 - protein, 6 aa, Artificial Sequence
  Cordax-predicted peptide screen
EDCFIL

SEQ ID NO: 26 - protein, 6 aa, Artificial Sequence
  Cordax-predicted peptide screen
KIFVWK

SEQ ID NO: 27 - protein, 6 aa, Artificial Sequence
  Cordax-predicted peptide screen
DFITKM

SEQ ID NO: 28 - protein, 6 aa, Artificial Sequence
  Cordax-predicted peptide screen
YIILYN

SEQ ID NO: 29 - protein, 6 aa, Artificial Sequence
  Cordax-predicted peptide screen
IIYNWQ

SEQ ID NO: 30 - protein, 6 aa, Artificial Sequence
  Cordax-predicted peptide screen
MIIYKG

SEQ ID NO: 31 - protein, 6 aa, Artificial Sequence
  Cordax-predicted peptide screen
NDAFVL

SEQ ID NO: 32 - protein, 6 aa, Artificial Sequence
  Cordax-predicted peptide screen
AAYLWV

SEQ ID NO: 33 - protein, 6 aa, Artificial Sequence
  Cordax-predicted peptide screen
FVIEEV

SEQ ID NO: 34 - protein, 6 aa, Artificial Sequence
  Cordax-predicted peptide screen
QVFVWV

SEQ ID NO: 35 - protein, 6 aa, Artificial Sequence
  Cordax-predicted peptide screen
GIVAGA

SEQ ID NO: 36 - protein, 6 aa, Artificial Sequence
  Cordax-predicted peptide screen
DCFEYV

SEQ ID NO: 37 - protein, 6 aa, Artificial Sequence
  Cordax-predicted peptide screen
FEYVQL

SEQ ID NO: 38 - protein, 6 aa, Artificial Sequence
  Cordax-predicted peptide screen
ILLLFE

SEQ ID NO: 39 - protein, 6 aa, Artificial Sequence
  Cordax-predicted peptide screen
DLVVFE

SEQ ID NO: 40 - protein, 6 aa, Artificial Sequence
  Cordax-predicted peptide screen
AEIEIE

SEQ ID NO: 41 - protein, 6 aa, Artificial Sequence
  Cordax-predicted peptide screen
ASLTIL

SEQ ID NO: 42 - protein, 6 aa, Artificial Sequence
  Cordax-predicted peptide screen
YQLVLQ

SEQ ID NO: 43 - protein, 6 aa, Artificial Sequence
  Cordax-predicted peptide screen
STVISY

SEQ ID NO: 44 - protein, 6 aa, Artificial Sequence
  Cordax-predicted peptide screen
VNIELL

SEQ ID NO: 45 - protein, 6 aa, Artificial Sequence
  Cordax-predicted peptide screen
IQIHHY

SEQ ID NO: 46 - protein, 6 aa, Artificial Sequence
  Cordax-predicted peptide screen
QIIEIE

SEQ ID NO: 47 - protein, 6 aa, Artificial Sequence
  Cordax-predicted peptide screen
GVIHYI

SEQ ID NO: 48 - protein, 6 aa, Artificial Sequence
  Cordax-predicted peptide screen
LNSVFK

SEQ ID NO: 49 - protein, 6 aa, Artificial Sequence
  Cordax-predicted peptide screen
LYHGQT

SEQ ID NO: 50 - protein, 6 aa, Artificial Sequence
  Cordax-predicted peptide screen
TLFTMD

SEQ ID NO: 51 - protein, 6 aa, Artificial Sequence
  Cordax-predicted peptide screen
VYTVFA

SEQ ID NO: 52 - protein, 6 aa, Artificial Sequence
  Cordax-predicted peptide screen
HYYAVA

SEQ ID NO: 53 - protein, 6 aa, Artificial Sequence
  Cordax-predicted peptide screen
GDVAFI

SEQ ID NO: 54 - protein, 6 aa, Artificial Sequence
  Cordax-predicted peptide screen
LLFKDS

SEQ ID NO: 55 - protein, 6 aa, Artificial Sequence
  Cordax-predicted peptide screen
YFTAIQ

SEQ ID NO: 56 - protein, 6 aa, Artificial Sequence
  Cordax-predicted peptide screen
GWNIPM

SEQ ID NO: 57 - protein, 6 aa, Artificial Sequence
  Cordax-predicted peptide screen
MDKVER

SEQ ID NO: 58 - protein, 6 aa, Artificial Sequence
  Cordax-predicted peptide screen
YVAGIT

SEQ ID NO: 59 - protein, 6 aa, Artificial Sequence
  Cordax-predicted peptide screen
CEFLRK

SEQ ID NO: 60 - protein, 6 aa, Artificial Sequence
  Cordax-predicted peptide screen
LQVDLG

SEQ ID NO: 61 - protein, 6 aa, Artificial Sequence
  Cordax-predicted peptide screen
FDKFKH

SEQ ID NO: 62 - protein, 6 aa, Artificial Sequence
  Cordax-predicted peptide screen
MFSEFD

SEQ ID NO: 63 - protein, 6 aa, Artificial Sequence
  Cordax-predicted peptide screen
LIVSIL

SEQ ID NO: 64 - protein, 6 aa, Artificial Sequence
  Cordax-predicted peptide screen
LYHLVT

SEQ ID NO: 65 - protein, 6 aa, Artificial Sequence
  Cordax-predicted peptide screen
MELIVS

SEQ ID NO: 66 - protein, 6 aa, Artificial Sequence
  Cordax-predicted peptide screen
LHCLRR

SEQ ID NO: 67 - protein, 6 aa, Artificial Sequence
  Cordax-predicted peptide screen
QVYYRP

SEQ ID NO: 68 - protein, 6 aa, Artificial Sequence
  Cordax-predicted peptide screen
GYLTIR

SEQ ID NO: 69 - protein, 6 aa, Artificial Sequence
  Cordax-predicted peptide screen
KLFNRD

SEQ ID NO: 70 - protein, 6 aa, Artificial Sequence
  Cordax-predicted peptide screen
FHVKYR

SEQ ID NO: 71 - protein, 6 aa, Artificial Sequence
  Cordax-predicted peptide screen
MLHKEF

SEQ ID NO: 72 - protein, 6 aa, Artificial Sequence
  Cordax-predicted peptide screen
FYAVRL

SEQ ID NO: 73 - protein, 6 aa, Artificial Sequence
  Cordax-predicted peptide screen
SQFIKL

SEQ ID NO: 74 - protein, 6 aa, Artificial Sequence
  Cordax-predicted peptide screen
FSFTIA

SEQ ID NO: 75 - protein, 6 aa, Artificial Sequence
  Cordax-predicted peptide screen
YFHARG

SEQ ID NO: 76 - protein, 6 aa, Artificial Sequence
  Cordax-predicted peptide screen
LIFMGH

SEQ ID NO: 77 - protein, 6 aa, Artificial Sequence
  Cordax-predicted peptide screen
MYVSEM

SEQ ID NO: 78 - protein, 6 aa, Artificial Sequence
  Cordax-predicted peptide screen
VVVNKM

SEQ ID NO: 79 - protein, 6 aa, Artificial Sequence
  Cordax-predicted peptide screen
SNFLRA

SEQ ID NO: 80 - protein, 6 aa, Artificial Sequence
  Cordax-predicted peptide screen
IGYNIK

SEQ ID NO: 81 - protein, 6 aa, Artificial Sequence
  Cordax-predicted peptide screen
TDVVFM

SEQ ID NO: 82 - protein, 6 aa, Artificial Sequence
  Cordax-predicted peptide screen
VDMAMC

SEQ ID NO: 83 - protein, 6 aa, Artificial Sequence
  Cordax-predicted peptide screen
VHIVKL

SEQ ID NO: 84 - protein, 6 aa, Artificial Sequence
  Cordax-predicted peptide screen
INFEFS

SEQ ID NO: 85 - protein, 6 aa, Artificial Sequence
  Cordax-predicted peptide screen
GYTLFS

SEQ ID NO: 86 - protein, 6 aa, Artificial Sequence
  Cordax-predicted peptide screen
FKVAIV

SEQ ID NO: 87 - protein, 6 aa, Artificial Sequence
  Cordax-predicted peptide screen
TIFLDF

SEQ ID NO: 88 - protein, 6 aa, Artificial Sequence
  Cordax-predicted peptide screen
LSIWES

SEQ ID NO: 89 - protein, 6 aa, Artificial Sequence
  Cordax-predicted peptide screen
HLVNKY

SEQ ID NO: 90 - protein, 6 aa, Artificial Sequence
  Cordax-predicted peptide screen
INAWLF

SEQ ID NO: 91 - protein, 6 aa, Artificial Sequence
  Cordax-predicted peptide screen
LVMELD

SEQ ID NO: 92 - protein, 6 aa, Artificial Sequence
  Cordax-predicted peptide screen
IGINIK

SEQ ID NO: 93 - protein, 6 aa, Artificial Sequence
  Cordax-predicted peptide screen
EVYKWT

SEQ ID NO: 94 - protein, 6 aa, Artificial Sequence
  Cordax-predicted peptide screen
SYVLQT

SEQ ID NO: 95 - protein, 6 aa, Artificial Sequence
  Cordax-predicted peptide screen
LLVYNK

SEQ ID NO: 96 - protein, 6 aa, Artificial Sequence
  Cordax-predicted peptide screen
LHYGEN

SEQ ID NO: 97 - protein, 6 aa, Artificial Sequence
  Peptide screen
NNQQNY

SEQ ID NO: 98 - protein, 6 aa, Artificial Sequence
  Cordax peptide screen
GNNQQN

SEQ ID NO: 99 - protein, 6 aa, Artificial Sequence
  Cordax peptide screen
VQIVYK

SEQ ID NO: 100 - protein, 6 aa, Artificial Sequence
  Cordax peptide screen
SSTNVG

SEQ ID NO: 101 - protein, 6 aa, Artificial Sequence
  Cordax peptide screen
VGSNTY

SEQ ID NO: 102 - protein, 6 aa, Artificial Sequence
  Cordax peptide screen
NNQNTF

SEQ ID NO: 103 - protein, 6 aa, Artificial Sequence
  Cordax peptide screen
VEALYL

SEQ ID NO: 104 - protein, 6 aa, Artificial Sequence
  Cordax peptide screen
LVEALY

SEQ ID NO: 105 - protein, 6 aa, Artificial Sequence
  Cordax peptide screen
NKGAII

SEQ ID NO: 106 - protein, 6 aa, Artificial Sequence
  Cordax peptide screen
VTGIAQ

SEQ ID NO: 107 - protein, 6 aa, Artificial Sequence
  Cordax peptide screen
GVIGIA

SEQ ID NO: 108 - protein, 6 aa, Artificial Sequence
  Cordax peptide screen
SNKGAI

SEQ ID NO: 109 - protein, 6 aa, Artificial Sequence
  Cordax peptide screen
SNQNNF

SEQ ID NO: 110 - protein, 6 aa, Artificial Sequence
  Cordax peptide screen
ANFLVH

SEQ ID NO: 111 - protein, 6 aa, Artificial Sequence
  Cordax peptide screen
GGVVIA

SEQ ID NO: 112 - protein, 6 aa, Artificial Sequence
  Cordax peptide screen
HSSNNF

SEQ ID NO: 113 - protein, 6 aa, Artificial Sequence
  Cordax peptide screen
LSFSKD

SEQ ID NO: 114 - protein, 6 aa, Artificial Sequence
  Cordax peptide screen
FLVHSS

SEQ ID NO: 115 - protein, 6 aa, Artificial Sequence
  Cordax peptide screen
YVLGSA

SEQ ID NO: 116 - protein, 6 aa, Artificial Sequence
  Cordax peptide screen
YLLGSA

SEQ ID NO: 117 - protein, 6 aa, Artificial Sequence
  Cordax peptide screen
MMGMLA

SEQ ID NO: 118 - protein, 6 aa, Artificial Sequence
  Cordax peptide screen
NFLVHS

SEQ ID NO: 119 - protein, 6 aa, Artificial Sequence
  Cordax peptide screen
LYQLEN

SEQ ID NO: 120 - protein, 6 aa, Artificial Sequence
  Cordax peptide screen
KLVFFA

SEQ ID NO: 121 - protein, 6 aa, Artificial Sequence
  Cordax peptide screen
GGYLLG

SEQ ID NO: 122 - protein, 6 aa, Artificial Sequence
  Cordax peptide screen
GGYMLG

SEQ ID NO: 123 - protein, 6 aa, Artificial Sequence
  Cordax peptide screen
GYMLGS

SEQ ID NO: 124 - protein, 6 aa, Artificial Sequence
  Cordax peptide screen
GGYVLG

SEQ ID NO: 125 - protein, 6 aa, Artificial Sequence
  Cordax peptide screen
MVGGVV

SEQ ID NO: 126 - protein, 6 aa, Artificial Sequence
  Cordax peptide screen
GYVLGS

SEQ ID NO: 127 - protein, 6 aa, Artificial Sequence
  Cordax peptide screen
NFGAIL

SEQ ID NO: 128 - protein, 6 aa, Artificial Sequence
  Cordax peptide screen
NNFGAI

SEQ ID NO: 129 - protein, 6 aa, Artificial Sequence
  Cordax peptide screen
GGVLVN

SEQ ID NO: 130 - protein, 6 aa, Artificial Sequence
  Cordax peptide screen
GVTAVA

SEQ ID NO: 131 - protein, 6 aa, Artificial Sequence
  Cordax peptide screen
GVTGIA

SEQ ID NO: 132 - protein, 6 aa, Artificial Sequence
  Cordax peptide screen
VIGIAQ

SEQ ID NO: 133 - protein, 6 aa, Artificial Sequence
  Cordax peptide screen
AVVTGV

SEQ ID NO: 134 - protein, 6 aa, Artificial Sequence
  Cordax peptide screen
GAIIGL

SEQ ID NO: 135 - protein, 6 aa, Artificial Sequence
  Cordax peptide screen
VHGVTT

SEQ ID NO: 136 - protein, 6 aa, Artificial Sequence
  Cordax peptide screen
GNNQGS

SEQ ID NO: 137 - protein, 6 aa, Artificial Sequence
  Cordax peptide screen
NNQGSN

SEQ ID NO: 138 - protein, 6 aa, Artificial Sequence
  Cordax peptide screen
GYLLGS

SEQ ID NO: 139 - protein, 6 aa, Artificial Sequence
  Cordax peptide screen
IFQINS

SEQ ID NO: 140 - protein, 6 aa, Artificial Sequence
  Cordax peptide screen
VTGVTA

SEQ ID NO: 141 - protein, 6 aa, Artificial Sequence
  Cordax peptide screen
AALQSS

SEQ ID NO: 142 - protein, 6 aa, Artificial Sequence
  Cordax peptide screen
HGVTTV

SEQ ID NO: 143 - protein, 6 aa, Artificial Sequence
  Cordax peptide screen
KGAIIG

SEQ ID NO: 144 - protein, 6 aa, Artificial Sequence
  Cordax peptide screen
SSTSAA

SEQ ID NO: 145 - protein, 6 aa, Artificial Sequence
  Cordax peptide screen
NVGSNT

SEQ ID NO: 146 - protein, 6 aa, Artificial Sequence
  Cordax peptide screen
TGVTAV

SEQ ID NO: 147 - protein, 6 aa, Artificial Sequence
  Cordax peptide screen
NFGTFS

SEQ ID NO: 148 - protein, 6 aa, Artificial Sequence
  Cordax peptide screen
LTIITL

SEQ ID NO: 149 - protein, 6 aa, Artificial Sequence
  Cordax peptide screen
IAALLS

SEQ ID NO: 150 - protein, 6 aa, Artificial Sequence
  Cordax peptide screen
GDVIEV

SEQ ID NO: 151 - protein, 6 aa, Artificial Sequence
  Cordax peptide screen
VQIINK

SEQ ID NO: 152 - protein, 6 aa, Artificial Sequence
  Cordax peptide screen
VGGVVI

SEQ ID NO: 153 - protein, 6 aa, Artificial Sequence
  Cordax peptide screen
AMMAAA

SEQ ID NO: 154 - protein, 6 aa, Artificial Sequence
  Cordax peptide screen
FGAILS

SEQ ID NO: 155 - protein, 6 aa, Artificial Sequence
  Cordax peptide screen
VVTGVT

SEQ ID NO: 156 - protein, 6 aa, Artificial Sequence
  Cordax peptide screen
GVTTVA

SEQ ID NO: 157 - protein, 6 aa, Artificial Sequence
  Cordax peptide screen
GNNSYS

SEQ ID NO: 158 - protein, 6 aa, Artificial Sequence
  Cordax peptide screen
AIIGLM

SEQ ID NO: 159 - protein, 6 aa, Artificial Sequence
  Cordax peptide screen
DSVISL

SEQ ID NO: 160 - protein, 6 aa, Artificial Sequence
  Cordax peptide screen
SVISLS

SEQ ID NO: 161 - protein, 6 aa, Artificial Sequence
  Cordax peptide screen
SVISLS

SEQ ID NO: 162 - protein, 6 aa, Artificial Sequence
  Cordax peptide screen
TIITLE

SEQ ID NO: 163 - protein, 6 aa, Artificial Sequence
  Cordax peptide screen
TAVVTN

SEQ ID NO: 164 - protein, 6 aa, Artificial Sequence
  Cordax peptide screen
KGISVH

SEQ ID NO: 165 - protein, 6 aa, Artificial Sequence
  Cordax peptide screen
TIAALL

SEQ ID NO: 166 - protein, 6 aa, Artificial Sequence
  Cordax peptide screen
AALLSP

SEQ ID NO: 167 - protein, 6 aa, Artificial Sequence
  Cordax peptide screen
GVVTSE

SEQ ID NO: 168 - protein, 6 aa, Artificial Sequence
  Cordax peptide screen
GFNGGF

SEQ ID NO: 169 - protein, 6 aa, Artificial Sequence
  Cordax peptide screen
NFGEFS

SEQ ID NO: 170 - protein, 6 aa, Artificial Sequence
  Cordax peptide screen
MMHFGN

SEQ ID NO: 171 - protein, 6 aa, Artificial Sequence
  Cordax peptide screen
LVHSSN

SEQ ID NO: 172 - protein, 6 aa, Artificial Sequence
  APR
LATVYV

SEQ ID NO: 173 - protein, 6 aa, Artificial Sequence
  Capping peptide
LATKYV

SEQ ID NO: 174 - protein, 6 aa, Artificial Sequence
  Capping peptide
LRTVYV

SEQ ID NO: 175 - protein, 6 aa, Artificial Sequence
  Capping peptide
LWTVYV

SEQ ID NO: 176 - protein, 6 aa, Artificial Sequence
  Capping peptide
LYTVYV

SEQ ID NO: 177 - protein, 6 aa, Artificial Sequence
  Capping peptide
LFTVYV

SEQ ID NO: 178 - protein, 6 aa, Artificial Sequence
  Aggregation inducer
LLTVYV

SEQ ID NO: 179 - protein, 6 aa, Artificial Sequence
  Aggregation inducer
LALVYV

SEQ ID NO: 180 - protein, 6 aa, Artificial Sequence
  Aggregation inducer
LATLYV

SEQ ID NO: 181 - protein, 6 aa, Artificial Sequence
  Aggregation inducer
LATVYM

SEQ ID NO: 182 - protein, 6 aa, Artificial Sequence
  Aggregation inducer
LAIVYV